(19) United States
(12) Patent Application Publication
(10) Pub. No.: US 2009/0149458 A1
(43) Pub. Date: Jun. 11, 2009

(54) PYRROLO[3,2-d]PYRIMIDINE COMPOUNDS AND THEIR USE AS PI3 KINASE AND mTOR KINASE INHIBITORS

(75) Inventors: Zecheng CHEN, New City, NY (US); Aranapakam Mudumbai VENKATESAN, Rego Park, NY (US); Semiramis Ayral-Kaloustian, Tarrytown, NY (US); Tarek Subayl Mansour, New City, NY (US); Christoph Martin Dehnhardt, New York, NY (US); Arie Zask, New York, NY (US); Jeroen Cunera Verheijen, Highland Mills, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/276,459

(22) Filed: Nov. 24, 2008

Related U.S. Application Data
(60) Provisional application No. 60/990,424, filed on Nov. 27, 2007.

(51) Int. Cl. A61K 31/5377; C07D 487/04; A61P 35/00

(52) U.S. Cl. 514/234.2; 544/117

(57) ABSTRACT

A pyrrolo[3,2-d]pyrimidine compound, such as a compound of the formula (I):

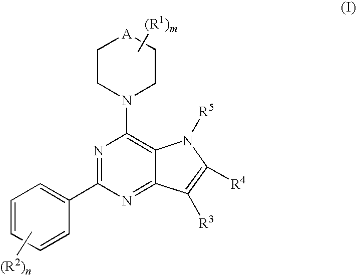

or a pharmaceutically acceptable salt thereof, wherein the constituent variables are as defined herein, compositions comprising the compounds, and methods for making and using the compounds.

### FIELD OF THE INVENTION

The invention relates to pyrrolo[3,2-d]pyrimidine compounds, compositions comprising a pyrrolo[3,2-d]pyrimidine compound, and methods for treating PI3K-related diseases comprising the administration of an effective amount of a pyrrolo[3,2-d]pyrimidine compound. The invention also relates to methods for treating mTOR-related diseases comprising the administration of an effective amount of a pyrrolo[3,2-d]pyrimidine compound.

### BACKGROUND OF THE INVENTION

Throughout this application, various publications are referenced. The disclosures of these publications in their entireties are hereby incorporated by reference into this application in order to more fully describe the state of the art as known to those skilled therein as of the date of the invention described and claimed herein.

Mammalian Target of Rapamycin, mTOR, is a cell-signaling protein that regulates the response of tumor cells to nutrients and growth factors, as well as controlling tumor blood supply through effects on Vascular Endothelial Growth Factor, VEGF. Inhibitors of mTOR starve cancer cells and shrink tumors by inhibiting the effect of mTOR. All mTOR inhibitors bind to the mTOR kinase. This has at least two important effects. First, mTOR is a downstream mediator of the PI3K/Akt pathway. The PI3K/Akt pathway is thought to be over activated in numerous cancers and may account for the widespread response from various cancers to mTOR inhibitors. The over-activation of the upstream pathway would normally cause mTOR kinase to be over activated as well. However, in the presence of mTOR inhibitors, this process is blocked. The blocking effect prevents mTOR from signaling to downstream pathways that control cell growth. Over-activation of the PI3K/Akt kinase pathway is frequently associated with mutations in the PTEN gene, which is common in many cancers and may help predict what tumors will respond to mTOR inhibitors. The second major effect of mTOR inhibition is anti-angiogenesis, via the lowering of VEGF levels.

In lab tests, certain chemotherapy agents were found to be more effective in the presence of mTOR inhibitors. George, J. N., et al., Cancer Research, 61, 1527-1532, 2001. Additional lab results have shown that some rhabdomyosarcoma cells die in the presence of mTOR inhibitors. The complete functions of the mTOR kinase and the effects of mTOR inhibition are not completely understood.

Phosphatidylinositol (hereinafter abbreviated as “PI”) is one of the phospholipids in cell membranes. In recent years it has become clear that PI plays an important role also in intracellular signal transduction. It is well recognized in the art that especially PI(4,5) bisphosphate (PI(4,5)P2) is degraded into diacylglycerol and inositol (1,4,5) triphosphate by phospholipase C to induce activation of protein kinase C and intracellular calcium mobilization, respectively [M. J. Berridge et al., Nature, 312, 315 (1984); Y. Nishizuka, Science, 225, 1365 (1984)].

In the late 1980s, phosphatidylinositol-3 kinase (“PI3K”) was found to be an enzyme that phosphorylates the 3-position of the inositol ring of phosphatidylinositol [D. Whitman et al., Nature, 332, 664 (1988)].

When PI3K was discovered, it was originally considered to be a single enzyme. Recently however, it was clarified that a plurality of subtypes are present in PI3K. Three major classes of PI3Ks have now been identified on the basis of their in vitro substrate specificity [B. Vanhaesebroeck, Trend in Biol. Sci., 22, 267 (1997)].

Substrates for class I PI3Ks are PI, PI(4)P and PI(4,5)P2. In these substrates, PI(4,5)P2 is the most advantageous substrate in cells. Class I PI3Ks are further divided into two groups, class Ia and class Ib, in terms of their activation mechanism. Class Ia PI3Ks, which include PI3K p110α, p110β, and p110δ subtypes, are activated in the tyrosine kinase system. Class Ib PI3K is a p110γ subtype activated by a G protein-coupled receptor.

PI and PI(4)P are known as substrates for class II PI3Ks but PI(4,5)P2 is not a substrate for the enzymes of this class. Class II PI3Ks include PI3K C2α, C2β and C2γ subtypes, which are characterized by containing C2 domains at the C terminus, implying that their activity will be regulated by calcium ions.

The substrate for class III PI3Ks is PI only. A mechanism for activation of the class III PI3Ks is not clarified yet. Because each subtype has its own mechanism for the regulating activity, it is considered that the respective subtypes will be activated depending on their respective stimuli specific to each of them.

In the PI3K subtypes, the class Ia subtype has been most extensively investigated to date. The three subtypes of class Ia are hetero dimers of a catalytic 1110-kDa subunit and regulatory subunits of 85 kDa and 55 kDa. The regulatory subunits contain SH2 domains and bind to tyrosine residues phosphorylated by growth factor receptors with a tyrosine kinase activity or oncogene products, thereby inducing the PI3K activity of the p110 catalytic subunit. Thus, the class Ia subtypes are considered to be associated with cell proliferation and carcinogenesis. Furthermore, the class Ia PI3K subtypes bind to activated ras oncogene to express their enzyme activity. It has been confirmed that the activated ras oncogene is present in many cancers, suggesting a role of class Ia PI3Ks in carcinogenesis.

There are three mTOR inhibitors, which have progressed into clinical trials. These compounds are Wyeth's Torisel, also known as 42-(3-hydroxy-2-(hydroxymethyl)-rapamycin 2-methylpropanoate, CCI-779 or Temsirolimus; Novartis' Everolimus, also known as 42-O-(2-hydroxyethyl)-rapamycin, or RAD 001; and Ariad's AP23573 also known as 42-(dimethylphopsinoyl)-rapamycin. The FDA has approved Torisel for the treatment of advanced renal cell carcinoma. In addition, Torisel is active in a NOS/SCID xenograft mouse model of acute lymphoblastic leukemia [Teachey et al, Blood, 107(3), 1149-1155, 2006]. Everolimus is in a phase II clinical study for patients with Stage 1V malignant melanoma. AP23573 has been given orphan drug and fast-track status by the FDA for treatment of soft-tissue and bone sarcomas.

The three mTOR inhibitors have non-linear, although reproducible pharmacokinetic profiles. Mean area under the curve (AUC) values for these drugs increase at a less than dose related way. The three compounds are all semi-synthetic derivatives of the natural macrolide antibiotic rapamycin. It would be desirable to find fully synthetic compounds, which inhibit mTOR that are more potent and exhibit improved pharmacokinetic behaviors.

As explained above, PI3K inhibitors and mTOR inhibitors are expected to be novel types of medicaments useful against cell proliferation disorders, especially as carcinostatic agents. Thus, it would be advantageous to have new PI3K inhibitors and mTOR inhibitors as potential treatment regimens for PI3K- and mTOR-related diseases. The instant invention is directed to these and other important ends.

### SUMMARY OF THE INVENTION

In one aspect, the invention provides compounds of the Formula (I):

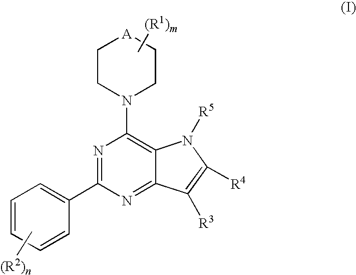

or a pharmaceutically acceptable salt thereof, wherein the constituent variables are as defined below.

In another aspect, the invention provides compounds of the Formula (VIII):

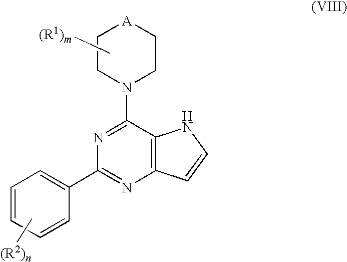

or a pharmaceutically acceptable salt thereof, wherein the constituent variables are as defined below.

In another aspect, the invention provides compounds of the Formula (XI):

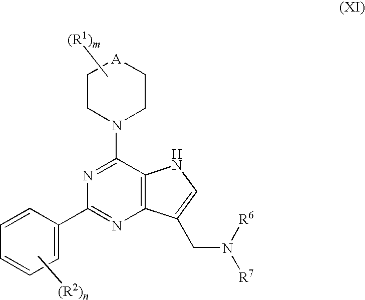

or a pharmaceutically acceptable salt thereof, wherein the constituent variables are as defined below.

In another aspect, the invention provides compounds of the Formula (XVI):

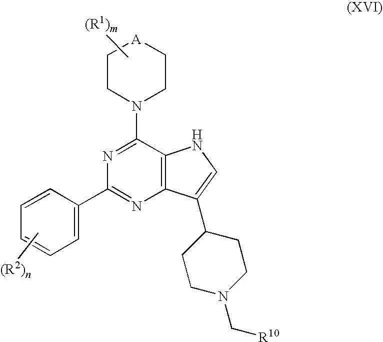

or a pharmaceutically acceptable salt thereof, wherein the constituent variables are as defined below.

In another aspect, the invention provides compounds of the Formula (XIX):

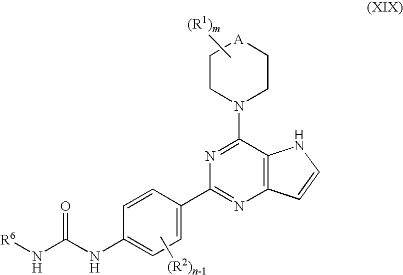

or a pharmaceutically acceptable salt thereof, wherein the constituent variables are as defined below.

In another aspect, the invention provides compounds of the Formula (XX):

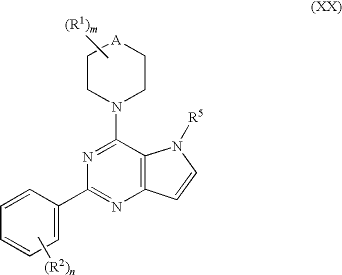

or a pharmaceutically acceptable salt thereof, wherein the constituent variables are as defined below.

In another aspect, the invention provides compounds of the Formula (XXIV):

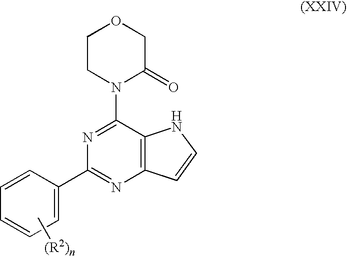

or a pharmaceutically acceptable salt thereof, wherein the constituent variables are as defined below.

In other aspects, the invention provides pharmaceutical compositions comprising compounds or pharmaceutically acceptable salts of compounds of the present invention and a pharmaceutically acceptable carrier.

In further aspects, the invention provides compounds or pharmaceutically acceptable salts of the compounds of the present invention that are useful as PI3K inhibitors, and methods for inhibiting PI3K using the compounds or pharmaceutically acceptable salts thereof.

In further aspects, the invention provides compounds or pharmaceutically acceptable salts of the compounds of the present invention that are useful as mTOR inhibitors, and methods for inhibiting mTOR using the compounds or pharmaceutically acceptable salts thereof.

In one embodiment, the invention provides methods for treating a PI3K-related disorder, comprising administering to a mammal in need thereof the compounds or pharmaceutically acceptable salts of compounds of the present invention in an amount effective to treat a PI3K-related disorder.

In one embodiment, the invention provides methods for treating an mTOR-related disorder, comprising administering to a mammal in need thereof, the compounds or pharmaceutically acceptable salts of compounds of the present invention in an amount effective to treat an mTOR-related disorder.

In other aspects, the invention provides further methods of synthesizing the compounds or pharmaceutically acceptable salts of compounds of the present invention.

### DETAILED DESCRIPTION OF THE INVENTION

In one aspect, the invention provides compounds of the Formula (I):

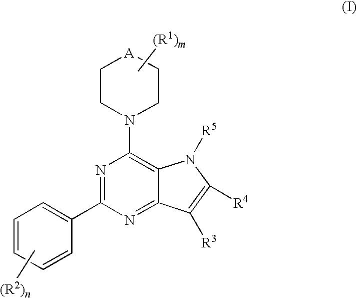

or a pharmaceutically acceptable salt thereof, wherein
R¹ is independently C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₂-C₆alkenyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O) (C₁-C₆alkyl), —NHC(O) (C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₂-C₆alkynyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₃-C₈cycloalkyl optionally substituted with from 1 to 3 substituents independently selected from C₁-C₅alkyl, halo, halo(C₁-C₆alkyl)-, hydroxyl, —O—C₁-C₅alkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, carboxyamidoalkyl- and —NO₂; C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; or C₁-C₉heteroaryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂;
or two R¹ groups on the same carbon atom, when taken together with the carbon to which they are attached, form a carbonyl (C═O) group or two R¹ groups on the same carbon atom can be replaced by an alkylenedioxy group so that the alkylenedioxy group, when taken together with the carbon atom to which it is attached, form a 5- to 7-membered heterocycle containing two oxygen atoms;

### A is —O—, —CH₂O—, —S—, —S(O)—, or S(O)₂—;

m is 0, 1, or 2;
R² is independently halogen; C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₁-C₆alkoxy optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₁-C₆alkoxycarbonyl; C₂-C₆alkenyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₂-C₆alkynyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O) (C₁-C₆alkyl), —NHC(O) (C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₃-C₈cycloalkyl optionally substituted with from 1 to 3 substituents independently selected from C₁-C₅alkyl, halo, halo(C₁-C₆alkyl)-, hydroxyl, —O—C₁-C₅alkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, carboxyamidoalkyl- and —NO₂; C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; C₁-C₉heteroaryl optionally substituted with from 1 to 3 substituents independently selected from C₁-C₅alkyl, halo, halo(C₁-C₆alkyl)-, hydroxyl, C₁-C₅hydroxylalkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; hydroxyl; NR⁶R⁷; NO₂; CN; CO₂H; CF₃; CF₃O; C₁-C₆alkylthio; —SO₂NR⁶R⁷; —C(O)NR⁶R⁷; —NHC(O)NR⁶R⁷; —NHC(O)OR⁸; —NH(SO₂)NH—C₁-C₆alkyl; —NH(SO₂)NH—C₆-C₁₄aryl; —NHC(S)—NH—C₁-C₆alkyl; —N═C(S—C₁-C₆alkyl)(NH—C₁-C₆alkyl); —S(O)ₚ—C₆-C₁₄aryl; —S(O)ₚ—C₁-C₉heteroaryl; or —N(H)—C(═N—(CN))—(O—C₆-C₁₄aryl);
n is 1,2,3,4, or 5;
each p is independently 1 or 2;
R⁶ and R⁷ are each independently H; C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, —NO₂, R¹¹R¹²NC(O)—, R¹¹R¹²NNHC(O)—, R¹¹O—, R¹¹R¹²N—, R¹¹R¹²NS(O)₂—, R¹¹S(O)₂NR¹²—, R¹¹R¹²NC(O)NH, R¹¹S—, R¹¹S(O)—, R¹¹S(O)₂—, and R¹¹C(O)—; C₁-C₉heteroaryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, —NO₂, R¹¹R¹²NC(O)—, R¹¹R¹²NNHC(O)—, R¹¹O—, R¹¹R¹²N—, R¹¹R¹²NS(O)₂—, R¹¹S(O)₂NR¹²—, R¹¹R¹²NC(O)NH, R¹¹S—, R¹¹S(O)—, R¹¹S(O)₂—, and R¹¹C(O)—; C₃-C₈cycloalkyl optionally substituted with from 1 to 3 substituents independently selected from C₁-C₅alkyl, halo, halo(C₁-C₆alkyl)-, hydroxyl, —O—C₁-C₅alkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, carboxyamidoalkyl- and —NO₂; or C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl;
or R⁶ and R⁷ when taken together with the nitrogen to which they are attached form a 3- to 7-membered nitrogen containing heterocycle wherein up to two of the carbon atoms of the heterocycle can be replaced with —N(R⁹)—, —O—, or —S(O)ₚ—;
R⁸ is C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; or C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂;
R⁹ is hydrogen; C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₃-C₈cycloalkyl optionally substituted with from 1 to 3 substituents independently selected from C₁-C₅alkyl, halo, halo(C₁-C₆alkyl)-, hydroxyl, —O—C₁-C₅alkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, carboxyamidoalkyl- and —NO₂; C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; C₁-C₉heteroaryl optionally substituted with from 1 to 3 substituents independently selected from C₁-C₅alkyl, halo, halo(C₁-C₆alkyl)-, hydroxyl, C₁-C₅hydroxylalkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; amino(C₁-C₆alkyl)-; or C₆-C₁₄arylamino;
R¹¹ and R¹² are each independently H, C₁-C₆alkoxy-, C₁-C₆alkyl-, C₁-C₆alkoxy(C₂-C₆alkylene)-, (C₁-C₆alkyl)amino-C₂-C₆alkylene-, di(C₁-C₆alkyl)amino-C₂-C₆alkylene-, C₂-C₆alkenyl, C₂-C₆alkynyl, C₆-C₁₄aryl-, (C₆-C₁₄aryl)alkyl-, C₃-C₈cycloalkyl-, C₁-C₉heteroaryl-, (C₁-C₉heteroaryl)alkyl-, C₁-C₉heterocyclyl- optionally substituted by C₁-C₆alkyl-, or heterocyclyl(C₁-C₆alkyl-);
or R¹¹ and R¹², when taken together with the nitrogen to which they are attached, form a 3- to 7-membered heterocycle wherein up to two of the carbon atoms of the heterocycle are optionally replaced with —N(H)—, —N(C₁-C₆alkyl)-, —N(C₃-C₈cycloalkyl)-, —N(C₆-C₁₄aryl)-, —N(C₁-C₉heteroaryl)-, —S—, —SO—, —S(O)₂—, or —O— and wherein any carbon atom of the heterocycle is optionally substituted with from 1 or 2 substituents independently selected from C₁-C₆alkyl-, H₂N—, (C₁-C₆alkyl)amino-, di(C₁-C₆alkyl)amino-, and C₁-C₉heterocyclyl-;
R³, R⁴, and R⁵ are independently H; C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₂-C₆alkenyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₇cycloalkyl; C₂-C₆alkynyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; C₁-C₉heteroaryl optionally substituted with from 1 to 3 substituents independently selected from C₁-C₅alkyl, halo, halo(C₁-C₆alkyl)-, hydroxyl, C₁-C₅hydroxylalkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; NR⁶R⁷; C₁-C₆alkoxycarbonyl; perfluoroalkyl; —S(O)ₚ—C₆-C₁₄aryl; —S(O)ₚ—C₁-C₆alkyl; C(O)NR⁶R⁷; optionally substituted (C₆-C₁₄)arylalkyl- optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; heterocyclyl(C₁-C₆alkyl)- with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, (C₆-C₁₄aryl)alkyl-, —C(O)OH, —C(O)OC₁-C₆alkyl, —C(O)C₁-C₆alkyl, C₆-C₁₄ aryl, C₁-C₉heteroaryl, and C₃-C₈ cycloalkyl, wherein one of the CH₂ groups in the alkyl chain of the heterocyclyl(C₁-C₆alkyl)- can optionally be replaced by a NH group; 4- to 7-membered monocyclic heterocycle group optionally substituted with from 1 to 3 substituents independently selected from C₁-C₈acyl, C₁-C₆alkyl, heterocyclyl(C₁-C₆alkyl)-, wherein the ring portion of the heterocyclyl(C₁-C₆alkyl)- group is optionally substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, (C₆-C₁₄aryl)alkyl, wherein the ring portion of the (C₆-C₁₄aryl)alkyl group is optionally substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O) (C₁-C₆alkyl), (C₆-C₁₄)arylalkyl-O—C(O)—, (C₁-C₆-alkoxycarbonyl)-NH—(C₁-C₆)alkylene-, N-alkylamido-, —C(O)NH₂, (C₁-C₆alkyl)N-alkylamido-, and —NO₂; or C₃-C₈cycloalkyl optionally substituted with from 1 to 3 substituents independently selected from C₁-C₅alkyl, halo, halo(C₁-C₆alkyl)-, hydroxyl, —O—C₁-C₅alkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, carboxyamidoalkyl- and —NO₂.

In another aspect, the invention provides compounds of the Formula (I) wherein R⁶ and R⁷ are each independently H; C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; C₁-C₉heteroaryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; C₃-C₈cycloalkyl optionally substituted with from 1 to 3 substituents independently selected from C₁-C₅alkyl, halo, halo(C₁-C₆alkyl)-, hydroxyl, —O—C₁-C₅alkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, carboxyamidoalkyl- and —NO₂; or C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

In another aspect, the invention provides compounds of the Formula (VIII):

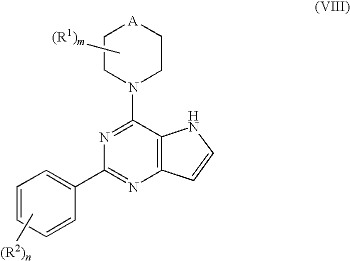

or a pharmaceutically acceptable salt thereof, wherein A, R¹, R², m and n are as defined above for Formula (I).

In another aspect, the invention provides compounds of the Formula (XI):

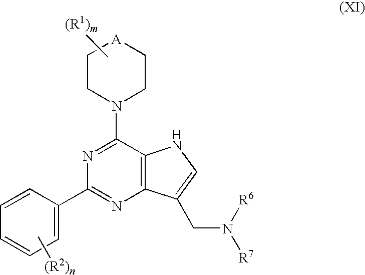

or a pharmaceutically acceptable salt thereof, wherein A, R¹, R², R⁶, R⁷, m, and n are as defined above for Formula (I).

In another aspect, the invention provides compounds of the Formula (XVI):

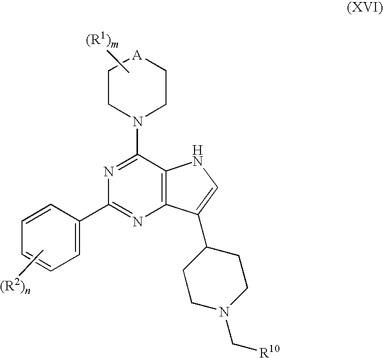

or a pharmaceutically acceptable salt thereof, wherein A, R¹, R², m, and n are as defined above for Formula I, and R¹⁰ is C₁-C₆alkyl or C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂.

In another aspect, the invention provides compounds of the Formula (XIX):

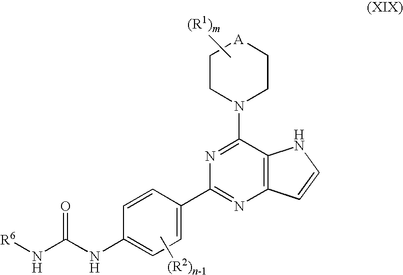

or a pharmaceutically acceptable salt thereof, wherein A, R¹, R², R⁶, m, and n are as defined above for Formula I.

In another aspect, the invention provides compounds of the Formula (XX):

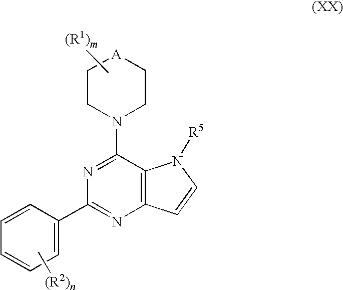

or a pharmaceutically acceptable salt thereof, wherein A, R¹, R², R⁵, m, and n are as defined above for Formula I.

In another aspect, the invention provides compounds of the Formula (XXIV):

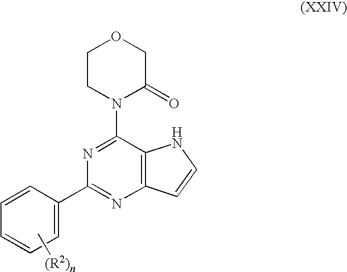

or a pharmaceutically acceptable salt thereof, wherein R², and n are as defined above for Formula I.

In one embodiment, m is 0.

In one embodiment, n is 1.

In one embodiment, A is —O—.

In one embodiment, R² is an optionally substituted urea of the formula —NHC(O)NR⁶R⁷, wherein R⁶ and R⁷ are each independently H; C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; C₁-C₉heteroaryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; C₃-C₈cycloalkyl optionally substituted with from 1 to 3 substituents independently selected from C₁-C₅alkyl, halo, halo(C₁-C₆alkyl)-, hydroxyl, —O—C₁-C₅alkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, carboxyamidoalkyl- and —NO₂; or C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl;

or R⁶ and R⁷ when taken together with the nitrogen to which they are attached form a 3- to 7-membered nitrogen containing heterocycle wherein up to two of the carbon atoms of the heterocycle can be replaced with —N(R⁹)—, —O—, or —S(O)ₚ—;
R⁹ is hydrogen; C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₃-C₈cycloalkyl optionally substituted with from 1 to 3 substituents independently selected from C₁-C₅alkyl, halo, halo(C₁-C₆alkyl)-, hydroxyl, —O—C₁-C₅alkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, carboxyamidoalkyl- and —NO₂; C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; C₁-C₉heteroaryl optionally substituted with from 1 to 3 substituents independently selected from C₁-C₅alkyl, halo, halo(C₁-C₆alkyl)-, hydroxyl, C₁-C₅hydroxylalkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; amino(C₁-C₆alkyl)-; or C₆-C₁₄arylamino.

In another embodiment, R² is C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

In another embodiment, R² is the optionally substituted C₁-C₆alkyl group —CH₂OH.

In another embodiment, R² is OH.

In another embodiment, R² is OH in the meta position.

In another embodiment, R² is amino.

In one embodiment, R³ is H.

In another embodiment, R³ is amino(C₁-C₆alkyl)optionally substituted with from 1 to 3 substituents independently selected from C₁-C₆alkoxy, C₆-C₁₄aryl, C₁-C₉heteroaryl, C₃-C₈cycloalkyl, and C₁-C₆alkyl.

In another embodiment, R³ is di(C₁-C₆alkyl)aminomethyl.

In another embodiment, R³ is dimethylaminomethyl.

In another embodiment, R⁴ is H.

In another embodiment, R⁵ is H.

Illustrative compounds of Formula (I) are exemplified by the following compounds:
2-[3-(benzyloxy)phenyl]-4-morpholin-4-yl-5H-pyrrolo[3,2-d]pyrimidine;
3-(4-morpholin-4-yl-5H-pyrrolo[3,2-d]pyrimidin-2-yl)phenol;
3-(4-morpholin-4-yl-5H-pyrrolo[3,2-d]pyrimidin-2-yl)phenyl]methanol;
2-(3-methylphenyl)-4-morpholin-4-yl-5H-pyrrolo[3,2-d]pyrimidine;
3-(4-morpholin-4-yl-5H-pyrrolo[3,2-d]pyrimidin-2-yl)aniline;
1-methyl-3-[4-(4-morpholin-4-yl-5H-pyrrolo[3,2-d]pyrimidin-2-yl)phenyl]urea;
{3-[4-morpholin-4-yl-7-(pyrrolidin-1-ylmethyl)-5H-pyrrolo[3,2-d]pyrimidin-2-yl]phenyl}methanol;
[3-(4-morpholin-4-yl-7-{[(2-piperidin-1-ylethyl)amino]methyl}-5H-pyrrolo[3,2-d]pyrimidin-2-yl)phenyl]methanol;
[3-(4-morpholin-4-yl-7-{[(pyridin-3-ylmethyl)amino]methyl}-5H-pyrrolo[3,2-d]pyrimidin-2-yl)phenyl]methanol;
3-{7-[(4-methylpiperazin-1-yl)methyl]-4-morpholin-4-yl-5H-pyrrolo[3,2-d]pyrimidin-2-yl}phenyl)methanol;
{3-[4-morpholin-4-yl-7-(piperazin-1-ylmethyl)-5H-pyrrolo[3,2-d]pyrimidin-2-yl]phenyl}methanol;
3-{7-[(dimethylamino)methyl]-4-morpholin-4-yl-5H-pyrrolo[3,2-d]pyrimidin-2-yl}phenyl)methanol;
3-{7-[(diethylamino)methyl]-4-morpholin-4-yl-5H-pyrrolo[3,2-d]pyrimidin-2-yl}phenyl)methanol;
3-{4-morpholin-4-yl-7-[(4-pyrimidin-2-ylpiperazin-1-yl)methyl]-5H-pyrrolo[3,2-d]pyrimidin-2-yl}phenyl)methanol;
3-{7-[(4-benzylpiperazin-1-yl)methyl]-4-morpholin-4-yl-5H-pyrrolo[3,2-d]pyrimidin-2-yl}phenyl)methanol;
3-(4-morpholin-4-yl-7-{[(pyridin-3-ylmethyl)amino]methyl}-5H-pyrrolo[3,2-d]pyrimidin-2-yl)phenol;
3-[4-morpholin-4-yl-7-(pyrrolidin-1-ylmethyl)-5H-pyrrolo[3,2-d]pyrimidin-2-yl]phenol;
3-{7-[(dimethylamino)methyl]-4-morpholin-4-yl-5H-pyrrolo[3,2-d]pyrimidin-2-yl}phenol;
3-{7-[(diethylamino)methyl]-4-morpholin-4-yl-5H-pyrrolo[3,2-d]pyrimidin-2-yl}phenol;
3-{4-morpholin-4-yl-7-[(4-pyrimidin-2-ylpiperazin-1-yl)methyl]-5H-pyrrolo[3,2-d]pyrimidin-2-yl}phenol;
2-[3-(benzyloxy)phenyl]-4-morpholin-4-yl-7-(1,2,3,6-tetrahydropyridin-4-yl)-5H-pyrrolo[3,2-d]pyrimidine;
3-(4-morpholin-4-yl-7-piperidin-4-yl-5H-pyrrolo[3,2-d]pyrimidin-2-yl)phenol;
3-{7-[1-(4-fluorobenzyl)piperidin-4-yl]-4-morpholin-4-yl-5H-pyrrolo[3,2-d]pyrimidin-2-yl}phenol;
{3-[4-morpholin-4-yl-7-(1,2,3,6-tetrahydropyridin-4-yl)-5H-pyrrolo[3,2-d]pyrimidin-2-yl]phenyl}methanol;
3-[7-(1-benzyl-1,2,3,6-tetrahydropyridin-4-yl)-4-morpholin-4-yl-5H-pyrrolo[3,2-d]pyrimidin-2-yl]phenyl}methanol;
3-[7-(1-benzylpiperidin-4-yl)-4-morpholin-4-yl-5H-pyrrolo[3,2-d]pyrimidin-2-yl]phenol;
3-{7-[1-(2-furylmethyl)piperidin-4-yl]-4-morpholin-4-yl-5H-pyrrolo[3,2-d]pyrimidin-2-yl}phenol;
3-{7-[1-(1H-imidazol-2-ylmethyl)piperidin-4-yl]-4-morpholin-4-yl-5H-pyrrolo[3,2-d]pyrimidin-2-yl}phenol;
3-[7-(1-isobutylpiperidin-4-yl)-4-morpholin-4-yl-5H-pyrrolo[3,2-d]pyrimidin-2-yl]phenol;
3-[7-(1-methylpiperidin-4-yl)-4-morpholin-4-yl-5H-pyrrolo[3,2-d]pyrimidin-2-yl]phenol;
3-[7-(1-cyclohexylpiperidin-4-yl)-4-morpholin-4-yl-5H-pyrrolo[3,2-d]pyrimidin-2-yl]phenol;
3-{7-[1-(2-fluorobenzyl)piperidin-4-yl]-4-morpholin-4-yl-5H-pyrrolo[3,2-d]pyrimidin-2-yl}phenol;
3-{4-morpholin-4-yl-7-[1-(1H-pyrrol-2-ylmethyl)piperidin-4-yl]-5H-pyrrolo[3,2-d]pyrimidin-2-yl}phenol;
3-{7-[1-(2-chloro-4-fluorobenzyl)piperidin-4-yl]-4-morpholin-4-yl-5H-pyrrolo[3,2-d]pyrimidin-2-yl}phenol;
3-(7-{1-[(6-chloropyridin-3-yl)methyl]piperidin-4-yl}-4-morpholin-4-yl-5H-pyrrolo[3,2-d]pyrimidin-2-yl)phenol;
tert-Butyl (2-{4-[2-(3-hydroxyphenyl)-4-morpholin-4-yl-5H-pyrrolo[3,2-d]pyrimidin-7-yl]piperidin-1-yl}ethyl)carbamate;
3-(4-morpholin-4-yl-7-{1-[(4-morpholin-4-ylpyridin-3-yl)methyl]piperidin-4-yl}-5H-pyrrolo[3,2-d]pyrimidin-2-yl)phenol;
3-{4-morpholin-4-yl-7-[1-(pyridin-2-ylmethyl)piperidin-4-yl]-5H-pyrrolo[3,2-d]pyrimidin-2-yl}phenol;
3-(7-{1-[(6-fluoropyridin-3-yl)methyl]piperidin-4-yl}-4-morpholin-4-yl-5H-pyrrolo[3,2-d]pyrimidin-2-yl)phenol;
1-[2-(dimethylamino)ethyl]-3-[3-(4-morpholin-4-yl-5H-pyrrolo[3,2-d]pyrimidin-2-yl)phenyl]urea;
1-(3-hydroxypropyl)-3-[3-(4-morpholin-4-yl-5H-pyrrolo[3,2-d]pyrimidin-2-yl)phenyl]urea;
1-[3-(1H-imidazol-1-yl)propyl]-3-[3-(4-morpholin-4-yl-5H-pyrrolo[3,2-d]pyrimidin-2-yl)phenyl]urea;
1-(2-furylmethyl)-3-[3-(4-morpholin-4-yl-5H-pyrrolo[3,2-d]pyrimidin-2-yl)phenyl]urea;
1-[3-(4-morpholin-4-yl-5H-pyrrolo[3,2-d]pyrimidin-2-yl)phenyl]-3-(pyridin-3-ylmethyl)urea;
[3-(5-methyl-4-morpholin-4-yl-5H-pyrrolo[3,2-d]pyrimidin-2-yl)phenyl]methanol;
methyl {2-[3-(hydroxymethyl)phenyl]-4-morpholin-4-yl-5H-pyrrolo[3,2-d]pyrimidin-5-yl}acetate;
{3-[5-methyl-4-morpholin-4-yl-7-(pyrrolidin-1-ylmethyl)-5H-pyrrolo[3,2-d]pyrimidin-2-yl]phenyl}methanol;
4-[2-(3-hydroxyphenyl)-5H-pyrrolo[3,2-d]pyrimidin-4-yl]morpholin-3-one.

Illustrative compounds of Formula (I) are exemplified by the following compounds:
1-[4-(4-morpholin-4-yl-5H-pyrrolo[3,2-d]pyrimidin-2-yl)phenyl]-3-pyridin-4-ylurea;
1-[4-(5-benzyl-4-morpholin-4-yl-5H-pyrrolo[3,2-d]pyrimidin-2-yl)phenyl]-3-pyridin-4-ylurea;
1-[4-(5-methyl-4-morpholin-4-yl-5H-pyrrolo[3,2-d]pyrimidin-2-yl)phenyl]-3-pyridin-4-ylurea;
1-[4-(4-morpholin-4-yl-5H-pyrrolo[3,2-d]pyrimidin-2-yl)phenyl]-3-{4-[(4-methylpiperazin-1-yl)carbonyl]phenyl}urea;
1-[4-(4-morpholin-4-yl-5H-pyrrolo[3,2-d]pyrimidin-2-yl)phenyl]-3-{4-[(4-ethylpiperazin-1-yl)carbonyl]phenyl}urea;
1-[4-(4-morpholin-4-yl-5H-pyrrolo[3,2-d]pyrimidin-2-yl)phenyl]-3-{4-[(4-isopropylpiperazin-1-yl)carbonyl]phenyl}urea;
1-[4-(4-morpholin-4-yl-5H-pyrrolo[3,2-d]pyrimidin-2-yl)phenyl]-3-{4-[(piperazin-1-yl)carbonyl]phenyl}urea;
1-[4-(4-morpholin-4-yl-5H-pyrrolo[3,2-d]pyrimidin-2-yl)phenyl]-3-{4-[(4-(dimethylamino)piperidin-1-yl)carbonyl]phenyl}urea;
N-[2-(dimethylamino)ethyl]-4-({[4-(4-morpholin-4-yl-5H-pyrrolo[3,2-d]pyrimidin-2-yl)phenyl]carbamoyl}amino)-N-methylbenzamide;
N-[2-(dimethylamino)ethyl]-4-({[4-(4-morpholin-4-yl-5H-pyrrolo[3,2-d]pyrimidin-2-yl)phenyl]carbamoyl}amino)benzamide-4-({[4-(4-morpholin-4-yl-5H-pyrrolo[3,2-d]pyrimidin-2-yl)phenyl]carbamoyl}amino)-N-(2-pyrrolidin-1-ylethyl)benzamide;
1-[4-(4-morpholin-4-yl-5H-pyrrolo[3,2-d]pyrimidin-2-yl)phenyl]-3-{4-[(4-pyrrolidin-1-ylpiperidin-1-yl)carbonyl]phenyl}ureal -[4-(5-methyl-4-morpholin-4-yl-5H-pyrrolo[3,2-d]pyrimidin-2-yl)phenyl]-3-{4-[(4-methylpiperazin-1-yl)carbonyl]phenyl}urea;
1-[4-(5-methyl-4-morpholin-4-yl-5H-pyrrolo[3,2-d]pyrimidin-2-yl)phenyl]-3-{4-[(4-ethylpiperazin-1-yl)carbonyl]phenyl}urea;
1-[4-(5-methyl-4-morpholin-4-yl-5H-pyrrolo[3,2-d]pyrimidin-2-yl)phenyl]-3-{4-[(4-isopropylpiperazin-1-yl)carbonyl]phenyl}urea;
1-[4-(5-methyl-4-morpholin-4-yl-5H-pyrrolo[3,2-d]pyrimidin-2-yl)phenyl]-3-{4-[(piperazin-1-yl)carbonyl]phenyl}urea;
1-[4-(5-methyl-4-morpholin-4-yl-5H-pyrrolo[3,2-d]pyrimidin-2-yl)phenyl]-3-{4-[(4-(dimethylamino)piperidin-1-yl)carbonyl]phenyl}urea;
N-[2-(dimethylamino)ethyl]-4-({[4-(5-methyl-4-morpholin-4-yl-5H-pyrrolo[3,2-d]pyrimidin-2-yl)phenyl]carbamoyl}amino)-N-methylbenzamide;
N-[2-(dimethylamino)ethyl]-4-({[4-(5-methyl-4-morpholin-4-yl-5H-pyrrolo[3,2-d]pyrimidin-2-yl)phenyl]carbamoyl}amino)benzamide-4-({[4-(5-methyl-4-morpholin-4-yl-5H-pyrrolo[3,2-d]pyrimidin-2-yl)phenyl]carbamoyl}amino)-N-(2-pyrrolidin-1-ylethyl)benzamide;
1-[4-(5-methyl-4-morpholin-4-yl-5H-pyrrolo[3,2-d]pyrimidin-2-yl)phenyl]-3-{4-[(4-pyrrolidin-1-ylpiperidin-1-yl)carbonyl]phenyl}urea;
1-[4-(5-ethyl-4-morpholin-4-yl-5H-pyrrolo[3,2-d]pyrimidin-2-yl)phenyl]-3-{4-[(4-methylpiperazin-1-yl)carbonyl]phenyl}urea;
1-[4-(5-ethyl-4-morpholin-4-yl-5H-pyrrolo[3,2-d]pyrimidin-2-yl)phenyl]-3-{4-[(4-ethylpiperazin-1-yl)carbonyl]phenyl}urea;
1-[4-(5-ethyl-4-morpholin-4-yl-5H-pyrrolo[3,2-d]pyrimidin-2-yl)phenyl]-3-{4-[(4-isopropylpiperazin-1-yl)carbonyl]phenyl}urea;
1-[4-(5-ethyl-4-morpholin-4-yl-5H-pyrrolo[3,2-d]pyrimidin-2-yl)phenyl]-3-{4-[(piperazin-1-yl)carbonyl]phenyl}urea;
1-[4-(5-ethyl-4-morpholin-4-yl-5H-pyrrolo[3,2-d]pyrimidin-2-yl)phenyl]-3-{4-[(4-(dimethylamino)piperidin-1-yl)carbonyl]phenyl}urea;
N-[2-(dimethylamino)ethyl]-4-({[4-(5-ethyl-4-morpholin-4-yl-5H-pyrrolo[3,2-d]pyrimidin-2-yl)phenyl]carbamoyl}amino)-N-methylbenzamide;
N-[2-(dimethylamino)ethyl]-4-({[4-(5-ethyl-4-morpholin-4-yl-5H-pyrrolo[3,2-d]pyrimidin-2-yl)phenyl]carbamoyl}amino)benzamide-4-({[4-(5-ethyl-4-morpholin-4-yl-5H-pyrrolo[3,2-d]pyrimidin-2-yl)phenyl]carbamoyl}amino)-N-(2-pyrrolidin-1-ylethyl)benzamide; and
1-[4-(5-ethyl-4-morpholin-4-yl-5H-pyrrolo[3,2-d]pyrimidin-2-yl)phenyl]-3-{4-[(4-pyrrolidin-1-ylpiperidin-1-yl)carbonyl]phenyl}urea.

The invention also includes pharmaceutical compositions comprising an effective amount of a pyrrolopyrimidine compound and a pharmaceutically acceptable carrier. The invention includes a pyrrolopyrimidine compound when provided as a pharmaceutically acceptable prodrug, hydrated salt, such as a pharmaceutically acceptable salt, or mixtures thereof.

In other aspects, the invention provides that the pharmaceutically acceptable carrier suitable for oral administration and the composition comprises an oral dosage form.

In other aspects, the invention provides a composition comprising a compound of Formula I; a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, lavendustin A, hydroxyzine, glatiramer acetate, interferon beta-1a, interferon beta-1b, and natalizumab and lavendustin A; and a pharmaceutically acceptable carrier.

In other aspects, the second compound is Avastin.

In other aspects, the invention provides a method of treating a PI3K-related disorder, comprising administering to a mammal in need thereof a compound of Formula I in an amount effective to treat a PI3K-related disorder.

In other aspects, the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the PI3K-related disorder is cancer.

In other aspects, the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

In other aspects, the invention provides a method of treating an mTOR-related disorder, comprising administering to a mammal in need thereof a compound of Formula I in an amount effective to treat an mTOR-related disorder.

In other aspects, the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the mTOR-related disorder is cancer.

In other aspects, the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

In other aspects, the invention provides a method of treating advanced renal cell carcinoma, comprising administering to a mammal in need thereof a compound of Formula I in an amount effective to treat advanced renal cell carcinoma.

In other aspects, the invention provides a method of treating acute lymphoblastic leukemia, comprising administering to a mammal in need thereof a compound of Formula I in an amount effective to treat acute lymphoblastic leukemia.

In other aspects, the invention provides a method of treating acute malignant melanoma, comprising administering to a mammal in need thereof a compound of Formula I in an amount effective to treat malignant melanoma.

In other aspects, the invention provides a method of treating soft-tissue or bone sarcoma, comprising administering to a mammal in need thereof a compound of Formula I in an amount effective to treat soft-tissue or bone sarcoma.

In other aspects, the invention provides a method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof a composition comprising a compound of Formula I; a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, and lavendustin A; and a pharmaceutically acceptable carrier. in an amount effective to treat the cancer.

In other aspects, the invention provides a method of inhibiting mTOR in a subject, comprising administering to a subject in need thereof a compound of Formula I in an amount effective to inhibit mTOR.

In other aspects, the invention provides a method of inhibiting PI3K in a subject, comprising administering to a subject in need thereof a compound of Formula I in an amount effective to inhibit PI3K.

In another aspect, the invention provides methods of synthesizing compounds of the formula (VIII) comprising:

a) reacting 6-methyl-5-nitro-2,4-dichloropyrimidine of the Formula (II):

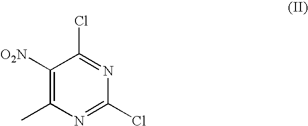

with a morpholine compound of the Formula (III);

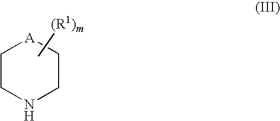

wherein R¹, A, and m are as defined in Formula (I) to give the chloropyrimidine intermediate of Formula (IV):

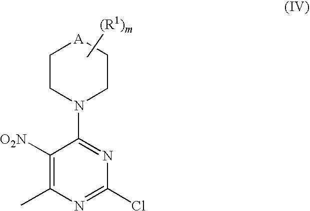

b) reacting the compound of Formula (IV) with a boronic acid of the structure (V):

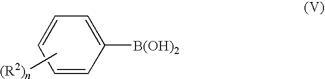

wherein R² and n are as defined in Formula (I) thereby providing a compound of the Formula (VI):

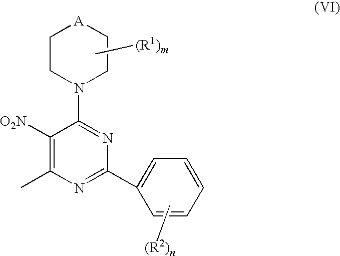

(c) reacting the compound of Formula (VI) with 1,1-dimethoxy-N,N-dimethylmethylamine (DMF-DMA) followed by reductive cyclization thereby providing a compound of the Formula (VIII):

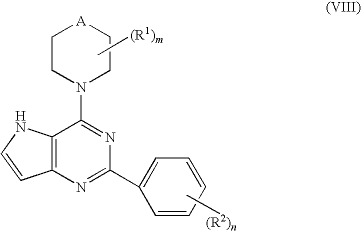

or a pharmaceutically acceptable salt thereof.

In another aspect, the invention provides methods of synthesizing compounds of the formula (I) comprising:

a) reacting 6-substituted-5-nitro-2,4-dihalopyrimidine of the Formula (XXV):

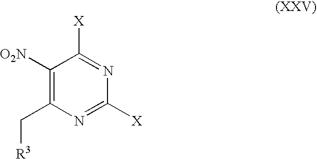

wherein R³ is as defined in Formula (I) and X is a leaving group with a morpholine compound of the Formula (III);

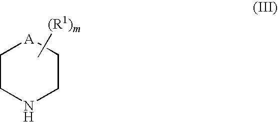

wherein R¹ and m are as defined in Formula (I) to give the halopyrimidine intermediate of Formula (XXVI):

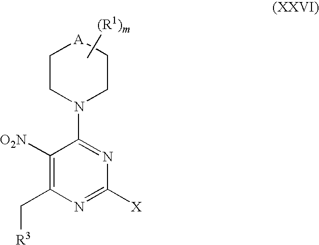

thereby providing a compound having the Formula (XXVI):
b) reacting the compound of Formula (XXVI) with a boronic acid of the structure (V):

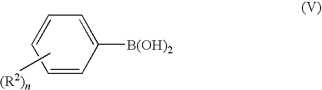

wherein R² and n are as defined in Formula (I) thereby providing a compound of the Formula (XXVII):

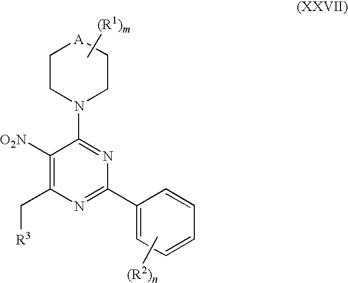

(c) reacting the compound of Formula (XXVII) with a 1,1-dimethoxy-N,N-dimethylalkylamine of the formula R⁴C(OCH₃)₂N(CH₃), followed by reductive cyclization thereby providing a compound of the Formula (XXIX):

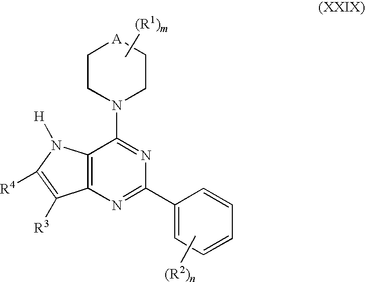

wherein R⁴ is as defined in Formula (I);
(d) reacting the compound of Formula (XXIX) at the pyrrole nitrogen by treating with sodium hydride and an alkylating agent R⁵X thereby providing a compound of the Formula (I):

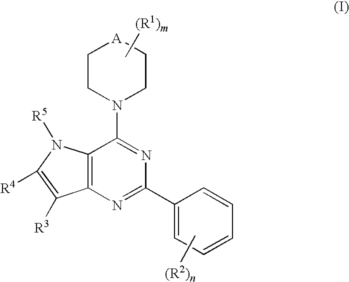

wherein X is a leaving group and R⁵ is as defined in Formula (I).

### DEFINITIONS

The following definitions are used in connection with the pyrrolo[3,2-d]pyrimidine compounds of the present invention:

“Acyl” refers to groups of carbon atoms in a straight, branched, or cyclic configuration or a combination thereof, attached to the parent structure through a carbonyl functionality e.g. of 1-10 carbon atoms, 1-8 carbon atoms, 1-6 carbon atoms, or 1-4 carbon atoms. The number of carbon atoms in the group does not include the carbon atom included in the linking carbonyl functionality. Such groups may be saturated or unsaturated, aliphatic or aromatic, and carbocyclic or heterocyclic. Examples of C₁-C₈acyl include acetyl-, benzoyl-, nicotinoyl, propionyl-, isobutyryl-, and oxalyl-. An acyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl.

“Alkenyl” refers to a straight or branched chain unsaturated hydrocarbon containing and at least one double bond e.g. of 2-10 carbon atoms, 2-6 carbon atoms, or 2-4 carbon atoms. Examples of a C₂-C₁₀alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, isohexene, 1-heptene, 2-heptene, 3-heptene, 1-octene, 2-octene, 3-octene, 4-octene, 1-nonene, 2-nonene, 3-nonene, 4-nonene, 1-decene, 2-decene, 3-decene, 4-decene and 5-decene. An alkenyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

“Alkoxy” refers to the group R—O— where R is an alkyl group, as defined below, e.g. of 1-10 carbon atoms, 1-6 carbon atoms, or 1-4 carbon atoms. Exemplary C₁-C₆alkoxy groups include but are not limited to methoxy, ethoxy, n-propoxy, 1-propoxy, n-butoxy, and t-butoxy. An alkoxy group can be unsubstituted or substituted with one or more of the following groups: halogen, hydroxyl, C₁-C₆alkoxy, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —O(C₁-C₆alkyl), —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆carboxyamidoalkyl-, or —NO₂.

“(Alkoxy)carbonyl” refers to the group alkyl-O—C(O)—. An ((alkoxy)carbonyl group can be unsubstituted or substituted with one or more of the following groups: halogen, hydroxyl, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —O(C₁-C₆alkyl), —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆-carboxyamidoalkyl-, or —NO₂. Exemplary (C₁-C₆alkoxy)carbonyl groups include but are not limited to CH₃—O—C(O)—, CH₃CH₂—O—C(O)—, CH₃CH₂CH₂—O—C(O)—, (CH₃)₂CH—O—C(O)—, and CH₃CH₂CH₂CH₂—O—C(O)—.

“Alkyl” refers to a hydrocarbon chain that may be a straight chain or branched chain, containing the indicated number of carbon atoms e.g. of 1-10 carbon atoms, 1-6 carbon atoms, or 1-4 carbon atoms. For example, C₁-C₁₀ indicates that the group may have from 1 to 10 (inclusive) carbon atoms in it. In the absence of any numerical designation, “alkyl” is a chain (straight or branched) having 1 to 6 (inclusive) carbon atoms in it. Examples of C₁-C₆alkyl groups include, but are not limited to, methyl, ethyl, propyl, butyl, pentyl, hexyl, isopropyl, isobutyl, sec-butyl, tert-butyl, isopentyl, neopentyl, and isohexyl. An alkyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆-carboxyamidoalkyl-, or —NO₂.

The carbon number as used in the definitions herein refers to carbon backbone and carbon branching, but does not include carbon atoms of the substituents, such as alkoxy substitutions and the like.

“(Alkyl)carboxyamido-” refers to an —NHC(O)— group in which the carbonyl carbon atom of said group is attached to an alkyl group, as defined above. Representative examples of a (C₁-C₆alkyl)carboxyamido group include, but are not limited to, —NHC(O)CH₃, —NHC(O)CH₂CH₃, —NHC(O)CH₂CH₂CH₃, —NHC(O)CH₂CH₂CH₂CH₃, —NHC(O)CH₂CH₂CH₂CH₂CH₃, —NHC(O)CH(CH₃)₂, —NHC(O)CH₂CH(CH₃)₂, —NHC(O)CH(CH₃)CH₂CH₃, —NHC(O)—C(CH₃)₃ and —NHC(O)CH₂C(CH₃)₃.

“(Alkyl)amino-” refers to an —NH group, the nitrogen atom of said group being attached to an alkyl group, as defined above. Representative examples of an (C₁-C₆alkyl)amino group include, but are not limited to —NHCH₃, —NHCH₂CH₃, —NHCH₂CH₂CH₃, —NHCH₂CH₂CH₂CH₃, —NHCH(CH₃)₂, —NHCH₂CH(CH₃)₂, —NHCH(CH₃)CH₂CH₃ and —NH—C(CH₃)₃. An (alkyl)amino group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆-carboxyamidoalkyl-, or —NO₂.

“(Alkyl)N-alkylamido-” refers to a —C(O)NH— group in which the nitrogen atom of said group is attached to an alkyl group, as defined above. Representative examples of a (C₁-C₆alkyl)N-alkylamido group include, but are not limited to, —C(O)NHCH₃, —C(O)NHCH₂CH₃, —C(O)NHCH₂CH₂CH₃, —C(O)NHCH₂CH₂CH₂CH₃, —C(O)NHCH₂CH₂CH₂CH₂CH₃, —C(O)NHCH(CH₃)₂, —C(O)NHCH₂CH(CH₃)₂, —C(O)NHCH(CH₃)CH₂CH₃, —C(O)NH—C(CH₃)₃ and —C(O)NHCH₂C(CH₃)₃.

“Alkylcarboxy” refers to carbon atoms of a straight or branched chain, e.g. of 1-10 carbon atoms, 1-6 carbon atoms, or 1-4 carbon atoms, attached to the parent structure through the oxygen atom of a carboxyl (C(O)—O—) functionality. Examples of C₁-C₆alkylcarboxy include acetoxy, ethylcarboxy, propylcarboxy, and isopentylcarboxy.

“Alkylene”, “alkenylene”, and “alkynylene”—are other subsets of alkyl, alkenyl and alkynyl, as defined above, including the same residues as alkyl, alkenyl, and alkynyl, but having two points of attachment within a chemical structure. Examples of alkylene include ethylene (—CH₂CH₂—), propylene (—CH₂CH₂CH₂—), and dimethylpropylene (—CH₂C(CH₃)₂CH₂—). Likewise, examples of alkenylene include ethenylene (—CH═CH— and propenylene (—CH═CH—CH₂—). Examples of alkynylene include ethynylene (—C≡C—) and propynylene (—C≡C—CH₂—).

“Alkylthio” refers to groups of straight chain or branched chain with 1 to 6 carbon atoms, attached to the parent structure through a sulfur atom. Examples of a C₁-C₆alkylthio group include methylthio, ethylthio, n-propylthio, i-propylthio, n-butylthio, i-butylthio, s-butylthio, t-butylthio, n-pentylthio and n-hexylthio.

“Alkynyl” refers to a straight or branched chain unsaturated hydrocarbon and at least one triple bond e.g. of 2-10 carbon atoms, 2-6 carbon atoms, or 2-4 carbon atoms. Examples of a C₂-C₁₀ alkynyl group include, but are not limited to, acetylene, propyne, 1-butyne, 2-butyne, isobutyne, sec-butyne, 1-pentyne, 2-pentyne, isopentyne, 1-hexyne, 2-hexyne, 3-hexyne, isohexyne, 1-heptyne, 2-heptyne, 3-heptyne, 1-octyne, 2-octyne, 3-octyne, 4-octyne, 1-nonyne, 2-nonyne, 3-nonyne, 4-nonyne, 1-decyne, 2-decyne, 3-decyne, 4-decyne and 5-decyne. A alkynyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

“Amidoaryl” refers to an aryl group, as defined above, wherein one of the aryl group's hydrogen atoms has been replaced with one or more —C(O)NH₂ groups. Representative examples of an amidoaryl group include 2-C(O)NH₂-phenyl, 3-C(O)NH₂-phenyl, 4-C(O)NH₂-phenyl, 1-C(O)NH₂-naphthyl, and 2-C(O)NH₂-naphthyl.

“Amino(alkyl)-” refers to an alkyl group, as defined above, wherein one or more of the alkyl group's hydrogen atoms has been replaced with —NH₂. Representative examples of an amino(C₁-C₆alkyl) group include, but are not limited to —CH₂NH₂, —CH₂CH₂NH₂, —CH₂CH₂CH₂ NH₂, —CH₂CH₂CH₂CH₂NH₂, —CH₂CH(NH₂)CH₃, —CH₂CH(NH₂)CH₂CH₃, —CH(NH₂)CH₂CH₃ and —C(CH₃)₂(CH₂NH₂), —CH₂CH₂CH₂CH₂CH₂NH₂, and —CH₂CH₂CH(NH₂)CH₂CH₃. An amino(alkyl) group can be unsubstituted or substituted with one or two of the following groups C₁-C₆alkoxy, C₆-C₁₄aryl, C₁-C₉heteroaryl, C₃-C₈cycloalkyl, and C₁-C₆alkyl.

“Aryl” refers to an aromatic hydrocarbon group. If not otherwise specified, in this specification the term aryl refers to a C₆-C₁₄aryl group. Examples of an C₆-C₁₄aryl group include, but are not limited to, phenyl, 1-naphthyl, 2-naphthyl, 3-biphen-1-yl, anthryl, tetrahydronaphthyl, fluorenyl, indanyl, biphenylenyl, and acenaphthenyl, groups. An aryl group can be unsubstituted or substituted with one or more of the following groups: C₁-C₆alkyl, C₃-C₈cycloalkyl, C₁-C₆ perfluoroalkyl-, halo, haloalkyl-, hydroxyl, C₁-C₆hydroxylalkyl-, —NH₂, aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“Arylamino” refers to a radical of formula aryl-NH—, wherein “aryl” is as defined above. Examples of C₆-C₁₄arylamino radicals include, but are not limited to, phenylamino (anilido), 1-naphthlamino, 2-naphthlamino and the like. An arylamino group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

“Aryloxy” refers to the group Ar—O— where Ar is an aryl group, as defined above. Exemplary C₆-C₁₄aryloxy groups include but are not limited to phenyloxy, α-naphthyloxy, and β-naphthyloxy. An aryloxy group can be unsubstituted or substituted with one or more of the following groups: C₁-C₅alkyl, halo, halo(C₁-C₆alkyl)-, hydroxyl, C₁-C₅hydroxylalkyl, —NH₂, -amino(C₁-C₆alkyl), -di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)amido-, or —NO₂.

“(C₆-C₁₄Aryl)alkyl” refers to a C₁-C₅alkyl group, as defined above, wherein one or more of the C₁-C₅alkyl group's hydrogen atoms has been replaced with an aryl group as defined above. (C₆-C₁₄Aryl)alkyl moieties include benzyl, 1-phenylethyl, 2-phenylethyl, 3-phenylpropyl, 2-phenylpropyl, 1-naphthylmethyl, 2-naphthylmethyl and the like. An (C₆-C₁₄aryl)alkyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

“(Aryl)alkyl” refers to an alkyl group, as defined above, wherein one or more of the alkyl group's hydrogen atoms has been replaced with an C₆-C₁₄aryl group as defined above. (C₆-C₁₄Aryl)alkyl moieties include benzyl, 1-phenylethyl, 2-phenylethyl, 3-phenylpropyl, 2-phenylpropyl, 1-naphthylmethyl, 2-naphthylmethyl and the like. An (aryl)alkyl group can be unsubstituted or substituted with one or more of the following groups: halogen, —NH₂, hydroxyl, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆carboxyamidoalkyl-, or —NO₂.

“Bicyclic cycloalkyl” refers to a bicyclic, saturated hydrocarbon ring containing 6-10 carbon atoms. Representative examples of a C₆-C₁₀bicyclic cycloalkyl include, but are not limited to, cis-1-decalinyl, trans 2-decalinyl, cis-4-perhydroindanyl, and trans-7-perhydroindanyl. A bicyclic cycloalkyl can be unsubstituted or independently substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl, haloalkyl-, aminoalkyl-, —OC(O)(C₁-C₆alkyl), C₁-C₆carboxyamidoalkyl-, or —NO₂. Additionally, each of any two hydrogen atoms on the same carbon atom of the bicyclic cycloalkyl rings can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

“Carboxyamidoalkyl-” refers to a primary carboxyamide (—CONH₂), a secondary carboxyamide (CONHR′) or a tertiary carboxyamide (CONR′R″), where R′ and R″ are the same or different substituent groups selected from C₁-C₆alkyl, C₂-C₆alkenyl, C₂-C₆alkynyl, C₆-C₁₄aryl, C₁-C₉heteroaryl, or C₃-C₈cycloalkyl, attached to the parent compound through the C(O) group by an alkylene group as defined above. Exemplary C₁-C₆carboxyamidoalkyl- groups include but are not limited to NH₂C(O)—CH₂—, CH₃NHC(O)—CH₂CH₂—, (CH₃)₂NC(O)—CH₂CH₂CH₂—, CH₂═CHCH₂NHC(O)—CH₂CH₂CH₂CH₂—, HCCCH₂NHC(O)—CH₂CH₂CH₂CH₂CH₂—, C₆H₅NHC(O)—CH₂CH₂CH₂CH₂CH₂CH₂—, 3-pyridylNHC(O)—CH₂CH(CH₃)CH₂CH₂—, and cyclopropyl-CH₂NHC(O)—CH₂CH₂C(CH₃)₂CH₂—.

“Cycloalkenyl” refers to monocyclic, non-aromatic carbocyclic rings with one or more carbon-to-carbon double bonds within the ring system e.g. of 3-10 carbon atoms, 3-8 carbon atoms, or 3-6 carbon atoms. The “cycloalkenyl” may be a single ring or may be multi-ring. Multi-ring structures may be bridged or fused ring structures. A cycloalkenyl can be unsubstituted or independently substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl, haloalkyl-, aminoalkyl-, —OC(O)(C₁-C₆alkyl), C₁-C₆carboxyamidoalkyl-, or —NO₂ Additionally, each of any two hydrogen atoms on the same carbon atom of the cycloalkenyl rings may be replaced by an oxygen atom to form an oxo (═O) substituent or the two hydrogen atoms may be replaced by an alkylenedioxy group so that the alkylenedioxy group, when taken together with the carbon atom to which it is attached, form a 5- to 7-membered heterocycle containing two oxygen atoms. Examples of C₃-C₁₀cycloalkenyls include, but are not limited to, cyclopropenyl, cyclobutenyl, cyclopentenyl, cyclohexenyl, 4,4a-octalin-3-yl, and cyclooctenyl.

“Di(alkyl)amino-” refers to a nitrogen atom which has attached to it two alkyl groups, as defined above. Each alkyl group can be independently selected from the alkyl groups. Representative examples of an di(C₁-C₆alkyl)amino- group include, but are not limited to, —N(CH₃)₂, —N(CH₂CH₃)(CH₃), —N(CH₂CH₃)₂, —N(CH₂CH₂CH₃)₂, —N(CH₂CH₂CH₂CH₃)₂, —N(CH(CH₃)₂)₂, —N(CH(CH₃)₂)(CH₃), —N(CH₂CH(CH₃)₂)₂, —NH(CH(CH₃)CH₂CH₃)₂, —N(C(CH₃)₃)₂, —N(C(CH₃)₃)(CH₃), and —N(CH₃)(CH₂CH₃). The two alkyl groups on the nitrogen atom, when taken together with the nitrogen to which they are attached, form a 3- to 7-membered nitrogen containing heterocycle wherein up to two of the carbon atoms of the heterocycle can be replaced with —N(R)—, —O—, or —S(O)ₒ—. R is hydrogen, C₁-C₆alkyl, C₃-C₈cycloalkyl, C₆-C₁₄aryl, C₁-C₉heteroaryl, amino(C₁-C₆alkyl), or C₆-C₁₄arylamino. Variable o is 0, 1, or 2.

“Halo” is —F, —Cl, —Br or —I.

“Haloalkyl” refers to an alkyl group, as defined above, wherein one or more of the C₁-C₆alkyl group's hydrogen atoms has been replaced with —F, —Cl, —Br, or —I. Each substitution can be independently selected from —F, —Cl, —Br, or —I. Representative examples of an C₁-C₆haloalkyl group include, but are not limited to —CH₂F, —CCl₃, —CF₃, CH₂CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂ CH₂CH₂ I, —CH₂CH₂CH₂CH₂CH₂Br, —CH₂CH₂CH₂CH₂CH₂I, —CH₂CH(Br)CH₃, —CH₂ CH(Cl)CH₂CH₃, —CH(F)CH₂CH₃ and —C(CH₃)₂(CH₂Cl).

“Heteroaryl” refers to 5-10-membered mono and bicyclic aromatic groups containing at least one heteroatom selected from oxygen, sulfur and nitrogen. At least one of the rings of a bicyclic group is aromatic. Examples of monocyclic C₁-C₅heteroaryl radicals include, but are not limited to, oxazinyl, thiazinyl, diazinyl, triazinyl, tetrazinyl, imidazolyl, tetrazolyl, isoxazolyl, furanyl, furazanyl, oxazolyl, thiazolyl, thiophenyl, pyrazolyl, triazolyl, pyrimidinyl, N-pyridyl, 2-pyridyl, 3-pyridyl and 4-pyridyl. Examples of C₁-C₉bicyclic heteroaryl radicals include but are not limited to, benzimidazolyl, indolyl, indolinyl, isoquinolinyl, 5,6,7,8-tetrahydroquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl. A heteroaryl group can be unsubstituted or substituted with one or more of the following groups: C₁-C₆alkyl, halo, haloalkyl-, hydroxyl, C₁-C₆hydroxylalkyl-, —NH₂, aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“(Heteroaryl)oxy” refers to the group Het-O— where Het is a heteroaryl group, as defined above. Exemplary (C₁-C₉heteroaryl)oxy groups include but are not limited to pyridin-2-yloxy, pyridin-3-yloxy, pyrimidin-4-yloxy, and oxazol-5-yloxy. A (heteroaryl)oxy group can be unsubstituted or substituted with one or more of the following groups: C₁-C₆alkyl, halo, haloalkyl-, hydroxyl, C₁-C₆hydroxylalkyl-, —NH₂, aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

The term “heteroatom” as used herein designates a sulfur, nitrogen, or oxygen atom.

“Hydroxylalkyl-” refers to an alkyl group, as defined above, wherein one or more of the alkyl group's hydrogen atoms has been replaced with hydroxyl groups. Examples of C₁-C₆hydroxylalkyl- moieties include, for example, —CH₂OH, —CH₂CH₂OH, —CH₂CH₂CH₂OH, —CH₂CH(OH)CH₂OH, —CH₂CH(OH)CH₃, —CH(CH₃)CH₂OH and higher homologs.

“Monocyclic heterocycle” refers to a monocyclic aromatic, cycloalkyl, or cycloalkenyl in which 1-4 of the ring carbon atoms have been independently replaced with an N, O or S atom. The monocyclic heterocyclic ring can be attached via a nitrogen, sulfur, or carbon atom. Representative examples of a 3- to 7-membered monocyclic heterocycle group include, but are not limited to, piperidinyl, 1,2,5,6-tetrahydropyridinyl, piperazinyl, morpholinyl, pyrrolyl, oxazinyl, thiazinyl, diazinyl, triazinyl, tetrazinyl, imidazolyl, tetrazolyl, pyrrolidinyl, isoxazolyl, furanyl, furazanyl, pyridinyl, oxazolyl, thiazolyl, thiophenyl, pyrazolyl, triazolyl, and pyrimidinyl. A 3- to 7-membered monocyclic heterocycle group can be unsubstituted or substituted with one or more of the following groups: C₁-C₈acyl, C₁-C₆alkyl, C₁-C₆heterocyclylalkyl, (C₆-C₁₄aryl)alkyl, halo, C₁-C₆haloalkyl-, hydroxyl, C₁-C₆hydroxylalkyl-, —NH₂, aminoalkyl-, -dialkylamino-, —COOH, —C(O)O—(C₁-C₆alkyl), —OC(O)(C₁-C₆alkyl), (C₆-C₁₄aryl)alkyl-O—C(O)—, N-alkylamido-, —C(O)NH₂, (C₁-C₆alkyl)amido-, or —NO₂.

“Bicyclic heterocycle” refers to a bicyclic aromatic, bicyclic cycloalkyl, or bicyclic cycloalkenyl in which 1-4 of the ring carbon atoms have been independently replaced with an N, O or S atom. Representative examples of a 6- to 10-membered bicyclic heterocycle group include, but are not limited to, benzimidazolyl, indolyl, indolinyl, isoquinolinyl, indazolyl, quinolinyl, tetrahydroquinolinyl, quinazolinyl, purinyl, benzisoxazolyl, benzoxazolyl, benzthiazolyl, benzodiazolyl, benzotriazolyl, isoindolyl and indazolyl. A 6- to 10-membered bicyclic heterocycle group can be unsubstituted or substituted with one or more of the following groups: C₁-C₈acyl, C₁-C₆alkyl, C₁-C₆heterocyclylalkyl, (C₆-C₁₄aryl)alkyl, halo, C₁-C₆haloalkyl-, hydroxyl, C₁-C₆hydroxylalkyl-, —NH₂, aminoalkyl-, -dialkylamino-, —COOH, —C(O)O—(C₁-C₆alkyl), —OC(O)(C₁-C₆alkyl), (C₆-C₁₄aryl)alkyl-O—C(O)—, N-alkylamido-, —C(O)NH₂, (C₁-C₆alkyl)amido-, or —NO₂.

“Heterocyclyl(alkyl)-” refers to an alkyl group, as defined above, wherein one or more of the alkyl group's hydrogen atoms has been replaced with a heterocycle group as defined above. Heterocyclyl(C₁-C₆alkyl)- moieties include 2-pyridylmethyl, 1-piperazinylethyl, 4-morpholinylpropyl, 6-piperazinylhexyl, and the like. A heterocyclyl(alkyl) group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carboxyamido-, HC(O)NH—, H₂NC(O)—, (C₁-C₆alkyl)NHC(O)—, di(C₁-C₆alkyl)NC(O)—, NC—, hydroxyl, C₁-C₆alkoxy-, C₁-C₆alkyl-, HO₂C—, (C₁-C₆alkoxy)carbonyl-, (C₁-C₆alkyl)C(O)—, 4- to 7-membered monocyclic heterocycle, C₆-C₁₄aryl-, C₁-C₉heteroaryl-, or C₃-C₈cycloalkyl-.

“Leaving group” refers an atom or group (charged or uncharged) that becomes detached from an atom in what is considered to be the residual or main part of the substrate in a specified reaction. For example, in the heterolytic solvolysis of benzyl bromide in acetic acid: the leaving group is bromide. In the reaction of N,N,N-trimethyl-1-phenylmethanaminium ion with methanethiolate, the leaving group is trimethylamine. In the electrophilic nitration of benzene, it is H⁺. The term has meaning only in relation to a specified reaction. Examples of leaving groups include, for example, carboxylates (i.e. CH₃COO⁻, CF₃CO₂⁻), F⁻, water, Cl⁻, Br⁻, I⁻, N₃⁻, SCN⁻, trichloroacetimidate, thiopyridyl, tertiary amines (i.e. trimethylamine), phenoxides (i.e. nitrophenoxide), and sulfonates (i.e. tosylate, mesylate, triflate).

“Perfluoroalkyl-” refers to a straight or branched chain hydrocarbon having two or more fluorine atoms. Examples of a C₁-C₆ perfluoroalkyl-group include CF₃, CH₂CF₃, CF₂CF₃ and CH(CF₃)₂.

The term “optionally substituted” as used herein means that at least one hydrogen atom of the optionally substituted group has been substituted with halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

A “subject” is a mammal, e.g., a human, mouse, rat, guinea pig, dog, cat, horse, cow, pig, or non-human primate, such as a monkey, chimpanzee, baboon or gorilla.

The compounds of the present invention do not include those that are too unstable to synthesize and/or isolate. Examples of these unstable molecules may include alkenes or alkynes with a hydroxyl, —NH—, or NH₂ group bonded to an unsaturated carbon or alkene with more than one hydroxyl group or amino group bonded to the same carbon atom.

Representative “pharmaceutically acceptable salts” include but are not limited to, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts.

An “effective amount” when used in connection a pyrrolopyrimidine compound of this invention is an amount effective for inhibiting PI3K or mTOR in a subject.

The pyrrolo[3,2-d]pyrimidine compounds of the present invention exhibit PI3K inhibitory activity and therefore, can be utilized in order to inhibit abnormal cell growth in which PI3K plays a role. Thus, the pyrrolo[3,2-d]pyrimidine compounds are effective in the treatment of disorders with which abnormal cell growth actions of PI3K are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the pyrrolo[3,2-d]pyrimidine compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

When administered to an animal, the pyrrolo[3,2-d]pyrimidine compounds or pharmaceutically acceptable salts of the pyrrolo[3,2-d]pyrimidine compounds can be administered neat or as a component of a composition that comprises a physiologically acceptable carrier or vehicle. A composition of the invention can be prepared using a method comprising admixing the pyrrolo[3,2-d]pyrimidine compound or a pharmaceutically acceptable salt of the pyrrolo[3,2-d]pyrimidine compound and a physiologically acceptable carrier, excipient, or diluent. Admixing can be accomplished using methods well known for admixing a pyrrolo[3,2-d]pyrimidine compound or a pharmaceutically acceptable salt of the pyrrolo[3,2-d]pyrimidine compound and a physiologically acceptable carrier, excipient, or diluent.

The present compositions, comprising pyrrolo[3,2-d]pyrimidine compounds or pharmaceutically acceptable salts of the pyrrolo[3,2-d]pyrimidine compounds of the invention can be administered orally. The pyrrolo[3,2-d]pyrimidine compounds or pharmaceutically acceptable salts of pyrrolo[3,2-d]pyrimidine compounds of the invention can also be administered by any other convenient route, for example, by infusion or bolus injection, by absorption through epithelial or mucocutaneous linings (e.g., oral, rectal, vaginal, and intestinal mucosa, etc.) and can be administered together with another therapeutic agent. Administration can be systemic or local. Various known delivery systems, including encapsulation in liposomes, microparticles, microcapsules, and capsules, can be used.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result of release of the pyrrolo[3,2-d]pyrimidine compound or a pharmaceutically acceptable salt of the pyrrolo[3,2-d]pyrimidine compound into the bloodstream. The mode of administration is left to the discretion of the practitioner.

In one embodiment, the pyrrolo[3,2-d]pyrimidine compound or a pharmaceutically acceptable salt of the pyrrolo[3,2-d]pyrimidine compound is administered orally.

In another embodiment, the pyrrolo[3,2-d]pyrimidine compound or a pharmaceutically acceptable salt of the pyrrolo[3,2-d]pyrimidine compound is administered intravenously.

In another embodiment, it may be desirable to administer the pyrrolo[3,2-d]pyrimidine compound or a pharmaceutically acceptable salt of the pyrrolo[3,2-d]pyrimidine compound locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, it can be desirable to introduce the pyrrolo[3,2-d]pyrimidine compound or a pharmaceutically acceptable salt of the pyrrolo[3,2-d]pyrimidine compound into the central nervous system, circulatory system or gastrointestinal tract by any suitable route, including intraventricular, intrathecal injection, paraspinal injection, epidural injection, enema, and by injection adjacent to the peripheral nerve. An intraventricular catheter, for example, can facilitate intraventricular injection attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the pyrrolo[3,2-d]pyrimidine compound or a pharmaceutically acceptable salt of the pyrrolo[3,2-d]pyrimidine compound can be formulated as a suppository, with traditional binders and excipients such as triglycerides.

In another embodiment, the pyrrolo[3,2-d]pyrimidine compound or a pharmaceutically acceptable salt of the pyrrolo[3,2-d]pyrimidine compound can be delivered in a vesicle, in particular a liposome (see Langer, Science 249:1527-1533 (1990) and Treat et al., Liposomes in the Therapy of Infectious Disease and Cancer pp. 317-327 and pp. 353-365 (1989)).

In yet another embodiment, the pyrrolo[3,2-d]pyrimidine compound or a pharmaceutically acceptable salt of the pyrrolo[3,2-d]pyrimidine compound can be delivered in a controlled-release system or sustained-release system (see, e.g., Goodson, in Medical Applications of Controlled Release, vol. 2, pp. 115-138 (1984)). Other controlled or sustained-release systems discussed in the review by Langer, Science 249:1527-1533 (1990) can be used. In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment, a controlled- or sustained-release system can be placed in proximity of a target of the pyrrolo[3,2-d]pyrimidine compound or a pharmaceutically acceptable salt of the pyrrolo[3,2-d]pyrimidine compound, e.g., the reproductive organs, thus requiring only a fraction of the systemic dose.

The present compositions can optionally comprise a suitable amount of a physiologically acceptable excipient.

Such physiologically acceptable excipients can be liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment, the physiologically acceptable excipients are sterile when administered to an animal. The physiologically acceptable excipient should be stable under the conditions of manufacture and storage and should be preserved against the contaminating action of microorganisms. Water is a particularly useful excipient when the pyrrolo[3,2-d]pyrimidine compound or a pharmaceutically acceptable salt of the pyrrolo[3,2-d]pyrimidine compound is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Liquid carriers may be used in preparing solutions, suspensions, emulsions, syrups, and elixirs. The pyrrolo[3,2-d]pyrimidine compound or pharmaceutically acceptable salt of the pyrrolo[3,2-d]pyrimidine compound of this invention can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule. Other examples of suitable physiologically acceptable excipients are described in Remington's Pharmaceutical Sciences pp. 1447-1676 (Alfonso R. Gennaro, ed., 19th ed. 1995).

In one embodiment, the pyrrolo[3,2-d]pyrimidine compound or a pharmaceutically acceptable salt of the pyrrolo[3,2-d]pyrimidine compound is formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided pyrrolo[3,2-d]pyrimidine compound or pharmaceutically acceptable salt of the pyrrolo[3,2-d]pyrimidine compound. In tablets, the pyrrolo[3,2-d]pyrimidine compound or pharmaceutically acceptable salt of the pyrrolo[3,2-d]pyrimidine compound is mixed with a carrier having the necessary compression properties in suitable proportions and compacted in the shape and size desired. The powders and tablets can contain up to about 99% of the pyrrolo[3,2-d]pyrimidine compound or pharmaceutically acceptable salt of the pyrrolo[3,2-d]pyrimidine compound.

Capsules may contain mixtures of the pyrrolo[3,2-d]pyrimidine compounds or pharmaceutically acceptable salts of the pyrrolo[3,2-d]pyrimidine compounds with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrroldine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the pyrrolo[3,2-d]pyrimidine compound or a pharmaceutically acceptable salt of the pyrrolo[3,2-d]pyrimidine compound can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the pyrrolo[3,2-d]pyrimidine compound or a pharmaceutically acceptable salt of the pyrrolo[3,2-d]pyrimidine compound is to be administered by infusion, it can be dispensed, for example, with an infusion bottle containing sterile pharmaceutical grade water or saline. Where the pyrrolo[3,2-d]pyrimidine compound or a pharmaceutically acceptable salt of the pyrrolo[3,2-d]pyrimidine compound is administered by injection, an ampoule of sterile water for injection or saline can be provided so that the ingredients can be mixed prior to administration.

In another embodiment, the pyrrolo[3,2-d]pyrimidine compound or pharmaceutically acceptable salt of the pyrrolo[3,2-d]pyrimidine compound can be administered transdermally through the use of a transdermal patch. Transdermal administrations include administrations across the surface of the body and the inner linings of the bodily passages including epithelial and mucosal tissues. Such administrations can be carried out using the present pyrrolo[3,2-d]pyrimidine compounds or pharmaceutically acceptable salts of the pyrrolo[3,2-d]pyrimidine compound s, in lotions, creams, foams, patches, suspensions, solutions, and suppositories (e.g., rectal or vaginal).

Transdermal administration can be accomplished through the use of a transdermal patch containing the pyrrolo[3,2-d]pyrimidine compound or pharmaceutically acceptable salt of the pyrrolo[3,2-d]pyrimidine compound and a carrier that is inert to the pyrrolo[3,2-d]pyrimidine compound or pharmaceutically acceptable salt of the pyrrolo[3,2-d]pyrimidine compound, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the pyrrolo[3,2-d]pyrimidine compound or pharmaceutically acceptable salt of the pyrrolo[3,2-d]pyrimidine compound into the blood stream, such as a semi-permeable membrane covering a reservoir containing the pyrrolo[3,2-d]pyrimidine compound or pharmaceutically acceptable salt of the pyrrolo[3,2-d]pyrimidine compound with or without a carrier, or a matrix containing the active ingredient.

The pyrrolo[3,2-d]pyrimidine compounds or pharmaceutically acceptable salts of the pyrrolo[3,2-d]pyrimidine compounds of the invention may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

The pyrrolo[3,2-d]pyrimidine compound or a pharmaceutically acceptable salt of the pyrrolo[3,2-d]pyrimidine compound can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release. Advantages of controlled- or sustained-release compositions include extended activity of the drug, reduced dosage frequency, and increased compliance by the animal being treated. In addition, controlled- or sustained-release compositions can favorably affect the time of onset of action or other characteristics, such as blood levels of the pyrrolo[3,2-d]pyrimidine compound or a pharmaceutically acceptable salt of the pyrrolo[3,2-d]pyrimidine compound, and can thus reduce the occurrence of adverse side effects.

Controlled- or sustained-release compositions can initially release an amount of the pyrrolo[3,2-d]pyrimidine compound or a pharmaceutically acceptable salt of the pyrrolo[3,2-d]pyrimidine compound that promptly produces the desired therapeutic or prophylactic effect, and gradually and continually release other amounts of the pyrrolo[3,2-d]pyrimidine compound or a pharmaceutically acceptable salt of the pyrrolo[3,2-d]pyrimidine compound to maintain this level of therapeutic or prophylactic effect over an extended period of time. To maintain a constant level of the pyrrolo[3,2-d]pyrimidine compound or a pharmaceutically acceptable salt of the pyrrolo[3,2-d]pyrimidine compound in the body, the pyrrolo[3,2-d]pyrimidine compound or a pharmaceutically acceptable salt of the pyrrolo[3,2-d]pyrimidine compound can be released from the dosage form at a rate that will replace the amount of the pyrrolo[3,2-d]pyrimidine compound or a pharmaceutically acceptable salt of the pyrrolo[3,2-d]pyrimidine compound being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or pyrrolo[3,2-d]pyrimidine compounds.

In certain embodiments, the present invention is directed to prodrugs of the pyrrolo[3,2-d]pyrimidine compounds or pharmaceutically acceptable salts of pyrrolo[3,2-d]pyrimidine compounds of the present invention. Various forms of prodrugs are known in the art, for example as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975).

The amount of the pyrrolo[3,2-d]pyrimidine compound or a pharmaceutically acceptable salt of the pyrrolo[3,2-d]pyrimidine compound that is effective for treating or preventing a PI3K-related disorder. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one pyrrolo[3,2-d]pyrimidine compound or a pharmaceutically acceptable salt of the pyrrolo[3,2-d]pyrimidine compound is administered, the effective dosage amounts correspond to the total amount administered.

The amount of the pyrrolo[3,2-d]pyrimidine compound or a pharmaceutically acceptable salt of the pyrrolo[3,2-d]pyrimidine compound that is effective for treating or preventing an PI3K-related disorder will typically range from about 0.001 mg/kg to about 250 mg/kg of body weight per day, in one embodiment, from about 1 mg/kg to about 250 mg/kg body weight per day, in another embodiment, from about 1 mg/kg to about 50 mg/kg body weight per day, and in another embodiment, from about 1 mg/kg to about 20 mg/kg of body weight per day.

In one embodiment, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may be given in a single dose or in two or more divided doses.

The pyrrolo[3,2-d]pyrimidine compound or a pharmaceutically acceptable salt of the pyrrolo[3,2-d]pyrimidine compound can be assayed in vitro or in vivo for the desired therapeutic or prophylactic activity prior to use in humans. Animal model systems can be used to demonstrate safety and efficacy.

The present methods for treating or preventing an PI3K-related disorder, can further comprise administering another therapeutic agent to the animal being administered the pyrrolo[3,2-d]pyrimidine compound or a pharmaceutically acceptable salt of the pyrrolo[3,2-d]pyrimidine compound. In one embodiment, the other therapeutic agent is administered in an effective amount.

Effective amounts of the other therapeutic agents are well known to those skilled in the art. However, it is well within the skilled artisan's purview to determine the other therapeutic agent's optimal effective amount range. The pyrrolo[3,2-d]pyrimidine compound or a pharmaceutically acceptable salt of the pyrrolo[3,2-d]pyrimidine compound and the other therapeutic agent can act additively or, in one embodiment, synergistically. In one embodiment, of the invention, where another therapeutic agent is administered to an animal, the effective amount of the pyrrolo[3,2-d]pyrimidine compound or a pharmaceutically acceptable salt of the pyrrolo[3,2-d]pyrimidine compound is less than its effective amount would be where the other therapeutic agent is not administered. In this case, without being bound by theory, it is believed that the pyrrolo[3,2-d]pyrimidine compound or a pharmaceutically acceptable salt of the pyrrolo[3,2-d]pyrimidine compound and the other therapeutic agent act synergistically.

Suitable other therapeutic agents useful in the methods and compositions of the present invention include, but are not limited to temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins herbimycin A, genistein, erbstatin, and lavendustin A.

Other therapeutic agents useful in the methods and compositions of the present invention include, but are not limited to hydroxyzine, glatiramer acetate, interferon beta-1a, interferon beta-1b, mitoxantrone, and natalizumab.

In one embodiment, the pyrrolo[3,2-d]pyrimidine compound or a pharmaceutically acceptable salt of the pyrrolo[3,2-d]pyrimidine compound is administered concurrently with another therapeutic agent.

In one embodiment, a composition comprising an effective amount of the pyrrolo[3,2-d]pyrimidine compound or a pharmaceutically acceptable salt of the pyrrolo[3,2-d]pyrimidine compound and an effective amount of another therapeutic agent within the same composition can be administered. In another embodiment, a composition comprising an effective amount of the pyrrolo[3,2-d]pyrimidine compound or a pharmaceutically acceptable salt of the pyrrolo[3,2-d]pyrimidine compound and a separate composition comprising an effective amount of another therapeutic agent can be concurrently administered. In another embodiment, an effective amount of the pyrrolo[3,2-d]pyrimidine compound or a pharmaceutically acceptable salt of the pyrrolo[3,2-d]pyrimidine compounds administered prior to or subsequent to administration of an effective amount of another therapeutic agent. In this embodiment, the pyrrolo[3,2-d]pyrimidine compound or a pharmaceutically acceptable salt of the pyrrolo[3,2-d]pyrimidine compounds administered while the other therapeutic agent exerts its therapeutic effect, or the other therapeutic agent is administered while the pyrrolo[3,2-d]pyrimidine compound or a pharmaceutically acceptable salt of the pyrrolo[3,2-d]pyrimidine compound exerts its preventative or therapeutic effect for treating or preventing an PI3K-related disorder.

In another embodiment, the pharmaceutically acceptable carrier is suitable for oral administration and the composition comprises an oral dosage form.

Methods useful for making the pyrrolo[3,2-d]pyrimidine compounds are set forth in the Examples below and generalized in Schemes 1-8:

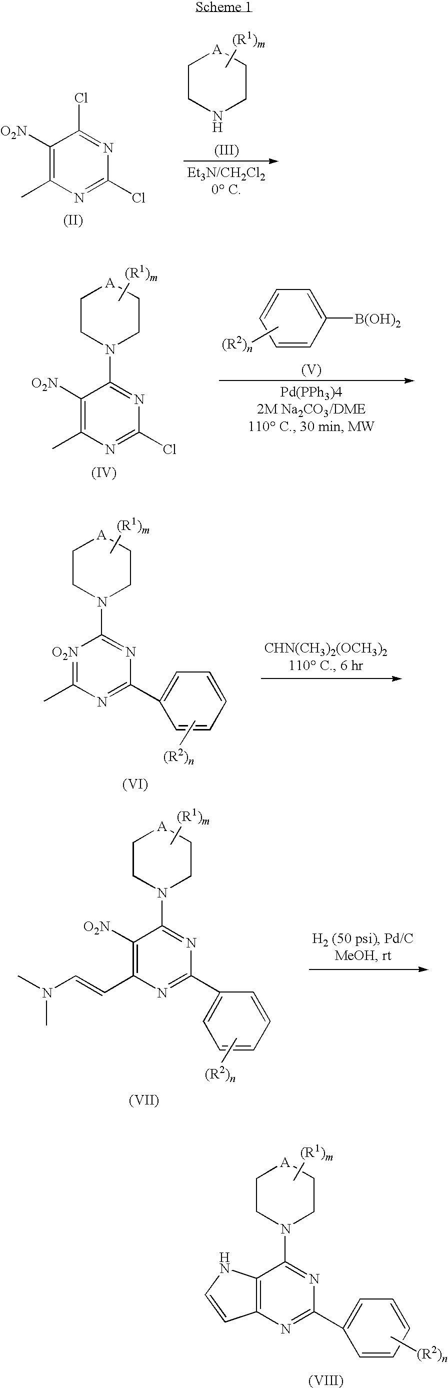

4-Morpholino-2-aryl-5H-pyrrolo[3,2-d]pyrimidine compounds were prepared by a four-step sequence as depicted in Scheme 1. The commercially available 6-methyl-5-nitro-2,4-dichloropyrimidine (II) was reacted with a morpholine (III) and the resulting product (IV) was subjected to Suzuki reaction with different boronic acids(V) or esters under microwave conditions or under thermal conditions to give the corresponding 4-morpholino-2-arylpyrimidine intermediates (VI). The pyrrole ring was formed by reacting the 4-morpholino-2-Arylpyrimidine intermediate with 1,1-dimethoxy-N,N-dimethylmethylamine (DMFDMA) to give the corresponding enamine (VII), which subsequently was cyclized under catalytic hydrogenation to give the desired 4-Morpholino-2-aryl-5H-pyrrolo[3,2-d]pyrimidine compounds. (VIII)).

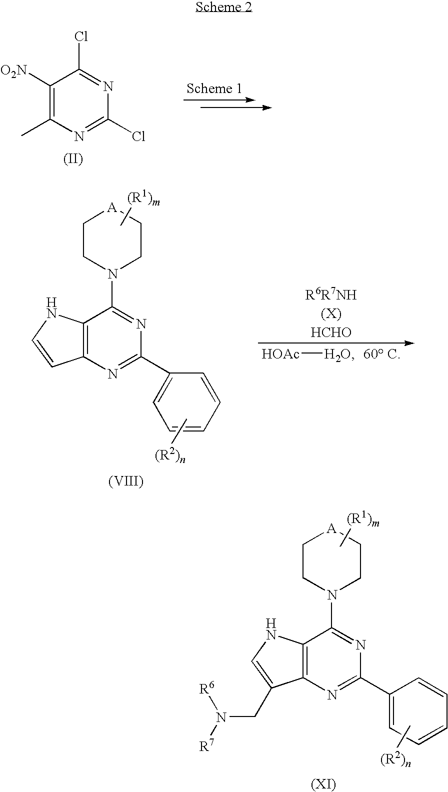

4-Morpholino-2-aryl-7-aminomethyl-5H-pyrrolo[3,2-d]pyrimidine compounds were prepared by a Mannich reaction of the 4-morpholino-2-aryl-5H-pyrrolo[3,2-d]pyrimidine compounds (VIII) (prepared as Scheme 1) with formaldehyde and different amines (X) under acidic conditions (Scheme 2).

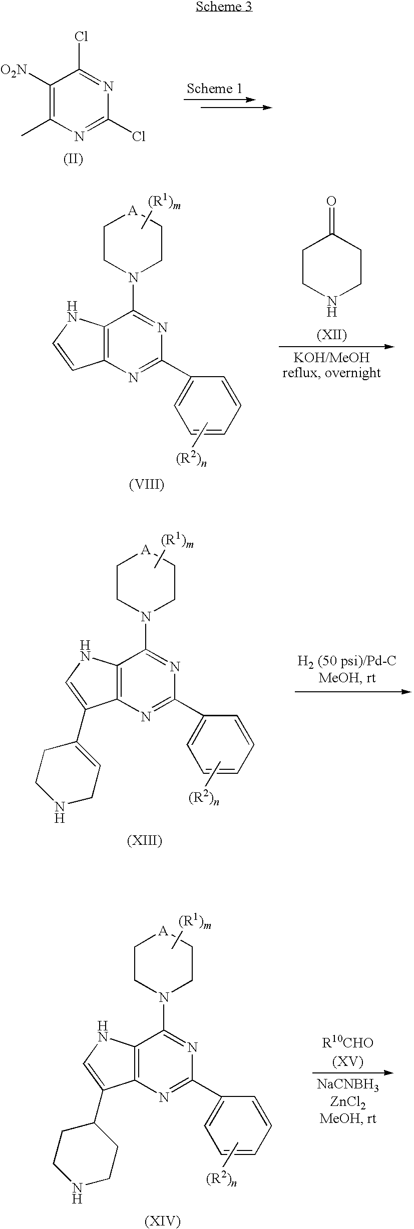

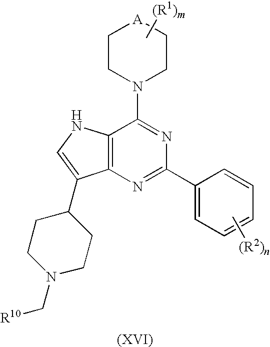

4-Morpholino-2-aryl-7-substituted piperidin-4-yl-5H-pyrrolo[3,2-d]pyrimidine compounds were prepared by a three-step sequence as illustrated in Scheme 3. The pyrrolopyrimidine compounds (VIII), obtained by the Scheme 1, were heated with piperidin-4-one (XII) under basic condition to form an adol type adduct (XIII). The resultant products were reduced by catalytic hydrogenation. Finally, reductive amination with different aldehydes (XV) or ketones yielded the desired products (XVI).

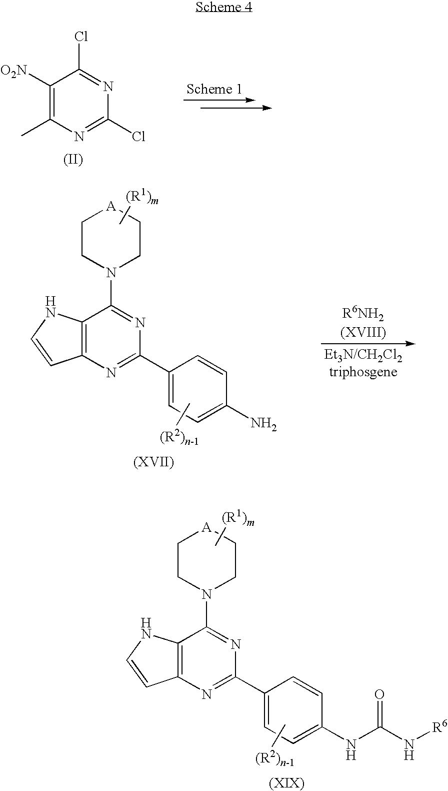

Urea analogs of pyrrolopyrimidine compounds were prepared according to Scheme 4. The intermediate 3-(4-morpholino-5H-pyrrolo[3,2-d]pyrimidin-2-yl)aniline, (XVII) prepared according to Scheme 1, was reacted with triphosgene in the presence of Et₃N, followed by reacting with different amines (XVIII) to give the desired urea compounds (XIX).

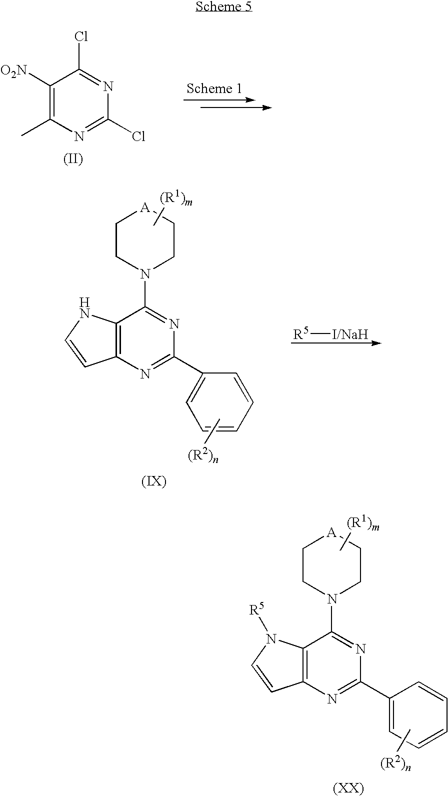

4-Morpholino-2-aryl-5-substituted-5H-pyrrolo[3,2-d]pyrimidine compounds (XX) were prepared according to Scheme 5. [3-(4-Morpholin-4-yl-5H-pyrrolo[3,2-d]pyrimidin-2-yl)phenyl] (VIII), prepared according to the general method described in Scheme 1, was alkylated at the pyrrole nitrogen by treating with sodium hydride and an alkylating agent such as iodomethane.

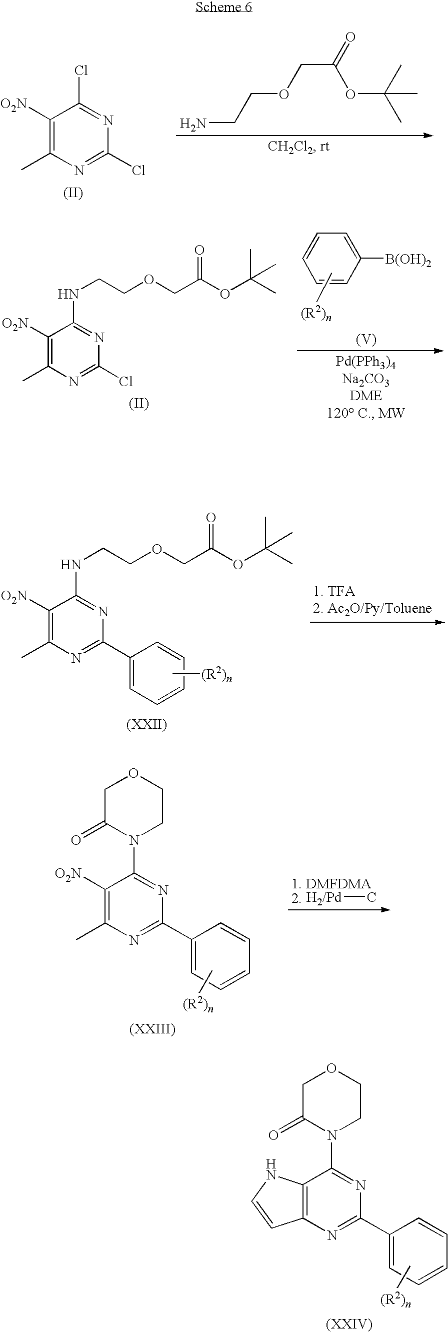

4-(3-Oxomorpholino)-2-aryl-5H-pyrrolo[3,2-d]pyrimidine compounds (XXIV) were prepared according to Scheme 6 by a six-step sequence. 2,4-dichloro-6-methyl-5-nitropyrimidine (II) was reacted with tert-butyl (2-amino-ethoxy)acetate in the presence of Et₃N, followed by Suzuki coupling with boronic acid (V) to give the intermediate tert-butyl [2-({2-[3-(benzyloxy)phenyl]-6-methyl-5-nitropyrimidin-4-yl}amino)ethoxy]acetate, (XXII), which was converted to the acid by treating with TFA. Lactam formation was achieved by heating the acid with acetic anhydride and 1 equivalent of pyridine in toluene to give 4-{2-[3-(benzyloxy)phenyl]-6-methyl-5-nitropyrimidin-4-yl}morpholin-3-one (XXIII). According to established method (Scheme 1), the desired product (XXIV) was produced by cyclization.

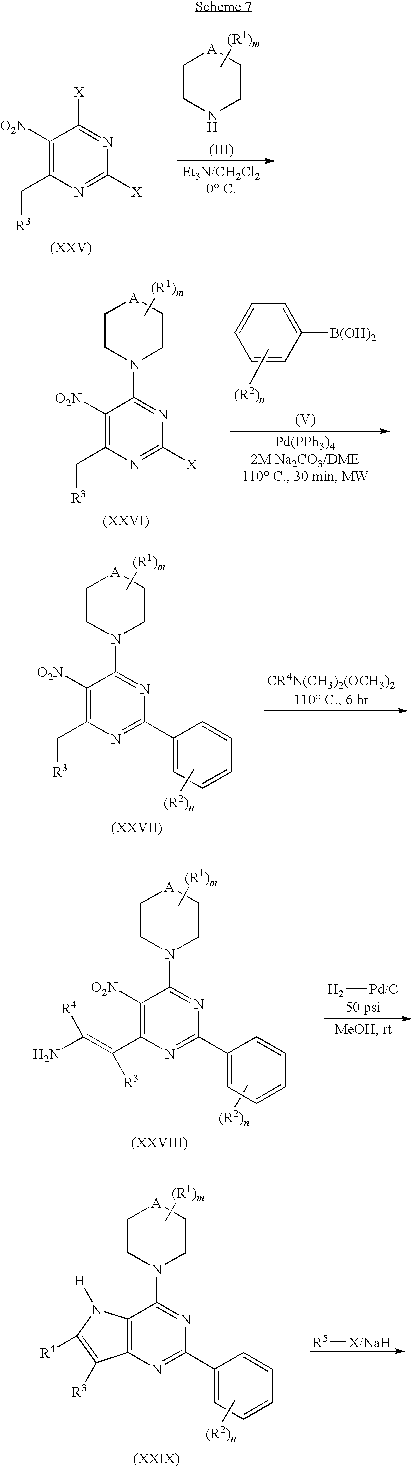

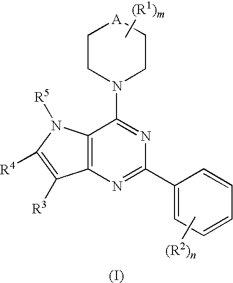

Scheme 7 shows a synthesis of (I) using an amide acetal reagent CR⁴N(CH₃)₂(OCH₃)₂.

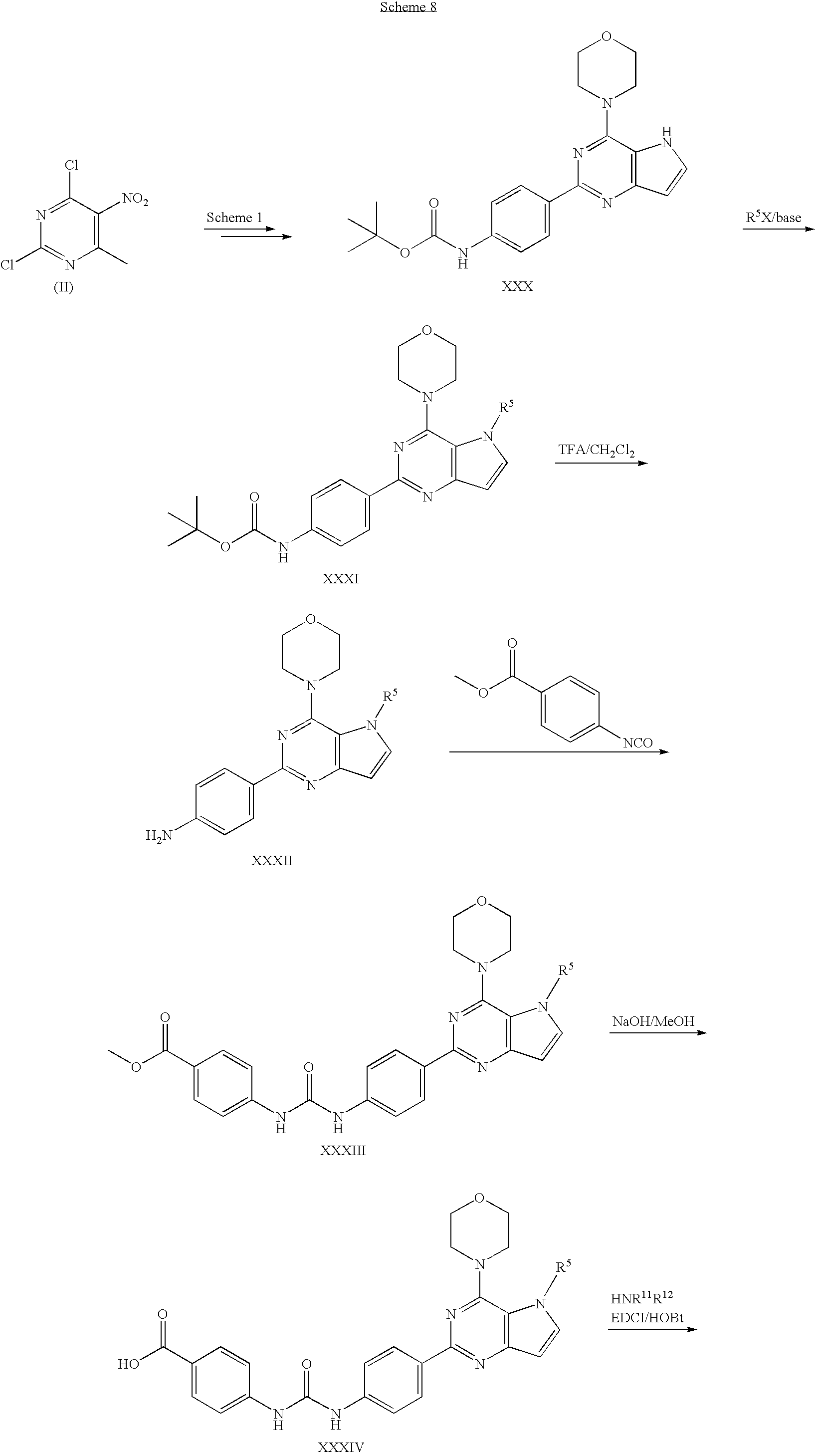

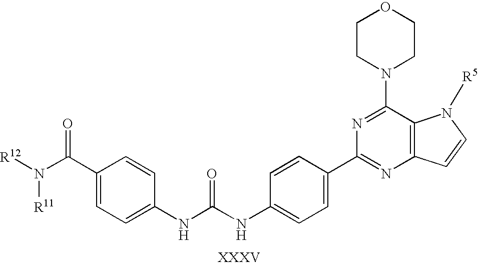

Scheme 8 shows elaboration of a urea side chain as substituent R².

One of skill in the art will recognize that Schemes 1-8 can be adapted to produce the other pyrrolopyrimidine compounds and pharmaceutically acceptable salts of pyrrolopyrimidine compounds according to the present invention.

### EXAMPLES

The following abbreviations are used herein and have the indicated definitions: ACN is acetonitrile, AcOH is acetic acid, ATP is adenosine triphosphate. Celite™ is flux-calcined diatomaceous earth. Celite™ is a registered trademark of World Minerals Inc. CHAPS is 3[(3-cholamidopropyl)dimethylammonio]-propanesulfonic acid, DEAD is diethyl azodicarboxylate, DIAD is diisopropylazodicarboxylate, DMAP is dimethyl aminopyridine, DMF is N,N-dimethylformamide, DMF-DMA is dimethylformamide dimethyl acetal, DMSO is dimethylsulfoxide, DPBS is Dulbecco's Phosphate Buffered Saline Formulation, EDCI is 1-ethyl-3-(3-dimethylaminopropyl) carbodiimide or water-soluble carbodiimide, EDTA is ethylenediaminetetraacetic acid, ESI stands for Electrospray Ionization, EtOAc is ethyl acetate, EtOH is ethanol, HEPES is 4-(2-hydroxyethyl)-1-piperazineethanesulfonic acid, Hunig's Base is diisopropylethylamine, HOBT is N-hydroxybenzotriazole, HPLC is high pressure liquid chromatography, LPS is lipopolysaccharide, MeCN is acetonitrile, MeOH is methanol, MS is mass spectrometry, NEt₃ is triethylamine, NMR is nuclear magnetic resonance, PBS is phosphate-buffered saline (pH 7.4), RPMI 1640 is a buffer (Sigma-Aldrich Corp., St. Louis, Mo., USA), SDS is dodecyl sulfate (sodium salt), SRB is Sulforhodamine B, TCA is tricholoroacetic acid, TFA is trifluoroacetic acid, THF is tetrahydrofuran, TLC is thin-layer chromatography, and TRIS is tris(hydroxymethyl)aminomethane.

The following methods outline the synthesis of the pyrrolopyrimidine compounds.

### Experimental for the Preparation of 4-Morpholino-2-Aryl-7-5H-pyrrolo[3,2-d]pyrimidine (Scheme 1)

### Example 1

### General Procedure

### Preparation of 2-[3-(benzyloxy)phenyl]-4-morpholin-4-yl-5H-pyrrolo[3,2-d]pyrimidine

Step 1: Synthesis of 4-(2-chloro-6-methyl-5-nitropyrimidin-4-yl)morpholine

To a stirred solution of 2,4-dichloro-6-methyl-5-nitro-pyrimidine (5.0 g, 24.15 mmol) in CH₂Cl₂ (50 mL) was added a solution of morpholine (2.1 mL, 24.15 mmol) in CH₂Cl₂ (20 mL), followed by the addition of triethylamine (6.7 mL, 48.3 mmol) at 0° C. The resulting mixture was stirred at room temperature overnight and diluted with CH₂Cl₂. The organic solution was washed with water and brine, and dried over MgSO₄. The solvent was removed by evaporation under reduced pressure, and the residue was purified by flash chromatography to give the titled compound as yellow solid (6.17 g, 99% yield). MS (ESI) m/z 259.0

Step 2: Synthesis of 4-{2-[3-(benzyloxy)phenyl]-6-methyl-5-nitropyrimidin-4-yl}morpholine

To a stirred solution of 4-(2-chloro-6-methyl-5-nitropyrimidin-4-yl)morpholine (400 mg, 1.55 mmol) in 8 mL of 1,2-dimethoxymethane (DME) were added 3-benzyloxyphenylboronic acid (533 mg, 2.34 mmol), Pd(Ph₃)₄ (90 mg, 5 mol %) and 2M Na₂CO₃ aqueous solution (6 mL). The resulting mixture was heated at 110° C. for 30 min in microwave oven. The reaction mixture was cooled to room temperature, filtered, and washed with THF. The filtrate was diluted with EtOAc, washed with brine, and dried over MgSO₄. The solvent was removed by evaporation under reduced pressure, and the residue was purified by flash chromatography to give the titled compound as yellow solid (600 mg, 95% yield). MS (ESI) m/z 407.3

Step 3: Synthesis of (E)-2-{2-[3-(benzyloxy)phenyl]-6-morpholin-4-yl-5-nitropyrimidin-4-yl}-N,N-dimethylethenamine

A mixture of 4-{2-[3-(benzyloxy)phenyl]-6-methyl-5-nitropyrimidin-4-yl}morpholine (600 mg, 1.48 mmol) and 20 mL of N,N-dimethylformamide dimethyl acetal (DMF-DMA) was heated at 110° C. overnight. The reaction mixture was cooled to room temperature, and concentrated in vacuum. The residue was diluted EtOAc, and filtered through a short silica gel column. The filtrate was concentrated, and the residue was treated with ether. The resulting red solid was collected by filtration to give the titled compound (641 mg, 94% yield). MS (ESI) m/z 462.3.

Step 4: Synthesis of 2-[3-(benzyloxy)phenyl]-4-morpholin-4-yl-5H-pyrrolo[3,2-d]pyrimidine

To a solution of (E)-2-{2-[3-(benzyloxy)phenyl]-6-morpholin-4-yl-5-nitropyrimidin-4-yl}-N,N-dimethylethenamine (350 mg, 0.76 mmol) in 50 mL of methanol was added 40 mg of 10% Pd/C as catalyst. The resulting mixture was taken to hydrogenation (H₂, 50 psi) at room temperature overnight. The reaction mixture was filtered through a pad of Celite™. The filtration was concentrated in vacuum, and the residue was purified by flash chromatography (EtOAc:Hexanes=80:20) to give the title compound as off-white solid (249 mg, 85% yield); MS (ESI) m/z 387.2

### Example 2

### Preparation of 3-(4-morpholin-4-yl-5H-pyrrolo[3,2-d]pyrimidin-2-yl)phenol

To a solution of 2-[3-(benzyloxy)phenyl]-4-morpholin-4-yl-5H-pyrrolo[3,2-d]pyrimidine (249 mg, 0.64 mmol) in 20 mL of methanol was added 10% Pd/C (40 mg) and acetic acid (1 mL). The resulting mixture was shaken under hydrogen (H₂, 50 psi) at room temperature overnight. The reaction mixture was filtered through a pad of Celite and the filtrate was concentrated in vacuum. The obtained residue was purified by flash column chromatography (EtOAc:Hexanes=80:20) to give the title compound as off-white solid (180 mg, 95% yield); MS (ESI) m/z 297.1

### Example 3

### Preparation of [3-(4-morpholin-4-yl-5H-pyrrolo[3,2-d]pyrimidin-2-yl)phenyl]methanol

Following the procedure as described as in Example 1 step 2, Suzuki coupling of 4-(2-chloro-6-methyl-5-nitropyrimidin-4-yl)morpholine (1.1 g, 4.26 mmol) and 3-(hydroxymethyl)phenylboronic acid (0.882 g, 6.39 mmol) gave the intermediate 3-(4-methyl-6-morpholino-5-nitropyrimidin-2-yl)phenyl)methanol (1.41 g, 99% yield, MS (ESI): m/z 331.2), which was converted to the title compound (by following the procedure as described as in Example 1 step 3 and 4) as off-white solid (Yield: 542 mg, 41%). MS (ESI) m/z 311.2

### Example 4

### Preparation of 2-(3-methylphenyl)-4-morpholin-4-yl-5H-pyrrolo[3,2-d]pyrimidine

This compound was isolated as a by-product in the preparation of [3-(4-morpholin-4-yl-5H-pyrrolo[3,2-d]pyrimidin-2-yl)phenyl]methanol (Example 3) as off-white solid (17 mg, 1.4% yield); MS (ESI) m/z 295.2

### Example 5

### Preparation of 3-(4-morpholin-4-yl-5H-pyrrolo[3,2-d]pyrimidin-2-yl)aniline

Following the procedure as described as in Example 1 step 2, Suzuki coupling of 4-(2-chloro-6-methyl-5-nitropyrimidin-4-yl)morpholine (258 mg, 1 mmol) with 3-nitro-phenylboronic acid (200 mg, 1.2 mmol) gave the intermediate 4-(6-methyl-5-nitro-2-(3-nitrophenyl)pyrimidin-4-yl)morpholine (246 mg, 71% yield, MS (ESI): m/z 346.3), which was converted to the title compound (by following the procedure as described as in Example 1 step 3 and 4) as yellow solid (Yield: 113 mg, 54%). MS (ESI) m/z 296.3

### Example 6

### Preparation of 1-methyl-3-[4-(4-morpholin-4-yl-5H-pyrrolo[3,2-d]pyrimidin-2-yl)phenyl]urea

Following the procedure as described as in Example 1 step 2, Suzuki coupling of 4-(2-chloro-6-methyl-5-nitropyrimidin-4-yl)morpholine (629 mg, 2.44 mmol) with 4-(3-methylureido)phenylboronic acid pinacol ester (prepared by reaction of (4-isocyanatophenyl)boronic acid pinacol ester (980 mg, 4.0 mmol) and methylamine (2M in THF, 10 mL, 20 mmol)) gave the intermediate 1-methyl-3-(4-methyl-6-morpholino-5-nitropyrimidin-2-yl)phenyl)urea (381 mg, 42% yield, MS (ESI): m/z 373.4), which was converted to the title compound (by following the procedure as described as in Example 1 step 3 and 4) as off-white solid (Yield: 105 mg, 12%). MS (ESI) m/z 352.3

### Experimental for the Preparation of 4-Morpholino-2-Aryl-7-Aminomethyl-5H-pyrrolo[3,2-d]pyrimidine (Scheme 2)

### Example 7

### General Procedure

### Preparation of {3-[4-morpholin-4-yl-7-(pyrrolidin-1-ylmethyl)-5H-pyrrolo[3,2-d]pyrimidin-2-yl]phenyl}methanol

To a stirred solution of [3-(4-morpholin-4-yl-5H-pyrrolo[3,2-d]pyrimidin-2-yl)phenyl]methanol (Example 3) (19 mg, 0.06 mmol) in acetic acid (80% in water, 1 mL) was added formaldehyde (37% in water, 19 mg, 0.24 mmol), followed by addition of pyrrolidine (13 mg, 0.18 mmol). The resulting mixture was heated at 60° C. for 6 h, and cooled to room temperature. The reaction mixture was concentrated in vacuum, and the residue was subjected to HPLC separation to give the title compound as off-white solid (12 mg, 52% yield). MS (ESI) m/z 394.4

### Example 8

### Preparation of [3-(4-morpholin-4-yl-7-{[(2-piperidin-1-ylethyl)amino]methyl}-5H-pyrrolo[3,2-d]pyrimidin-2-yl)phenyl]methanol

[3-(4-morpholin-4-yl-7-{[(2-piperidin-1-ylethyl)amino]methyl}-5H-pyrrolo[3,2-d]pyrimidin-2-yl)phenyl]methanol was prepared by following the procedure as described as in Example 7. Starting from [3-(4-morpholin-4-yl-5H-pyrrolo[3,2-d]pyrimidin-2-yl)phenyl]methanol (Example 3) (19 mg, 0.06 mmol) and 1-(2-aminoethyl)-piperidine (23 mg, 0.18 mmol) the title compound was obtained as off-white solid (9 mg, 34% yield). MS (ESI) m/z 451.4

### Example 9

### Preparation of [3-(4-morpholin-4-yl-7-{[(pyridin-3-ylmethyl)amino]methyl}-5H-pyrrolo[3,2-d]pyrimidin-2-yl)phenyl]methanol

[3-(4-morpholin-4-yl-7-{[(pyridin-3-ylmethyl)amino]methyl}-5H-pyrrolo[3,2-d]pyrimidin-2-yl)phenyl]methanol was prepared by following the procedure as described as in Example 7. Staring from [3-(4-morpholin-4-yl-5H-pyrrolo[3,2-d]pyrimidin-2-yl)phenyl]methanol (from Example 3) (19 mg, 0.06 mmol) and 3-(amino-methyl)pyridine (19 mg, 0.18 mmol) the title compound was isolated as off-white solid (8 mg, 32% yield). MS (ESI) m/z 431.4

### Example 10

### Preparation of 3-{7-[(4-methylpiperazin-1-yl)methyl]-4-morpholin-4-yl-5H-pyrrolo[3,2-d]pyrimidin-2-yl}phenyl)methanol

3-{7-[(4-methylpiperazin-1-yl)methyl]-4-morpholin-4-yl-5H-pyrrolo[3,2-d]pyrimidin-2-yl}phenyl)methanol was prepared by following the procedure as described in the Example 7. Starting from [3-(4-morpholin-4-yl-5H-pyrrolo[3,2-d]pyrimidin-2-yl)phenyl]methanol (from Example 3) (19 mg, 0.06 mmol) and 1-methylpiperazine (18 mg, 0.18 mmol) the title compound was isolated as off-white solid (15.6 mg, 62% yield). MS (ESI) m/z 423.4

### Example 11

### Preparation of {3-[4-morpholin-4-yl-7-(piperazin-1-ylmethyl)-5H-pyrrolo[3,2-d]pyrimidin-2-yl]phenyl}methanol

{3-[4-morpholin-4-yl-7-(piperazin-1-ylmethyl)-5H-pyrrolo[3,2-d]pyrimidin-2-yl]phenyl}methanol was prepared by following the procedure as described in Example 7. Starting from by [3-(4-morpholin-4-yl-5H-pyrrolo[3,2-d]pyrimidin-2-yl)phenyl]methanol (19 mg, 0.06 mmol) and piperazine (15 mg, 0.18 mmol), the title compound was isolated as off-white solid (8 mg, 33% yield). MS (ESI) m/z 409.4

### Example 12

### Preparation of 3-{7-[(dimethylamino)methyl]-4-morpholin-4-yl-5H-pyrrolo[3,2-d]pyrimidin-2-yl}phenyl)methanol

3-{7-[(dimethylamino)methyl]-4-morpholin-4-yl-5H-pyrrolo[3,2-d]pyrimidin-2-yl}phenyl)methanol was prepared by following the procedure as described as in Example 7. Starting from [3-(4-morpholin-4-yl-5H-pyrrolo[3,2-d]pyrimidin-2-yl)phenyl]methanol (from Example 3) (19 mg, 0.06 mmol) and dimethylamine (8 mg, 0.18 mmol) the title compound was isolated as off-white solid (14 mg, 64% yield). MS (ESI) m/z 368.3

### Example 13

### Preparation of 3-{7-[(diethylamino)methyl]-4-morpholin-4-yl-5H-pyrrolo[3,2-d]pyrimidin-2-yl}phenyl)methanol

3-{7-[(diethylamino)methyl]-4-morpholin-4-yl-5H-pyrrolo[3,2-d]pyrimidin-2-yl}phenyl)methanol was prepared by following the procedure as described as in Example 7. Starting from [3-(4-morpholin-4-yl-5H-pyrrolo[3,2-d]pyrimidin-2-yl)phenyl]methanol (from Example 3) (19 mg, 0.06 mmol) and diethylamine (13 mg, 0.18 mmol) the title compound was isolated as off-white solid (4.5 mg, 19% yield). MS (ESI) m/z 396.4

### Example 14

### Preparation of 3-{4-morpholin-4-yl-7-[(4-pyrimidin-2-ylpiperazin-1-yl)methyl]-5H-pyrrolo[3,2-d]pyrimidin-2-yl}phenyl)methanol

3-{4-morpholin-4-yl-7-[(4-pyrimidin-2-ylpiperazin-1-yl)methyl]-5H-pyrrolo[3,2-d]pyrimidin-2-yl}phenyl)methanol was prepared by following the procedure as described in Example 7. Starting from [3-(4-morpholin-4-yl-5H-pyrrolo[3,2-d]pyrimidin-2-yl)phenyl]methanol (from Example 3) (19 mg, 0.06 mmol) and 1-(2-pyrimidyl)piperazine dihydrochloride (43 mg, 0.18 mmol) the title compound was isolated as off-white solid (6 mg, 21% yield). MS (ESI) m/z 487.4

### Example 15

### Preparation of 3-{7-[(4-benzylpiperazin-1-yl)methyl]-4-morpholin-4-yl-5H-pyrrolo[3,2-d]pyrimidin-2-yl}phenyl)methanol

3-{7-[(4-benzylpiperazin-1-yl)methyl]-4-morpholin-4-yl-5H-pyrrolo[3,2-d]pyrimidin-2-yl}phenyl)methanol was prepared by following the procedure as described in Example 7. Starting from [3-(4-morpholin-4-yl-5H-pyrrolo[3,2-d]pyrimidin-2-yl)phenyl]methanol (from Example 3) (137 mg, 0.44 mmol) and 1-phenylpiperazine (116 mg, 0.66 mmol) the title compound was isolated as off-white solid (145 mg, 66% yield). MS (ESI) m/z 499.2

### Example 16

### Preparation of 3-(4-morpholin-4-yl-7-{[(pyridin-3-ylmethyl)amino]methyl}-5H-pyrrolo[3,2-d]pyrimidin-2-yl)phenol

3-(4-morpholin-4-yl-7-{[(pyridin-3-ylmethyl)amino]methyl}-5H-pyrrolo[3,2-d]pyrimidin-2-yl)phenol was prepared by following the procedure as described in Example 7. Starting from 3-(4-morpholin-4-yl-5H-pyrrolo[3,2-d]pyrimidin-2-yl)phenol (from Example 2) (24 mg, 0.08 mmol) and 3-(aminomethyl)pyridine (26 mg, 0.24 mmol) the title compound was isolated as off-white solid (TFA salt, 15.5 mg, 37% yield). MS (ESI) m/z 417.4

### Example 17

### Preparation of 3-[4-morpholin-4-yl-7-(pyrrolidin-1-ylmethyl)-5H-pyrrolo[3,2-d]pyrimidin-2-yl]phenol

3-[4-morpholin-4-yl-7-(pyrrolidin-1-ylmethyl)-5H-pyrrolo[3,2-d]pyrimidin-2-yl]phenol was prepared by following the procedure as described in Example 7. Starting from 3-(4-morpholin-4-yl-5H-pyrrolo[3,2-d]pyrimidin-2-yl)phenol (from Example 2) (24 mg, 0.08 mmol) and pyrrolidine (17 mg, 0.24 mmol) the title compound was isolated as off-white solid (TFA salt, 15.4 mg, 39% yield). MS (ESI) m/z 380.4.

### Example 18

### Preparation of 3-{7-[(dimethylamino)methyl]-4-morpholin-4-yl-5H-pyrrolo[3,2-d]pyrimidin-2-yl}phenol

3-{7-[(dimethylamino)methyl]-4-morpholin-4-yl-5H-pyrrolo[3,2-d]pyrimidin-2-yl}phenol was prepared by following the procedure as described in Example 7. Starting from 3-(4-morpholin-4-yl-5H-pyrrolo[3,2-d]pyrimidin-2-yl)phenol (Example 2) (24 mg, 0.08 mmol) and dimethylamine (40% in water, 27 mg, 0.24 mmol) the title compound was isolated as off-white solid (TFA salt, 24.4 mg, 65% yield). MS (ESI) m/z 354.3.

### Example 19

### Preparation of 3-{7-[(diethylamino)methyl]-4-morpholin-4-yl-5H-pyrrolo[3,2-d]pyrimidin-2-yl}phenol

3-{7-[(diethylamino)methyl]-4-morpholin-4-yl-5H-pyrrolo[3,2-d]pyrimidin-2-yl}phenol was prepared by following the procedure as described in Example 7. Starting from 3-(4-morpholin-4-yl-5H-pyrrolo[3,2-d]pyrimidin-2-yl)phenol (Example 2) (24 mg, 0.08 mmol) and diethylamine (18 mg, 0.24 mmol) the title compound was isolated as off-white solid (TFA salt, 18 mg, 45% yield). MS (ESI) m/z 382.3.

### Example 20

### Preparation of 3-{4-morpholin-4-yl-7-[(4-pyrimidin-2-ylpiperazin-1-yl)methyl]-5H-pyrrolo[3,2-d]pyrimidin-2-yl}phenol

3-{4-morpholin-4-yl-7-[(4-pyrimidin-2-ylpiperazin-1-yl)methyl]-5H-pyrrolo[3,2-d]pyrimidin-2-yl}phenol was prepared by following the procedure as described in Example 7. Starting from 3-(4-morpholin-4-yl-5H-pyrrolo[3,2-d]pyrimidin-2-yl)phenol (Example 2) (24 mg, 0.08 mmol) with 1-(2-pyrimidyl)piperazine dihydrochloride (57 mg, 0.24 mmol) the title compound was isolated as off-white solid (TFA salt, 15 mg, 32% yield). MS (ESI) m/z 473.5.

### Experimental for the Preparation of 4-Morpholino-2-Aryl-7-Substituted piperidin-4-yl-5H-pyrrolo[3,2-d]pyrimidine (Scheme 3)

### Step 1 (General Procedure)

### Example 21

### Preparation of 2-[3-(benzyloxy)phenyl]-4-morpholin-4-yl-7-(1,2,3,6-tetrahydropyridin-4-yl)-5H-pyrrolo[3,2-d]pyrimidine

To a solution of 2-[3-(benzyloxy)phenyl]-4-morpholin-4-yl-5H-pyrrolo[3,2-d]pyrimidine (Example 1) (500 mg, 1.29 mmol) in methanol (5 mL) was added KOH (362 mg, 6.45 mmol, 5 eq) and 4-piperidone monohydrate hydrochloride (495 mg, 3.23 mmol, 2.5 eq). The resulting solution was heated at 66° C. overnight. The mixture was cooled to room temperature, and concentrated under reduced pressure. The residue was subjected to HPLC separation to give the title compound as yellow solid (300 mg, 50% yield). MS (ESI) m/z 468.4

### Step 2 (General Procedure)

### Example 22

### Preparation of 3-(4-morpholin-4-yl-7-piperidin-4-yl-5H-pyrrolo[3,2-d]pyrimidin-2-yl)phenol

To a solution of 2-[3-(benzyloxy)phenyl]-4-morpholin-4-yl-7-(1,2,3,6-tetrahydropyridin-4-yl)-5H-pyrrolo[3,2-d]pyrimidine (from Example 21) (250 mg, 0.53 mmol) in methanol (20 mL) was added 10% Pd/C (50 mg). The resulting mixture was taken to hydrogenation (H₂, 50 psi) at room temperature overnight. The reaction mixture was filtered through a pad of Celite™. The filtration was concentrated in vacuum, and the residue was purified by HPLC to give the title compound as off-white solid (200 mg, 99% yield). MS (ESI) m/z 380.4.

### Step 3 (General Procedure)

### Example 23

### Preparation of 3-{7-[1-(4-fluorobenzyl)piperidin-4-yl]-4-morpholin-4-yl-5H-pyrrolo[3,2-d]pyrimidin-2-yl}phenol

To a solution of 3-(4-morpholin-4-yl-7-piperidin-4-yl-5H-pyrrolo[3,2-d]pyrimidin-2-yl)phenol (from Example 22) (22 mg, 0.058 mmol) in methanol (1 mL) was added 4-fluorobenzaldehyde (22 mg, 0.177 mmol), followed by addition of ZnCl₂ (24 mg, 0.174 mmol) and NaCNBH₃ (11 mg, 0.174 mmol). The resulting mixture was stirred at room temperature overnight. The solvent was removed under reduced pressure, and the residue was subjected to HPLC separation to give the title compound as off-white solid (TFA salt, 17.2 mg, 49% yield). MS (ESI) m/z 488.8.

### Example 24

### Preparation of {3-[4-morpholin-4-yl-7-(1,2,3,6-tetrahydropyridin-4-yl)-5H-pyrrolo[3,2-d]pyrimidin-2-yl]phenyl}methanol

To a solution of [3-(4-morpholin-4-yl-5H-pyrrolo[3,2-d]pyrimidin-2-yl)phenyl]methanol (Example 3) (482 mg, 1.55 mmol) in methanol (5 mL) were added KOH (434 mg, 7.75 mmol) and 4-piperidine monohydrate hydrochloride (600 mg, 3.9 mmol). The resulting solution was heated at 66° C. overnight. The mixture was cooled to room temperature, and concentrated under reduced pressure. The residue was subjected to HPLC separation to give the title compound as yellow solid (296 mg, 49% yield). MS (ESI) m/z 392.4.

### Example 25

### Preparation of {3-[7-(1-benzyl-1,2,3,6-tetrahydropyridin-4-yl)-4-morpholin-4-yl-5H-pyrrolo[3,2-d]pyrimidin-2-yl]phenyl}methanol

The titled compound was prepared by following the procedure as described in Example 21. Starting from [3-(4-morpholin-4-yl-5H-pyrrolo[3,2-d]pyrimidin-2-yl)phenyl]methanol (Example 3) (74 mg, 0.24 mmol) and 1-benzyl-4-piperidone (112 mg, 0.59 mmol) the title compound was isolated as yellow solid (53 mg, 46% yield). MS (ESI) m/z 482.4.

### Example 26

### Preparation of 3-[7-(1-benzylpiperidin-4-yl)-4-morpholin-4-yl-5H-pyrrolo[3,2-d]pyrimidin-2-yl]phenol

The titled compound was prepared by following the procedure as described in Example 23. Starting from 3-(4-morpholin-4-yl-7-piperidin-4-yl-5H-pyrrolo[3,2-d]pyrimidin-2-yl)phenol (Example 22) (22 mg, 0.058 mmol) and benzaldehyde (18 mg, 0.174 mmol) to the title compound was isolated as off-white solid (TFA salt, 19.2 mg, 57% yield). MS (ESI) m/z 470.8.

### Example 27

### Preparation of 3-{7-[1-(2-furylmethyl)piperidin-4-yl]-4-morpholin-4-yl-5H-pyrrolo[3,2-d]pyrimidin-2-yl}phenol

The titled compound was prepared by following the procedure as described in Example 23. Starting from 3-(4-morpholin-4-yl-7-piperidin-4-yl-5H-pyrrolo[3,2-d]pyrimidin-2-yl)phenol (Example 22) (22 mg, 0.058 mmol) and 2-furaldehyde (17 mg, 0.174 mmol) the title compound was isolated as off-white solid (TFA salt, 12.2 mg, 37% yield). MS (ESI) m/z 460.8.

### Example 28

### Preparation of 3-{7-[1-(1H-imidazol-2-ylmethyl)piperidin-4-yl]-4-morpholin-4-yl-5H-pyrrolo[3,2-d]pyrimidin-2-yl}phenol

The titled compound was prepared by following the procedure as described in Example 23. Starting from 3-(4-morpholin-4-yl-7-piperidin-4-yl-5H-pyrrolo[3,2-d]pyrimidin-2-yl)phenol (Example 22) (22 mg, 0.058 mmol) and 2-imidazole-carboxaldehyde (17 mg, 0.174 mmol) the title compound was isolated as off-white solid (TFA salt, 21.3 mg, 64% yield); MS (ESI) m/z 460.8.

### Example 29

### Preparation of 3-[7-(1-isobutylpiperidin-4-yl)-4-morpholin-4-yl-5H-pyrrolo[3,2-d]pyrimidin-2-yl]phenol

The titled compound was prepared by following the procedure as described in Example 23. Starting from 3-(4-morpholin-4-yl-7-piperidin-4-yl-5H-pyrrolo[3,2-d]pyrimidin-2-yl)phenol (Example 22) (22 mg, 0.058 mmol) and isobutyraldehyde (13 mg, 0.174 mmol) the title compound was obtained as off-white solid (TFA salt, 18.6 mg, 58% yield); MS (ESI) m/z 436.8.

### Example 30

### Preparation of 3-[7-(1-methylpiperidin-4-yl)-4-morpholin-4-yl-5H-pyrrolo[3,2-d]pyrimidin-2-yl]phenol

The titled compound was prepared by following the procedure as described in Example 23. Starting from 3-(4-morpholin-4-yl-7-piperidin-4-yl-5H-pyrrolo[3,2-d]pyrimidin-2-yl)phenol (Example 22) (22 mg, 0.058 mmol) and formaldehyde (37% in water, 14 mg, 0.174 mmol) the title compound was isolated as off-white solid (TFA salt, 17.1 mg, 58% yield); MS (ESI) m/z 394.7.

### Example 31

### Preparation of 3-[7-(1-cyclohexylpiperidin-4-yl)-4-morpholin-4-yl-5H-pyrrolo[3,2-d]pyrimidin-2-yl]phenol

The titled compound was prepared by following the procedure as described in Example 23. Starting from 3-(4-morpholin-4-yl-7-piperidin-4-yl-5H-pyrrolo[3,2-d]pyrimidin-2-yl)phenol (Example 22) (22 mg, 0.058 mmol) and cyclohexanone (17 mg, 0.174 mmol) the title compound was isolated as off-white solid (TFA salt, 25.5 mg, 76% yield). MS (ESI) m/z 462.5.

### Example 32

### Preparation of 3-{7-[1-(2-fluorobenzyl)piperidin-4-yl]-4-morpholin-4-yl-5H-pyrrolo[3,2-d]pyrimidin-2-yl}phenol

The titled compound was prepared by following the procedure as described in Example 23. Starting from 3-(4-morpholin-4-yl-7-piperidin-4-yl-5H-pyrrolo[3,2-d]pyrimidin-2-yl)phenol (Example 22) (22 mg, 0.058 mmol) and 2-fluorobenaldehyde (22 mg, 0.174 mmol) the title compound was isolated as off-white solid (TFA salt, 26.7 mg, 77% yield); MS (ESI) m/z 488.5.

### Example 33

### Preparation of 3-{4-morpholin-4-yl-7-[1-(1H-pyrrol-2-ylmethyl)piperidin-4-yl]-5H-pyrrolo[3,2-d]pyrimidin-2-yl}phenol

The titled compound was prepared by following the procedure as described in Example 23. Starting from 3-(4-morpholin-4-yl-7-piperidin-4-yl-5H-pyrrolo[3,2-d]pyrimidin-2-yl)phenol (Example 22) (22 mg, 0.058 mmol) and pyrrole-2-carboxazaldehyde (17 mg, 0.174 mmol) the title compound was isolated as off-white solid (TFA salt, 10 mg, 30% yield); MS (ESI) m/z 459.8.

### Example 34

### Preparation of 3-{7-[1-(2-chloro-4-fluorobenzyl)piperidin-4-yl]-4-morpholin-4-yl-5H-pyrrolo[3,2-d]pyrimidin-2-yl}phenol

The titled compound was prepared by following the procedure as described in Example 23. Starting from 3-(4-morpholin-4-yl-7-piperidin-4-yl-5H-pyrrolo[3,2-d]pyrimidin-2-yl)phenol (Example 22) (22 mg, 0.058 mmol) and 2-chloro-4-fluorobenaldehyde (27 mg, 0.174 mmol) the title compound was isolated as off-white solid (TFA salt, 32.2 mg, 87% yield); MS (ESI) m/z 522.5.

### Example 35

### Preparation of 3-(7-{1-[(6-chloropyridin-3-yl)methyl]piperidin-4-yl}-4-morpholin-4-yl-5H-pyrrolo[3,2-d]pyrimidin-2-yl)phenol

The titled compound was prepared by following the procedure as described in Example 23. Starting from 3-(4-morpholin-4-yl-7piperidin-4-yl-5H-pyrrolo[3,2-d]pyrimidin-2-yl)phenol (Example 22) (22 mg, 0.058 mmol) and 6-chloropyridine-3-carboxaldehyde (25 mg, 0.174 mmol) the title compound was isolated as off-white solid (TFA salt, 25.3 mg, 70% yield); MS (ESI) m/z 505.8.

### Example 36

### Preparation of tert-Butyl (2-{4-[2-(3-hydroxyphenyl)-4-morpholin-4-yl-5H-pyrrolo[3,2-d]pyrimidin-7-yl]piperidin-1-yl}ethyl)carbamate

The titled compound was prepared by following the procedure as described in Example 23. Starting from 3-(4-morpholin-4-yl-7-piperidin-4-yl-5H-pyrrolo[3,2-d]pyrimidin-2-yl)phenol (Example 22) (22 mg, 0.058 mmol) and N-Boc-2-aminoacetaldehyde (28 mg, 0.174 mmol) to give the title compound was isolated as off-white solid (TFA salt, 28.6 mg, 77% yield); MS (ESI) m/z 523.5.

### Example 37

### Preparation of 3-(4-morpholin-4-yl-7-{1-[(4-morpholin-4-ylpyridin-3-yl)methyl]piperidin-4-yl}-5H-pyrrolo[3,2-d]pyrimidin-2-yl)phenol

The titled compound was prepared by following the procedure as described in Example 23. Starting from 3-(4-morpholin-4-yl-7-piperidin-4-yl-5H-pyrrolo[3,2-d]pyrimidin-2-yl)phenol (Example 22) (22 mg, 0.058 mmol) andl-morpholin-4-yl-pyridine-3-carboxaldehyde (33 mg, 0.174 mmol) the title compound was isolated as off-white solid (TFA salt, 25.4 mg, 65% yield); MS (ESI) m/z 556.6.

### Example 38

### Preparation of 3-{4-morpholin-4-yl-7-[1-(pyridin-2-ylmethyl)piperidin-4-yl]-5H-pyrrolo[3,2-d]pyrimidin-2-yl}phenol

The titled compound was prepared by following the procedure as described in Example 23. Starting from 3-(4-morpholin-4-yl-7-piperidin-4-yl-5H-pyrrolo[3,2-d]pyrimidin-2-yl)phenol (Example 22) (22 mg, 0.058 mmol) and 2-pyridine-carboxaldehyde (19 mg, 0.174 mmol) the title compound was isolated as off-white solid (TFA salt, 7.7 mg, 23% yield); MS (ESI) m/z 471.8.

### Example 39

### Preparation of 3-(7-{1-[(6-fluoropyridin-3-yl)methyl]piperidin-4-yl}-4-morpholin-4-yl-5H-pyrrolo[3,2-d]pyrimidin-2-yl)phenol

The titled compound was prepared by following the procedure as described in Example 23. Starting from 3-(4-morpholin-4-yl-7-piperidin-4-yl-5H-pyrrolo[3,2-d]pyrimidin-2-yl)phenol (Example 22) (22 mg, 0.058 mmol) and 2-fluoro-5-formylpyridine (22 mg, 0.174 mmol) the title compound was isolated as off-white solid (TFA salt, 10.8 mg, 31% yield); MS (ESI) m/z 489.5.

### Experimental for the Preparation of 4-Morpholino-2-(Urea)Aryl-5H-pyrrolo[3,2-d]pyrimidine (Scheme 4)

### Example 40

### General Procedure

### Preparation of 1-[2-(dimethylamino)ethyl]-3-[3-(4-morpholin-4-yl-5H-pyrrolo[3,2-d]pyrimidin-2-yl)phenyl]urea

To a solution of 3-(4-morpholin-4-yl-5H-pyrrolo[3,2-d]pyrimidin-2-yl)aniline (Example 5) (29 mg, 0.097 mmol) was added triethylamine (29 mg, 0.29 mmol) at 0° C., followed by addition of triphosgene (29 mg, 0.097 mmol) and N,N-dimethylethylenediamine (17 mg, 0.19 mmol). The resulting mixture was stirred at room temperature for 2 h, and concentrated under reduced pressure. The residue was subjected to HPLC separation to give the title compound as off-white solid (TFA salt, 44 mg, 84% yield); MS (ESI) m/z 410.2.

### Example 41

### Preparation of 1-(3-hydroxypropyl)-3-[3-(4-morpholin-4-yl-5H-pyrrolo[3,2-d]pyrimidin-2-yl)phenyl]urea

The titled compound was prepared by following the procedure as described in Example 40. Starting from 3-(4-morpholin-4-yl-5H-pyrrolo[3,2-d]pyrimidin-2-yl)aniline (Example 5) (29 mg, 0.097 mmol) and 3-amino-1-propanol (17 mg, 0.19 mmol) gave the title compound as off-white solid (TFA salt, 19.4 mg, 38% yield). MS (ESI) m/z 397.3.

### Example 42

### Preparation of 1-[3-(1H-imidazol-1-yl)propyl]-3-[3-(4-morpholin-4-yl-5H-pyrrolo[3,2-d]pyrimidin-2-yl)phenyl]urea

The titled compound was prepared by following the procedure as described in Example 40. Starting from 3-(4-morpholin-4-yl-5H-pyrrolo[3,2-d]pyrimidin-2-yl)aniline (Example 5) (29 mg, 0.097 mmol) and 1-(3-aminopropyl)-1-imidazole (24 mg, 0.19 mmol) to give the title compound as off-white solid (TFA salt, 20.4 mg, 36% yield). MS (ESI) m/z 447.4.

### Example 43

### Preparation of 1-(2-furylmethyl)-3-[3-(4-morpholin-4-yl-5H-pyrrolo[3,2-d]pyrimidin-2-yl)phenyl]urea

The titled compound was prepared by following the procedure as described in Example 40. Starting from 3-(4-morpholin-4-yl-5H-pyrrolo[3,2-d]pyrimidin-2-yl)aniline (Example 5) (29 mg, 0.097 mmol) and furfurylamine (18 mg, 0.19 mmol) to give the title compound as off-white solid (TFA salt, 20 mg, 38% yield). MS (ESI) m/z 419.3.

### Example 44

### Preparation of 1-[3-(4-morpholin-4-yl-5H-pyrrolo[3,2-d]pyrimidin-2-yl)phenyl]-3-(pyridin-3-ylmethyl)urea

The titled compound was prepared by following the procedure as described in Example 40. Starting from 3-(4-morpholin-4-yl-5H-pyrrolo[3,2-d]pyrimidin-2-yl)aniline (Example 5) (29 mg, 0.097 mmol) and 3-(aminomethyl)pyridine (21 mg, 0.19 mmol) the title compound was isolated as off-white solid (TFA salt, 13 mg, 24% yield). MS (ESI) m/z 430.3.

### Experimental for the Preparation of 4-morpholino-2-Aryl-5-substituted-5H-pyrrolo[3,2-d]pyrimidine (Scheme 5)

### Example 45

### General Procedure

### Preparation of [3-(5-methyl-4-morpholin-4-yl-5H-pyrrolo[3,2-d]pyrimidin-2-yl)phenyl]methanol

Step 1: Synthesis of 2-[3-({[tert-butyl(dimethyl)silyl]oxy}methyl)phenyl]-4-morpholin-4-yl-5H-pyrrolo[3,2-d]pyrimidine

To a solution of [3-(4-morpholin-4-yl-5H-pyrrolo[3,2-d]pyrimidin-2-yl)phenyl]methanol (Example 2) (1.469 g, 4.74 mmol) in DMF (5 mL) were added imidazole (0.483 g, 7.10 mmol) and tert-butyldimethylsilyl chloride (0.857 g, 5.69 mmol). The resulting mixture was heated at 80° C. for 15 min in microwave oven, and cooled to room temperature. The mixture was poured onto 20 mL of water, and extracted with EtOAc. The combined organic phases were washed with water and brine, and dried over MgSO₄. The solvent was removed under reduced pressure, and the residue was purified by flash chromatography (EtOAc:Hexanes=1:1) to give the title compound as white solid (1.949 g, 97% yield). MS (ESI) m/z 425.3.

Step 2: Synthesis of [3-(5-methyl-4-morpholin-4-yl-5H-pyrrolo[3,2-d]pyrimidin-2-yl)phenyl]methanol

To a solution of 2-[3-({[tert-butyl(dimethyl)silyl]oxy}methyl)phenyl]-4-morpholin-4-yl-5H-pyrrolo[3,2-d]pyrimidine (424 mg, 1.0 mmol) in THF (5 mL) was added NaH (60% in mineral oil, 80 mg, 2.0 mmol) at room temperature. After being stirred for 10 min, iodomethane (170 mg, 1.2 mmol) was added to the reaction mixture, and the resulting mixture was stirred at room temperature for 2 h. The reaction was quenched by addition of 2 mL of saturated aqueous ammonium chloride solution, followed by addition of 10 mL of water. The mixture was extracted with EtOAc, and combined organic phases were washed with water and brine, and dried over MgSO4. The solvent was removed under reduced pressure, and the residue was dissolved in 10 mL of CH₂Cl₂. To this solution was added drop wise of trifluoroacetic acid (TFA, 2 mL) at room temperature. The resulting mixture was stirred at room temperature for 3 h, and then diluted with CH₂Cl₂. The mixture was treated with 1N NaOH aqueous solution, and extracted with CH₂Cl₂. The organic extracts were washed with water and brine, and dried over MgSO₄. The solvent was removed under reduced pressure, and the residue was purified by flash chromatography to give the title compound as off-white solid (252 mg, 78 yield). MS (ESI) m/z 325.2.

### Example 46

### Preparation of Methyl {2-[3-(hydroxymethyl)phenyl]-4-morpholin-4-yl-5H-pyrrolo[3,2-d]pyrimidin-5-yl}acetate

Following the procedure as described in Example 45, reaction of 2-[3-({[tert-butyl(dimethyl)silyl]oxy}methyl)phenyl]-4-morpholin-4-yl-5H-pyrrolo[3,2-d]pyrimidine (Example 45, step 1) (424 mg, 1.0 mmol) and 2-iodoacetonitrile gave the intermediate 2-(2-(3-((tert-butyldimethylsilyoxy)methyl)phenyl)-4-morpholino-5H-pyrrolo[3,2-d]pyrimidin-5-yl)acetonitrile (353 mg, 76% yield, MS (ESI) m/z 464.3), which was treated with hydrochloric acid (4M in dioxane, 1 mL) in methanol (2 mL) to give the title compound as off-white solid (83 mg, 22% yield). MS (ESI) m/z 383.2.

### Example 47

### Preparation of {3-[5-methyl-4-morpholin-4-yl-7-(pyrrolidin-1-ylmethyl)-5H-pyrrolo[3,2-d]pyrimidin-2-yl]phenyl}methanol

To a solution of [3-(5-methyl-4-morpholin-4-yl-5H-pyrrolo[3,2-d]pyrimidin-2-yl)phenyl]methanol (Example 45) (100 mg, 0.31 mmol) in AcOH (80%, 1.5 mL) was added formaldehyde (37%, 100 mg, 1.24 mmol), followed by addition of pyrrolidine (65 mg, 0.93 mmol). The resulting mixture was heated at 60° C. overnight, and cooled to room temperature. The reaction mixture was concentrated in vacuum, and the residue was subjected to HPLC separation to give the title compound as off-white solid (32.8 mg, 26% yield); MS (ESI) m/z 408.3.

### Experimental for the Preparation of 4-(3-oxomorpholino)-2-Aryl-5H-pyrrolo[3,2-d]pyrimidine Compounds (Scheme 6)

### Example 48

### Preparation of 4-[2-(3-hydroxyphenyl)-5H-pyrrolo[3,2-d]pyrimidin-4-yl]morpholin-3-one

Step 1: Synthesis of tert-butyl [2-({2-[3-(benzyloxy)phenyl]-6-methyl-5-nitropyrimidin-4-yl}amino)ethoxy]acetate

To a solution of 2,4-dichloro-6-methyl-5-nitropyrimidine (716 mg, 3.46 mmol) in CH₂Cl₂ (10 mL) was added triethylamine (0.48 mL, 3.46 mmol) at 0° C., followed by addition of tert-butyl (2-amino-ethoxy)acetate (605 mg, 3.46 mmol). The resulting mixture was stirred at room temperature for 3 h, and diluted with 50 mL of CH₂Cl₂. The organic phase was washed with water and brine, and dried over MgSO₄. The solvent was removed under reduced pressure, and the residue was dissolved in 2 mL of DME. To this solution were added 3-benzyloxyphenylboronic acid (1.18 g, 5.19 mmol), Pd(PPh₃)₄ (200 mg, 5 mol %), and 1.5 mL of 2M Na₂CO₃ aqueous solution. The resulting mixture was heated at 110° C. for 30 min in microwave oven, and cooled to room temperature. The mixture was filtered, and washed with THF. The filtrate was concentrated under reduced pressure, and the residue was purified by flash chromatography to give the title compound as yellow solid (854 mg, 50% yield). MS (ESI) m/z 495.3

Step 2: Synthesis of [2-({2-[3-(benzyloxy)phenyl]-6-methyl-5-nitropyrimidin-4-yl}amino)ethoxy]acetic Acid

To a solution of tert-butyl [2-({2-[3-(benzyloxy)phenyl]-6-methyl-5-nitropyrimidin-4-yl}amino)ethoxy]acetate (233 mg, 0.47 mmol) in CH₂Cl₂ (5 mL) was added TFA (0.5 mL). The resulting mixture was stirred at room temperature overnight, and concentrated under reduced pressure. The residue was treated with hexanes, and the solid was collected by filtration to give the title compound as yellow solid (207 mg, 100% yield). MS (ESI) m/z 439.2.

Step 3: Synthesis of 4-{2-[3-(benzyloxy)phenyl]-6-methyl-5-nitropyrimidin-4-yl}morpholin-3-one

To a solution of [2-({2-[3-(benzyloxy)phenyl]-6-methyl-5-nitropyrimidin-4-yl}amino)ethoxy]acetic acid (207 mg, 0.47 mmol) in toluene (2 mL) and acetic anhydride (2 mL) was added pyridine (74 mg, 0.95 mmol) at room temperature. The resulting mixture was heated at 120° C. overnight, and concentrated under reduced pressure. The residue was purified by flash chromatography to give the title compound as light yellow solid (184 mg, 93% yield). MS (ESI) m/z 421.2.

Step 4: Synthesis of 4-{2-[3-(benzyloxy)phenyl]-6-[(E)-2-(dimethylamino)vinyl]-5-nitropyrimidin-4-yl}morpholin-3-one

A mixture of 4-{2-[3-(benzyloxy)phenyl]-6-methyl-5-nitropyrimidin-4-yl}morpholin-3-one (60 mg, 0.14 mmol) and dimethylformaldehyde N,N-dimethylformamide dimethyl acetal (DMFDMA) (1 mL) was heated at 170° C. for 15 min in microwave oven, and cooled to room temperature. The mixture was filtered through a short column (silica gel), washed with EtOAc. The filtrate was concentrated in vacuum to give the title compound as red solid (64 mg, 96% yield). MS (ESI) m/z 476.3.

Step 5: Synthesis of 4-[2-(3-hydroxyphenyl)-5H-pyrrolo[3,2-d]pyrimidin-4-yl]morpholin-3-one

A mixture of 4-{2-[3-(benzyloxy)phenyl]-6-[(E)-2-(dimethylamino)vinyl]-5-nitropyrimidin-4-yl}morpholin-3-one (64 mg, 0.13 mmol) and 10% Pd/C (20 mg) in methanol (5 mL) was taken to hydrogenation (H₂, 50 psi) at room temperature overnight. The mixture was filtered through a pad of Celite™, washed with methanol. The filtrate was concentrated in vacuum, and the residue was subjected to HPLC separation to give the title compound as off-white solid (5.8 mg, 14% yield). MS (ESI) m/z 311.3.

### Preparation of Examples 49-78

These compounds were made by essentially the methods described above, using suitable reaction conditions and starting materials. Those having ordinary skill in the art will understand how to select the appropriate conditions and materials to make each compound without undue experimentation.

The Compounds Shown in Table 1, Below, were Prepared According to the Above Procedures:

TABLE 1
1H-PYRROLO[3,2-D]PYRIMIDINE COMPOUNDS

| Example | Name | m/z |
|---|---|---|
| 1 | 2-[3-(benzyloxy)phenyl]-4-morpholin-4-yl- | 387.2 |
| | 5H-pyrrolo[3,2-d]pyrimidine | |
| 2 | 3-(4-morpholin-4-yl-5H-pyrrolo[3,2- | 297.1 |
| | d]pyrimidin-2-yl)phenol | |
| 3 | [3-(4-morpholin-4-yl-5H-pyrrolo[3,2- | 311.2 |
| | d]pyrimidin-2-yl)phenyl]methanol | |
| 4 | 2-(3-methylphenyl)-4-morpholin-4-yl-5H- | 295.2 |
| | pyrrolo[3,2-d]pyrimidine | |
| 5 | 3-(4-morpholin-4-yl-5H-pyrrolo[3,2- | 296.3 |
| | d]pyrimidin-2-yl)aniline | |
| 6 | 1-methyl-3-[4-(4-morpholin-4-yl-5H- | 352.3 |
| | pyrrolo[3,2-d]pyrimidin-2-yl)phenyl]urea | |
| 7 | {3-[4-morpholin-4-yl-7-(pyrrolidin-1- | 394.4 |
| | ylmethyl)-5H-pyrrolo[3,2-d]pyrimidin-2- | |
| | yl]phenyl}methanol | |
| 8 | [3-(4-morpholin-4-yl-7-{[(2-piperidin-1- | 451.4 |
| | ylethyl)amino]methyl}-5H-pyrrolo[3,2- | |
| | d]pyrimidin-2-yl)phenyl]methanol | |
| 9 | [3-(4-morpholin-4-yl-7-{[(pyridin-3- | 431.4 |
| | ylmethyl)amino]methyl}-5H-pyrrolo[3,2- | |
| | d]pyrimidin-2-yl)phenyl]methanol | |
| 10 | 3-{7-[(4-methylpiperazin-1-yl)methyl]-4- | 423.4 |
| | morpholin-4-yl-5H-pyrrolo[3,2-d]pyrimidin- | |
| | 2-yl}phenyl)methanol | |
| 11 | {3-[4-morpholin-4-yl-7-(piperazin-1- | 409.4 |
| | ylmethyl)-5H-pyrrolo[3,2-d]pyrimidin-2- | |
| | yl]phenyl}methanol | |
| 12 | 3-{7-[(dimethylamino)methyl]-4-morpholin- | 368.3 |
| | 4-yl-5H-pyrrolo[3,2-d]pyrimidin-2- | |
| | yl}phenyl)methanol | |
| 13 | 3-{7-[(diethylamino)methyl]-4-morpholin-4- | 396.4 |
| | yl-5H-pyrrolo[3,2-d]pyrimidin-2- | |
| | yl}phenyl)methanol | |
| 14 | 3-{4-morpholin-4-yl-7-[(4-pyrimidin-2- | 487.4 |
| | ylpiperazin-1-yl)methyl]-5H-pyrrolo[3,2- | |
| | d]pyrimidin-2-yl}phenyl)methanol | |
| 15 | 3-{7-[(4-benzylpiperazin-1-yl)methyl]-4- | 499.2 |
| | morpholin-4-yl-5H-pyrrolo[3,2-d]pyrimidin- | |
| | 2-yl}phenyl)methanol | |
| 16 | 3-(4-morpholin-4-yl-7-{[(pyridin-3- | 417.4 |
| | ylmethyl)amino]methyl}-5H-pyrrolo[3,2- | |
| | d]pyrimidin-2-yl)phenol | |
| 17 | 3-[4-morpholin-4-yl-7-(pyrrolidin-1- | 380.4 |
| | ylmethyl)-5H-pyrrolo[3,2-d]pyrimidin-2- | |
| | yl]phenol | |
| 18 | 3-{7-[(dimethylamino)methyl]-4-morpholin- | 354.3 |
| | 4-yl-5H-pyrrolo[3,2-d]pyrimidin-2-yl}phenol | |
| 19 | 3-{7-[(diethylamino)methyl]-4-morpholin-4- | 382.3 |
| | yl-5H-pyrrolo[3,2-d]pyrimidin-2-yl}phenol | |
| 20 | 3-{4-morpholin-4-yl-7-[(4-pyrimidin-2- | 473.5 |
| | ylpiperazin-1-yl)methyl]-5H-pyrrolo[3,2- | |
| | d]pyrimidin-2-yl}phenol | |
| 21 | 2-[3-(benzyloxy)phenyl]-4-morpholin-4-yl-7- | 468.4 |
| | (1,2,3,6-tetrahydropyridin-4-yl)-5H- | |
| | pyrrolo[3,2-d]pyrimidine | |
| 22 | 3-(4-morpholin-4-yl-7-piperidin-4-yl-5H- | 380.4 |
| | pyrrolo[3,2-d]pyrimidin-2-yl)phenol | |
| 23 | 3-{7-[1-(4-fluorobenzyl)piperidin-4-yl]-4- | 488.8 |
| | morpholin-4-yl-5H-pyrrolo[3,2-d]pyrimidin- | |
| | 2-yl}phenol | |
| 24 | {3-[4-morpholin-4-yl-7-(1,2,3,6- | 392.4 |
| | tetrahydropyridin-4-yl)-5H-pyrrolo[3,2- | |
| | d]pyrimidin-2-yl]phenyl}methanol | |
| 25 | {3-[7-(1-benzyl-1,2,3,6-tetrahydropyridin-4- | 482.4 |
| | yl)-4-morpholin-4-yl-5H-pyrrolo[3,2- | |
| | d]pyrimidin-2-yl]phenyl}methanol | |
| 26 | 3-[7-(1-benzylpiperidin-4-yl)-4-morpholin-4- | 470.8 |
| | yl-5H-pyrrolo[3,2-d]pyrimidin-2-yl]phenol | |
| 27 | 3-{7-[1-(2-furylmethyl)piperidin-4-yl]-4- | 460.8 |
| | morpholin-4-yl-5H-pyrrolo[3,2-d]pyrimidin- | |
| | 2-yl}phenol | |
| 28 | 3-{7-[1-(1H-imidazol-2-ylmethyl)piperidin-4- | 460.8 |
| | yl]-4-morpholin-4-yl-5H-pyrrolo[3,2- | |
| | d]pyrimidin-2-yl}phenol | |
| 29 | 3-[7-(1-isobutylpiperidin-4-yl)-4-morpholin-4- | 436.8 |
| | yl-5H-pyrrolo[3,2-d]pyrimidin-2-yl]phenol | |
| 30 | 3-[7-(1-methylpiperidin-4-yl)-4-morpholin-4- | 394.7 |
| | yl-5H-pyrrolo[3,2-d]pyrimidin-2-yl]phenol | |
| 31 | 3-[7-(1-cyclohexylpiperidin-4-yl)-4- | 462.5 |
| | morpholin-4-yl-5H-pyrrolo[3,2-d]pyrimidin- | |
| | 2-yl]phenol | |
| 32 | 3-{7-[1-(2-fluorobenzyl)piperidin-4-yl]-4- | 488.5 |
| | morpholin-4-yl-5H-pyrrolo[3,2-d]pyrimidin- | |
| | 2-yl}phenol | |
| 33 | 3-{4-morpholin-4-yl-7-[1-(1H-pyrrol-2- | 459.8 |
| | ylmethyl)piperidin-4-yl]-5H-pyrrolo[3,2- | |
| | d]pyrimidin-2-yl}phenol | |
| 34 | 3-{7-[1-(2-chloro-4-fluorobenzyl)piperidin-4- | 522.5 |
| | yl]-4-morpholin-4-yl-5H-pyrrolo[3,2- | |
| | d]pyrimidin-2-yl}phenol | |
| 35 | 3-(7-{1-[(6-chloropyridin-3- | 505.8 |
| | yl)methyl]piperidin-4-yl}-4-morpholin-4-yl- | |
| | 5H-pyrrolo[3,2-d]pyrimidin-2-yl)phenol | |
| 36 | tert-butyl (2-{4-[2-(3-hydroxyphenyl)-4- | 523.5 |
| | morpholin-4-yl-5H-pyrrolo[3,2-d]pyrimidin- | |
| | 7-yl]piperidin-1-yl}ethyl)carbamate | |
| 37 | 3-{7-[1-(2-chloro-4-fluorobenzyl)piperidin-4- | 522.5 |
| | yl]-4-morpholin-4-yl-5H-pyrrolo[3,2- | |
| | d]pyrimidin-2-yl}phenol | |
| 38 | 3-(7-{1-[(6-chloropyridin-3- | 505.8 |
| | yl)methyl]piperidin-4-yl}-4-morpholin-4-yl- | |
| | 5H-pyrrolo[3,2-d]pyrimidin-2-yl)phenol | |
| 39 | 3-(7-{1-[(6-fluoropyridin-3- | 489.5 |
| | yl)methyl]piperidin-4-yl}-4-morpholin-4-yl- | |
| | 5H-pyrrolo[3,2-d]pyrimidin-2-yl)phenol | |
| 40 | 1-[2-(dimethylamino)ethyl]-3-[3-(4- | 410.2 |
| | morpholin-4-yl-5H-pyrrolo[3,2-d]pyrimidin- | |
| | 2-yl)phenyl]urea | |
| 41 | 1-(3-hydroxypropyl)-3-[3-(4-morpholin-4-yl- | 397.3 |
| | 5H-pyrrolo[3,2-d]pyrimidin-2-yl)phenyl]urea | |
| 42 | 1-[3-(1H-imidazol-1-yl)propyl]-3-[3-(4- | 447.4 |
| | morpholin-4-yl-5H-pyrrolo[3,2-d]pyrimidin- | |
| | 2-yl)phenyl]urea | |
| 43 | 1-(2-furylmethyl)-3-[3-(4-morpholin-4-yl-5H- | 419.3 |
| | pyrrolo[3,2-d]pyrimidin-2-yl)phenyl]urea | |
| 44 | 1-[3-(4-morpholin-4-yl-5H-pyrrolo[3,2- | 430.3 |
| | d]pyrimidin-2-yl)phenyl]-3-(pyridin-3- | |
| | ylmethyl)urea | |
| 45 | [3-(5-methyl-4-morpholin-4-yl-5H- | 325.2 |
| | pyrrolo[3,2-d]pyrimidin-2-yl)phenyl]methanol | |
| 46 | methyl {2-[3-(hydroxymethyl)phenyl]-4- | 383.2 |
| | morpholin-4-yl-5H-pyrrolo[3,2-d]pyrimidin- | |
| | 5-yl}acetate | |
| 47 | {3-[5-methyl-4-morpholin-4-yl-7-(pyrrolidin- | 408.3 |
| | 1-ylmethyl)-5H-pyrrolo[3,2-d]pyrimidin-2- | |
| | yl]phenyl}methanol | |
| 48 | 4-[2-(3-hydroxyphenyl)-5H-pyrrolo[3,2- | 311.3 |
| | d]pyrimidin-4-yl]morpholin-3-one | |
| 49 | 1-[4-(4-morpholin-4-yl-5H-pyrrolo[3,2- | 415.2 |
| | d]pyrimidin-2-yl)phenyl]-3-pyridin-4-ylurea | |
| 50 | 1-[4-(5-benzyl-4-morpholin-4-yl-5H- | 505.2 |
| | pyrrolo[3,2-d]pyrimidin-2-yl)phenyl]-3- | |
| | pyridin-4-ylurea | |
| 51 | 1-[4-(5-methyl-4-morpholin-4-yl-5H- | 429.2 |
| | pyrrolo[3,2-d]pyrimidin-2-yl)phenyl]-3- | |
| | pyridin-4-ylurea (MW =) | |
| 52 | 1-[4-(4-morpholin-4-yl-5H-pyrrolo[3,2- | 540.3 |
| | d]pyrimidin-2-yl)phenyl]-3-{4-[(4- | |
| | methylpiperazin-1-yl)carbonyl]phenyl}urea | |
| 53 | 1-[4-(4-morpholin-4-yl-5H-pyrrolo[3,2- | 554.3 |
| | d]pyrimidin-2-yl)phenyl]-3-{4-[(4- | |
| | ethylpiperazin-1-yl)carbonyl]phenyl}urea | |
| 54 | 1-[4-(4-morpholin-4-yl-5H-pyrrolo[3,2- | 568.3 |
| | d]pyrimidin-2-yl)phenyl]-3-{4-[(4- | |
| | isopropylpiperazin-1-yl)carbonyl]phenyl}urea | |
| 55 | 1-[4-(4-morpholin-4-yl-5H-pyrrolo[3,2- | 526.3 |
| | d]pyrimidin-2-yl)phenyl]-3-{4-[(piperazin-1- | |
| | yl)carbonyl]phenyl}urea | |
| 56 | 1-[4-(4-morpholin-4-yl-5H-pyrrolo[3,2- | 568.3 |
| | d]pyrimidin-2-yl)phenyl]-3-{4-[(4- | |
| | (dimethylamino)piperidin-1- | |
| | yl)carbonyl]phenyl}urea | |
| 57 | N-[2-(dimethylamino)ethyl]-4-(1{[4-(4- | 542.3 |
| | morpholin-4-yl-5H-pyrrolo[3,2-d]pyrimidin- | |
| | 2-yl)phenyl]carbamoyl}amino)-N- | |
| | methylbenzamide | |
| 58 | N-[2-(dimethylamino)ethyl]-4-({[4-(4- | 528.3 |
| | morpholin-4-yl-5H-pyrrolo[3,2-d]pyrimidin- | |
| | 2-yl)phenyl]carbamoyl}amino)benzamide | |
| 59 | 4-({[4-(4-morpholin-4-yl-5H-pyrrolo[3,2- | 554.3 |
| | d]pyrimidin-2-yl)phenyl]carbamoyl}amino)- | |
| | N-(2-pyrrolidin-1-ylethyl)benzamide | |
| 60 | 1-[4-(4-morpholin-4-yl-5H-pyrrolo[3,2- | 594.3 |
| | d]pyrimidin-2-yl)phenyl]-3-{4-[(4-pyrrolidin- | |
| | 1-ylpiperidin-1-yl)carbonyl]phenyl}urea | |
| 61 | 1-[4-(5-methyl-4-morpholin-4-yl-5H- | 554.3 |
| | pyrrolo[3,2-d]pyrimidin-2-yl)phenyl]-3-{4- | |
| | [(4-methylpiperazin-1- | |
| | yl)carbonyl]phenyl}urea | |
| 62 | 1-[4-(5-methyl-4-morpholin-4-yl-5H- | 568.3 |
| | pyrrolo[3,2-d]pyrimidin-2-yl)phenyl]-3-{4- | |
| | [(4-ethylpiperazin-1-yl)carbonyl]phenyl}urea | |
| 63 | 1-[4-(5-methyl-4-morpholin-4-yl-5H- | 582.3 |
| | pyrrolo[3,2-d]pyrimidin-2-yl)phenyl]-3-{4- | |
| | [(4-isopropylpiperazin-1- | |
| | yl)carbonyl]phenyl}urea | |
| 64 | 1-[4-(5-methyl-4-morpholin-4-yl-5H- | 540.3 |
| | pyrrolo[3,2-d]pyrimidin-2-yl)phenyl]-3-{4- | |
| | [(piperazin-1-yl)carbonyl]phenyl}urea | |
| 65 | 1-[4-(5-methyl-4-morpholin-4-yl-5H- | 582.3 |
| | pyrrolo[3,2-d]pyrimidin-2-yl)phenyl]-3-{4- | |
| | [(4-(dimethylamino)piperidin-1- | |
| | yl)carbonyl]phenyl}urea | |
| 66 | N-[2-(dimethylamino)ethyl]-4-({[4-(5-methyl- | 556.3 |
| | 4-morpholin-4-yl-5H-pyrrolo[3,2-d]pyrimidin- | |
| | 2-yl)phenyl]carbamoyl}amino)-N- | |
| | methylbenzamide | |
| 67 | N-[2-(dimethylamino)ethyl]-4-({[4-(5-methyl- | 542.3 |
| | 4-morpholin-4-yl-5H-pyrrolo[3,2-d]pyrimidin- | |
| | 2-yl)phenyl]carbamoyl}amino)benzamide | |
| 68 | 4-({[4-(5-methyl-4-morpholin-4-yl-5H- | 568.3 |
| | pyrrolo[3,2-d]pyrimidin-2- | |
| | yl)phenyl]carbamoyl}amino)-N-(2-pyrrolidin- | |
| | 1-ylethyl)benzamide | |
| 69 | 1-[4-(5-methyl-4-morpholin-4-yl-5H- | 608.3 |
| | pyrrolo[3,2-d]pyrimidin-2-yl)phenyl]-3-{4- | |
| | [(4-pyrrolidin-1-ylpiperidin-1- | |
| | yl)carbonyl]phenyl}urea | |
| 70 | 1-[4-(5-ethyl-4-morpholin-4-yl-5H- | 568.3 |
| | pyrrolo[3,2-d]pyrimidin-2-yl)phenyl]-3-{4- | |
| | [(4-methylpiperazin-1- | |
| | yl)carbonyl]phenyl}urea | |
| 71 | 1-[4-(5-ethyl-4-morpholin-4-yl-5H- | 582.3 |
| | pyrrolo[3,2-d]pyrimidin-2-yl)phenyl]-3-{4- | |
| | [(4-ethylpiperazin-1-yl)carbonyl]phenyl}urea | |
| 72 | 1-[4-(5-ethyl-4-morpholin-4-yl-5H- | 596.3 |
| | pyrrolo[3,2-d]pyrimidin-2-yl)phenyl]-3-{4- | |
| | [(4-isopropylpiperazin-1- | |
| | yl)carbonyl]phenyl}urea | |
| 73 | 1-[4-(5-ethyl-4-morpholin-4-yl-5H- | 554.3 |
| | pyrrolo[3,2-d]pyrimidin-2-yl)phenyl]-3-{4- | |
| | [(piperazin-1-yl)carbonyl]phenyl}urea | |
| 74 | 1-[4-(5-ethyl-4-morpholin-4-yl-5H- | 596.3 |
| | pyrrolo[3,2-d]pyrimidin-2-yl)phenyl]-3-{4- | |
| | [(4-(dimethylamino)piperidin-1- | |
| | yl)carbonyl]phenyl}urea | |
| 75 | N-[2-(dimethylamino)ethyl]-4-({[4-(5-ethyl-4- | 570.3 |
| | morpholin-4-yl-5H-pyrrolo[3,2-d]pyrimidin-2- | |
| | yl)phenyl]carbamoyl}amino)-N- | |
| | methylbenzamide | |
| 76 | N-[2-(dimethylamino)ethyl]-4-({[4-(5-ethyl-4- | 556.3 |
| | morpholin-4-yl-5H-pyrrolo[3,2-d]pyrimidin-2- | |
| | yl)phenyl]carbamoyl}amino)benzamide | |
| 77 | 4-({[4-(5-ethyl-4-morpholin-4-yl-5H- | 582.3 |
| | pyrrolo[3,2-d]pyrimidin-2- | |
| | yl)phenyl]carbamoyl}amino)-N-(2-pyrrolidin- | |
| | 1-ylethyl)benzamide | |
| 78 | 1-[4-(5-ethyl-4-morpholin-4-yl-5H- | 622.3 |
| | pyrrolo[3,2-d]pyrimidin-2-yl)phenyl]-3-{4- | |
| | [(4-pyrrolidin-1-ylpiperidin-1- | |
| | yl)carbonyl]phenyl}urea | |

### Biological Evaluation MTOR Kinase Assay Methods

The routine human TOR assays with purified enzyme were performed in 96-well plates by DELFIA format as follows. Enzymes were first diluted in kinase assay buffer (10 mM HEPES (pH 7.4), 50 mM NaCl, 50 mM β-glycerophosphate, 10 mM MnCl₂, 0.5 mM DTT, 0.25 μM microcystin LR, and 100 μg/mL BSA). To each well, 12 μL of the diluted enzyme were mixed briefly with 0.5 μL test inhibitor or control vehicle dimethylsulfoxide (DMSO). The kinase reaction was initiated by adding 12.5 μL kinase assay buffer containing ATP and His6-S6K to give a final reaction volume of 25 μL containing 800 ng/mL FLAG-TOR, 100 μM ATP and 1.25 μM His6-S6K. The reaction plate was incubated for 2 hours (linear at 1-6 hours) at room temperature with gentle shaking and then terminated by adding 25 μL Stop buffer (20 mM HEPES (pH 7.4), 20 mM EDTA, 20 mM EGTA). The DELFIA detection of the phosphorylated (Thr-389) His 6-S6K was performed at room temperature using a monoclonal anti-P(T389)-p70S6K antibody (1A5, Cell Signaling) labeled with Europium-N1-ITC (Eu) (10.4 Eu per antibody, PerkinElmer). The DELFIA Assay buffer and Enhancement solution were purchased from PerkinElmer. 45 μL of the terminated kinase reaction mixture was transferred to a MaxiSorp plate (Nunc) containing 55 μL PBS. The His6-S6K was allowed to attach for 2 hours after which the wells were aspirated and washed once with PBS. 100 μL of DELFIA Assay buffer with 40 ng/mL Eu—P(T389)-S6K antibody was added. The antibody binding was continued for 1 hour with gentle agitation. The wells were then aspirated and washed 4 times with PBS containing 0.05% Tween-20 (PBST). 100 μL of DELFIA Enhancement solution was added to each well and the plates were read in a PerkinElmer Victor model plate reader. Data obtained were used to calculate enzymatic activity and enzyme inhibition by potential inhibitors.

### PI3K Fluorescence Polarization Assay Protocol Materials

Buffers: Reaction Buffer: 20 mM HEPES pH7.5, 2 mM MgCl₂, 0.05% CHAPS, and 0.01% bME (added fresh); STOP/detection Buffer: 100 mM HEPES pH7.5, 4 mM EDTA, 0.05% CHAPS. STOCKS: ATP 20 mM in H₂O; PIP2 (diC8, Echelon cat# P-4508) 1 mM in H₂O MW=856.5; GST-murine GRP 1.5 mg/ml in 17% glycerol; Red detector probe-Echelon (TAMRA) 2.5 uM. Plate: Nunc 384 well black polypropylene fluor. Plate.

### Methods

Assay: The assay is run by putting 9.5 ul of freshly diluted enzyme (in “reaction buffer”) per well, then 0.5 ul of diluted drug or DMSO mixing. Then 10 ul of substrate is added to start the reaction. The mixture is incubated 30-60 minutes, room temp, then stopped with 20 ul of stop/detector mix. The substrate solution, is 40 μM PIP2 and 50 uM ATP in reaction buffer. Addition of 10 ul of substrate to each well starts the reaction. This is 20 uM PIP2, 25 uM ATP final in reaction.

Stop/detector mix: 10 nM TAMRA detector, 40 nM GST-GRP in STOP/detection buffer. To stop reaction: add 20 ul Stop/detector mix per well and mix well. Wait 90-110 minutes before reading plate. Plates are read on Perkin-Elmer Envision plate readers with filters for Tamra. Keep Red probe solutions dark. This procedure is adapted from Echelon Biosciences Inc procedure for their PI3-Kinase fluorescence polarization activity Assay kit Product number K-1100.

### Cell Growth Assay

This assay measures the effect of compounds on cellular growth using human tumor derived cell lines of colon, melanoma, breast, ovarian, lung, and pancreatic origin. This assay system is conducted in SRB format. Cell exposure methodology in this procedure is also used to generate samples for analysis of lead compound suppression of phosphorylation various proteins that comprise Ras-MAPK and PI3 Kinase signaling pathways.

### In Vivo Anti-Tumor Efficacy Assays

Human tumor derived cell lines of colon, melanoma, breast, ovarian, lung, and pancreatic origin have been identified as capable of growing as xenografts in nude mice. Cell lines that display in vitro sensitivity to PI3K inhibitor compounds will be tested for ability to suppress tumor growth in vivo.

### Table 2 Shows the Results of the Described Biological Assays.

TABLE 2

| | TOR Kinase | PI3 Kinase α | PI3 Kinase γ |
|---|---|---|---|
| Example | IC₅₀ (μM) | IC₅₀ (nM) | IC₅₀ (nM) |
| 1 | >4.000 | 950 | 443 |
| 2 | 0.355 | 70 | 164 |
| 3 | 1.625 | 65 | 918 |
| 4 | 5 | 1506 | >8703 |
| 5 | 0.43 | 7500 | 8000 |
| 6 | 0.22 | 2666 | 4984 |
| 7 | 3.6 | 21 | 1122 |
| 8 | 11.15 | 72 | 4276 |
| 9 | 5.5 | 74 | 2874 |
| 10 | 7.85 | 62 | 2021 |
| 11 | 4.3 | 30 | 510 |
| 12 | 0.108 | 20 | 561 |
| 13 | 0.26 | 28 | 818 |
| 14 | 2.2 | 89 | 4506 |
| 15 | 5.8 | 240 | 2394 |
| 16 | >4.000 | 180 | 3030 |
| 17 | 3.25 | 105 | 1560 |
| 18 | 3.3 | 74 | 1152 |
| 19 | 3.1 | 132 | 1822 |
| 20 | 2 | 424 | 6702 |
| 21 | 7 | >10000 | >10000 |
| 22 | 5.9 | 354 | 1072 |
| 23 | 3.65 | 178 | 1066 |
| 24 | 3.4 | 410 | 1195 |
| 25 | 1.7 | 260 | 2788 |
| 26 | 2 | 340 | 890 |
| 27 | 2.75 | 297 | 1608 |
| 28 | 2.5 | 358 | 983 |
| 29 | 3.15 | 498 | 1897 |
| 30 | 3.75 | 484 | 828 |
| 31 | 0.37 | 250 | 2488 |
| 32 | 0.33 | 166 | 1923 |
| 33 | 0.96 | 111 | 792 |
| 34 | 0.46 | 120 | 1370 |
| 35 | 0.735 | 397 | 1450 |
| 36 | 0.58 | 222 | 1731 |
| 37 | 0.445 | 190 | 423 |
| 38 | 0.605 | 140 | 611 |
| 39 | 1.26 | 69 | 132 |
| 40 | >4.000 | 4684 | 9500 |
| 41 | >4.000 | 6220 | >10000 |
| 42 | >4.000 | 8938 | >10000 |
| 43 | >4.000 | >10000 | >10000 |
| 44 | >4.000 | >10000 | >10000 |
| 45 | >4.000 | 1996 | 8108 |
| 46 | >4.000 | 2399 | 2682 |
| 47 | >4.000 | 4207 | >10000 |
| 48 | 0.505 | 1183 | 1995 |

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compound of the Formula (I): 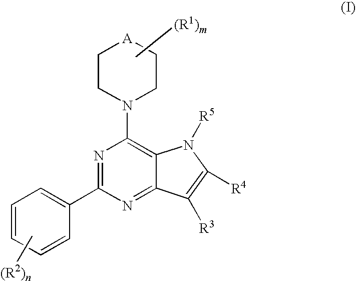
or a pharmaceutically acceptable salt thereof, wherein
R¹ is independently C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₂-C₆alkenyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₂-C₆alkynyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₃-C₈cycloalkyl optionally substituted with from 1 to 3 substituents independently selected from C₁-C₅alkyl, halo, halo(C₁-C₆alkyl)-, hydroxyl, —O—C₁-C₅alkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, carboxyamidoalkyl- and —NO₂; C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; or C₁-C₉heteroaryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂;
or two R¹ groups on the same carbon atom, when taken together with the carbon to which they are attached, form a carbonyl (C═O) group or two R¹ groups on the same carbon atom can be replaced by an alkylenedioxy group so that the alkylenedioxy group, when taken together with the carbon atom to which it is attached, form a 5- to 7-membered heterocycle containing two oxygen atoms;
A is —O—, —CH₂O—, —S—, —S(O)—, or S(O)₂—;
m is 0, 1, or 2;
R² is independently halogen; C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₁-C₆alkoxy optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₁-C₆alkoxycarbonyl; C₂-C₆alkenyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₂-C₆alkynyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₃-C₈cycloalkyl optionally substituted with from 1 to 3 substituents independently selected from C₁-C₅alkyl, halo, halo(C₁-C₆alkyl)-, hydroxyl, —O—C₁-C₅alkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, carboxyamidoalkyl- and —NO₂; C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; C₁-C₉heteroaryl optionally substituted with from 1 to 3 substituents independently selected from C₁-C₅alkyl, halo, halo(C₁-C₆alkyl)-, hydroxyl, C₁-C₅hydroxylalkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; hydroxyl; NR⁶R⁷; NO₂; CN; CO₂H; CF₃; CF₃O; C₁-C₆alkylthio; —SO₂NR⁶R⁷; —C(O)NR⁶R⁷; —NHC(O)NR⁶R⁷; —NHC(O)OR⁸; —NH(SO₂)NH—C₁-C₆alkyl; —NH(SO₂)NH—C₆-C₁₄aryl; —NHC(S)—NH—C₁-C₆alkyl; —N═C(S—C₁-C₆alkyl)(NH—C₁-C₆alkyl); —S(O)ₚ—C₆-C₁₄aryl; —S(O)ₚ—C₁-C₉heteroaryl; or —N(H)—C(═N—(CN))—(O—C₆-C₁₄aryl);
n is 1, 2, 3, 4, or 5;
each p is independently 1 or 2;
R⁶ and R⁷ are each independently H; C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, —NO₂, R¹¹R¹²NC(O)—, R¹¹R¹²NNHC(O)—, R¹¹O—, R¹¹R¹²N—, R¹¹R¹²NS(O)₂—, R¹¹S(O)₂NR¹²—, R¹¹R¹²NC(O)NH, R¹¹S—, R¹¹S(O)—, R¹¹S(O)₂—, and R¹¹C(O)—; C₁-C₉heteroaryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, —NO₂, R¹¹R¹²NC(O)—, R¹¹R¹²NNHC(O)—, R¹¹O—, R¹¹R¹²N—, R¹¹R¹²NS(O)₂—, R¹¹S(O)₂NR¹²—, R¹¹R¹²NC(O)NH, R¹¹S—, R¹¹S(O)—, R¹¹S(O)₂—, and R¹¹C(O)—; C₃-C₈cycloalkyl optionally substituted with from 1 to 3 substituents independently selected from C₁-C₅alkyl, halo, halo(C₁-C₆alkyl)-, hydroxyl, —O—C₁-C₅alkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, carboxyamidoalkyl- and —NO₂; or C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl;
or R⁶ and R⁷ when taken together with the nitrogen to which they are attached form a 3- to 7-membered nitrogen containing heterocycle wherein up to two of the carbon atoms of the heterocycle can be replaced with —N(R⁹)—, —O—, or —S(O)ₚ—;
R⁸ is C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; or C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂;
R⁹ is hydrogen; C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₃-C₈cycloalkyl optionally substituted with from 1 to 3 substituents independently selected from C₁-C₅alkyl, halo, halo(C₁-C₆alkyl)-, hydroxyl, —O—C₁-C₅alkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, carboxyamidoalkyl- and —NO₂; C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; C₁-C₉heteroaryl optionally substituted with from 1 to 3 substituents independently selected from C₁-C₅alkyl, halo, halo(C₁-C₆alkyl)-, hydroxyl, C₁-C₅hydroxylalkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; amino(C₁-C₆alkyl)-; or C₆-C₁₄arylamino;
R¹¹ and R¹² are each independently H, C₁-C₆alkoxy-, C₁-C₆alkyl-, C₁-C₆alkoxy(C₂-C₆alkylene)-, (C₁-C₆alkyl)amino-C₂-C₆alkylene-, di(C₁-C₆alkyl)amino-C₂-C₆alkylene-, C₂-C₆alkenyl, C₂-C₆alkynyl, C₆-C₁₄aryl-, (C₆-C₁₄aryl)alkyl-, C₃-C₈cycloalkyl-, C₁-C₉heteroaryl-, (C₁-C₉heteroaryl)alkyl-, C₁-C₉heterocyclyl- optionally substituted by C₁-C₆alkyl-, or heterocyclyl(C₁-C₆alkyl-);
or R¹¹ and R¹², when taken together with the nitrogen to which they are attached, form a 3- to 7-membered heterocycle wherein up to two of the carbon atoms of the heterocycle are optionally replaced with —N(H)—, —N(C₁-C₆alkyl)-, —N(C₃-C₈cycloalkyl)-, —N(C₆-C₁₄aryl)-, —N(C₁-C₉heteroaryl)-, —S—, —SO—, —S(O)₂—, or —O— and wherein any carbon atom of the heterocycle is optionally substituted with from 1 or 2 substituents independently selected from C₁-C₆alkyl-, H₂N—, (C₁-C₆alkyl)amino-, di(C₁-C₆alkyl)amino-, and C₁-C₉heterocyclyl-;
R³, R⁴, and R⁵ are independently H; C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₂-C₆alkenyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₂-C₆alkynyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; C₁-C₉heteroaryl optionally substituted with from 1 to 3 substituents independently selected from C₁-C₅alkyl, halo, halo(C₁-C₆alkyl)-, hydroxyl, C₁-C₅hydroxylalkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; NR⁶R⁷; C₁-C₆alkoxycarbonyl; perfluoroalkyl; —S(O)ₚ—C₆-C₁₄aryl; —S(O)ₚ—C₁-C₆alkyl; C(O)NR⁶R⁷; optionally substituted (C₆-C₁₄)arylalkyl- optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; heterocyclyl(C₁-C₆alkyl)- optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, (C₆-C₁₄aryl)alkyl-, —C(O)OH, —C(O)OC₁-C₆alkyl, —C(O)C₁-C₆alkyl, C₆-C₁₄ aryl, C₁-C₉heteroaryl, and C₃-C₈ cycloalkyl, wherein one of the CH₂ groups in the alkyl chain of the heterocyclyl(C₁-C₆alkyl)- can optionally be replaced by a NH group; 4- to 7-membered monocyclic heterocycle group optionally substituted with from 1 to 3 substituents independently selected from C₁-C₈acyl, C₁-C₆alkyl, heterocyclyl(C₁-C₆alkyl)-, wherein the ring portion of the heterocyclyl(C₁-C₆alkyl)- group is optionally substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, (C₆-C₁₄aryl)alkyl, wherein the ring portion of the (C₆-C₁₄aryl)alkyl group is optionally substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, (C₁-C₆-alkoxycarbonyl)-NH—(C₁-C₆)alkylene-, N-alkylamido-, —C(O)NH₂, (C₁-C₆alkyl)N-alkylamido-, and —NO₂; or C₃-C₈cycloalkyl optionally substituted with from 1 to 3 substituents independently selected from C₁-C₅alkyl, halo, halo(C₁-C₆alkyl)-, hydroxyl, —O—C₁-C₅alkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, carboxyamidoalkyl- and —NO₂.

2. A compound of claim 1 wherein R⁶ and R⁷ are each independently H; C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; C₁-C₉heteroaryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; C₃-C₈cycloalkyl optionally substituted with from 1 to 3 substituents independently selected from C₁-C₅alkyl, halo, halo(C₁-C₆alkyl)-, hydroxyl, —O—C₁-C₅alkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, carboxyamidoalkyl- and —NO₂; or C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

3. A compound of claim 1 of the Formula (VIII): 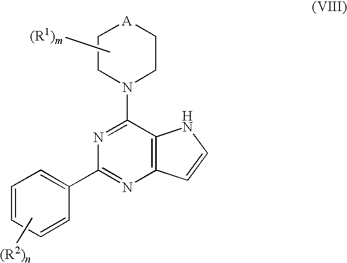
or a pharmaceutically acceptable salt thereof.

4. A compound of claim 1 of the Formula (XI): 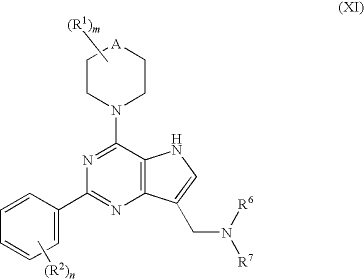
or a pharmaceutically acceptable salt thereof.

5. In another aspect, the invention provides compounds of the Formula (XVI): 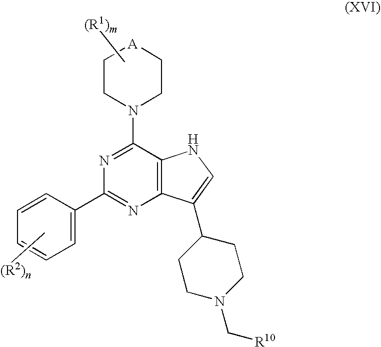
or a pharmaceutically acceptable salt thereof, wherein R¹⁰ is C₁-C₆alkyl or C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂.

6. A compound of claim 1 of the Formula (XIX): 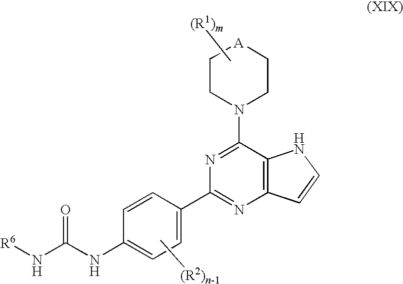
or a pharmaceutically acceptable salt thereof.

7. A compound of claim 1 of the Formula (XX): 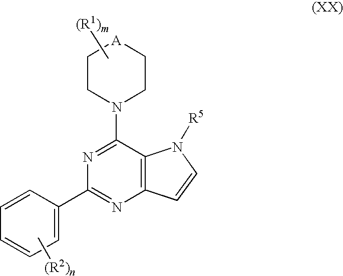
or a pharmaceutically acceptable salt thereof.

8. A compound of claim 1 of the Formula (XXIV): 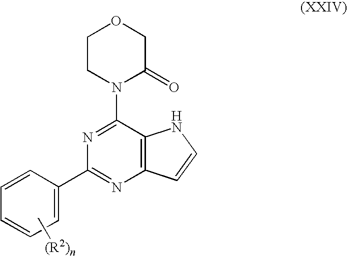
or a pharmaceutically acceptable salt thereof.

9. A compound of claim 1 wherein m is 0.

10. A compound of claim 1 wherein n is 1.

11. A compound of claim 1 wherein A is —O—.

12. A compound of claim 1 wherein R² is an optionally substituted urea of the formula —NHC(O)NR⁶R⁷, wherein R⁶ and R⁷ are each independently H; C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; C₁-C₉heteroaryl optionally substituted with from 1 to 3 substituents independently selected from C₁-C₅alkyl, halo, halo(C₁-C₆alkyl)-, hydroxyl, C₁-C₅hydroxylalkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; C₃-C₈cycloalkyl optionally substituted with from 1 to 3 substituents independently selected from C₁-C₅alkyl, halo, halo(C₁-C₆alkyl)-, hydroxyl, —O—C₁-C₅alkyl, —NH₂, -amino(C₁-C₆alkyl), (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, carboxyamidoalkyl- and —NO₂; or C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl;
or R⁶ and R⁷ when taken together with the nitrogen to which they are attached form a 3- to 7-membered nitrogen containing heterocycle wherein up to two of the carbon atoms of the heterocycle can be replaced with —N(R⁹)—, —O—, or —S(O)ₚ—;
R⁹ is hydrogen; C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl; C₃-C₈cycloalkyl optionally substituted with from 1 to 3 substituents independently selected from C₁-C₅alkyl, halo, halo(C₁-C₆alkyl)-, hydroxyl, —O—C₁-C₅alkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, carboxyamidoalkyl- and —NO₂; C₆-C₁₄aryl optionally substituted with from 1 to 3 substituents independently selected from C₁-C₅alkyl, halo, halo(C₁-C₆alkyl)-, hydroxyl, C₁-C₅hydroxylalkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; C₁-C₉heteroaryl optionally substituted with from 1 to 3 substituents independently selected from C₁-C₅alkyl, halo, halo(C₁-C₆alkyl)-, hydroxyl, C₁-C₅hydroxylalkyl, —NH₂, amino(C₁-C₆alkyl)-, (C₁-C₆alkyl)amino-, di(C₁-C₆alkyl)amino-, —COOH, —C(O)O—(C₁-C₅alkyl), —OC(O)—(C₁-C₅alkyl), (C₁-C₆alkyl)carboxyamido-, —C(O)NH₂, (C₁-C₆alkyl)N-alkylamido-, and —NO₂; amino(C₁-C₆alkyl)-; or C₆-C₁₄arylamino.

13. A compound of claim 1 wherein R² is C₁-C₆alkyl optionally substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

14. A compound of claim 13 wherein R² is the optionally substituted C₁-C₆alkyl group —CH₂OH.

15. A compound of claim 1 wherein R² is OH.

16. A compound of claim 15 wherein R² is OH in the meta position.

17. A compound of claim 1 wherein R² is amino.

18. A compound of claim 1 wherein R³ is H.

19. A compound of claim 1 wherein R³ is amino(C₁-C₆alkyl)optionally substituted with from 1 to 3 substituents independently selected from C₁-C₆alkoxy, C₆-C₁₄aryl, C₁-C₉heteroaryl, C₃-C₈cycloalkyl, and C₁-C₆alkyl.

20. A compound of claim 19 wherein R³ is di(C₁-C₆alkyl)aminomethyl.

21. A compound of claim 20 wherein R³ is dimethylaminomethyl.

22. A compound of claim 1 wherein R⁴ is H.

23. A compound of claim 1 wherein R⁵ is H.

24. A compound selected from the group consisting of:
2-[3-(benzyloxy)phenyl]-4-morpholin-4-yl-5H-pyrrolo[3,2-d]pyrimidine;
3-(4-morpholin-4-yl-5H-pyrrolo[3,2-d]pyrimidin-2-yl)phenol;
3-(4-morpholin-4-yl-5H-pyrrolo[3,2-d]pyrimidin-2-yl)phenyl]methanol;
2-(3-methylphenyl)-4-morpholin-4-yl-5H-pyrrolo[3,2-d]pyrimidine;
3-(4-morpholin-4-yl-5H-pyrrolo[3,2-d]pyrimidin-2-yl)aniline;
1-methyl-3-[4-(4-morpholin-4-yl-5H-pyrrolo[3,2-d]pyrimidin-2-yl)phenyl]urea;
{3-[4-morpholin-4-yl-7-(pyrrolidin-1-ylmethyl)-5H-pyrrolo[3,2-d]pyrimidin-2-yl]phenyl}methanol;
[3-(4-morpholin-4-yl-7-{[(2-piperidin-1-ylethyl)amino]methyl}-5H-pyrrolo[3,2-d]pyrimidin-2-yl)phenyl]methanol;
[3-(4-morpholin-4-yl-7-{[(pyridin-3-ylmethyl)amino]methyl}-5H-pyrrolo[3,2-d]pyrimidin-2-yl)phenyl]methanol;
3-{7-[(4-methylpiperazin-1-yl)methyl]-4-morpholin-4-yl-5H-pyrrolo[3,2-d]pyrimidin-2-yl}phenyl)methanol;
{3-[4-morpholin-4-yl-7-(piperazin-1-ylmethyl)-5H-pyrrolo[3,2-d]pyrimidin-2-yl]phenyl}methanol;
3-{7-[(dimethylamino)methyl]-4-morpholin-4-yl-5H-pyrrolo[3,2-d]pyrimidin-2-yl}phenyl)methanol;
3-{7-[(diethylamino)methyl]-4-morpholin-4-yl-5H-pyrrolo[3,2-d]pyrimidin-2-yl}phenyl)methanol;
3-{4-morpholin-4-yl-7-[(4-pyrimidin-2-ylpiperazin-1-yl)methyl]-5H-pyrrolo[3,2-d]pyrimidin-2-yl}phenyl)methanol;
3-{7-[(4-benzylpiperazin-1-yl)methyl]-4-morpholin-4-yl-5H-pyrrolo[3,2-d]pyrimidin-2-yl}phenyl)methanol;
3-(4-morpholin-4-yl-7-{[(pyridin-3-ylmethyl)amino]methyl}-5H-pyrrolo[3,2-d]pyrimidin-2-yl)phenol;
3-[4-morpholin-4-yl-7-(pyrrolidin-1-ylmethyl)-5H-pyrrolo[3,2-d]pyrimidin-2-yl]phenol;
3-{7-[(dimethylamino)methyl]-4-morpholin-4-yl-5H-pyrrolo[3,2-d]pyrimidin-2-yl}phenol;
3-{7-[(diethylamino)methyl]-4-morpholin-4-yl-5H-pyrrolo[3,2-d]pyrimidin-2-yl}phenol;
3-{4-morpholin-4-yl-7-[(4-pyrimidin-2-ylpiperazin-1-yl)methyl]-5H-pyrrolo[3,2-d]pyrimidin-2-yl}phenol;
2-[3-(benzyloxy)phenyl]-4-morpholin-4-yl-7-(1,2,3,6-tetrahydropyridin-4-yl)-5H-pyrrolo[3,2-d]pyrimidine;
3-(4-morpholin-4-yl-7-piperidin-4-yl-5H-pyrrolo[3,2-d]pyrimidin-2-yl)phenol;
3-{7-[1-(4-fluorobenzyl)piperidin-4-yl]-4-morpholin-4-yl-5H-pyrrolo[3,2-d]pyrimidin-2-yl}phenol;
{3-[4-morpholin-4-yl-7-(1,2,3,6-tetrahydropyridin-4-yl)-5H-pyrrolo[3,2-d]pyrimidin-2-yl]phenyl}methanol;
3-[7-(1-benzyl-1,2,3,6-tetrahydropyridin-4-yl)-4-morpholin-4-yl-5H-pyrrolo[3,2-d]pyrimidin-2-yl]phenyl}methanol;
3-[7-(1-benzylpiperidin-4-yl)-4-morpholin-4-yl-5H-pyrrolo[3,2-d]pyrimidin-2-yl]phenol;
3-{7-[1-(2-furylmethyl)piperidin-4-yl]-4-morpholin-4-yl-5H-pyrrolo[3,2-d]pyrimidin-2-yl}phenol;
3-{7-[1-(1H-imidazol-2-ylmethyl)piperidin-4-yl]-4-morpholin-4-yl-5H-pyrrolo[3,2-d]pyrimidin-2-yl}phenol;
3-[7-(1-isobutylpiperidin-4-yl)-4-morpholin-4-yl-5H-pyrrolo[3,2-d]pyrimidin-2-yl]phenol;
3-[7-(1-methylpiperidin-4-yl)-4-morpholin-4-yl-5H-pyrrolo[3,2-d]pyrimidin-2-yl]phenol;
3-[7-(1-cyclohexylpiperidin-4-yl)-4-morpholin-4-yl-5H-pyrrolo[3,2-d]pyrimidin-2-yl]phenol;
3-{7-[1-(2-fluorobenzyl)piperidin-4-yl]-4-morpholin-4-yl-5H-pyrrolo[3,2-d]pyrimidin-2-yl}phenol;
3-{4-morpholin-4-yl-7-[1-(1H-pyrrol-2-ylmethyl)piperidin-4-yl]-5H-pyrrolo[3,2-d]pyrimidin-2-yl}phenol;
3-{7-[1-(2-chloro-4-fluorobenzyl)piperidin-4-yl]-4-morpholin-4-yl-5H-pyrrolo[3,2-d]pyrimidin-2-yl}phenol;
3-(7-{1-[(6-chloropyridin-3-yl)methyl]piperidin-4-yl}-4-morpholin-4-yl-5H-pyrrolo[3,2-d]pyrimidin-2-yl)phenol;
tert-butyl (2-{4-[2-(3-hydroxyphenyl)-4-morpholin-4-yl-5H-pyrrolo[3,2-d]pyrimidin-7-yl]piperidin-1-yl}ethyl)carbamate;
3-(4-morpholin-4-yl-7-{1-[(4-morpholin-4-ylpyridin-3-yl)methyl]piperidin-4-yl}-5H-pyrrolo[3,2-d]pyrimidin-2-yl)phenol;
3-{4-morpholin-4-yl-7-[1-(pyridin-2-ylmethyl)piperidin-4-yl]-5H-pyrrolo[3,2-d]pyrimidin-2-yl}phenol;
3-(7-{1-[(6-fluoropyridin-3-yl)methyl]piperidin-4-yl}-4-morpholin-4-yl-5H-pyrrolo[3,2-d]pyrimidin-2-yl)phenol;
1-[2-(dimethylamino)ethyl]-3-[3-(4-morpholin-4-yl-5H-pyrrolo[3,2-d]pyrimidin-2-yl)phenyl]urea;
1-(3-hydroxypropyl)-3-[3-(4-morpholin-4-yl-5H-pyrrolo[3,2-d]pyrimidin-2-yl)phenyl]urea;
1-[3-(1H-imidazol-1-yl)propyl]-3-[3-(4-morpholin-4-yl-5H-pyrrolo[3,2-d]pyrimidin-2-yl)phenyl]urea;
1-(2-furylmethyl)-3-[3-(4-morpholin-4-yl-5H-pyrrolo[3,2-d]pyrimidin-2-yl)phenyl]urea;
1-[3-(4-morpholin-4-yl-5H-pyrrolo[3,2-d]pyrimidin-2-yl)phenyl]-3-(pyridin-3-ylmethyl)urea;
[3-(5-methyl-4-morpholin-4-yl-5H-pyrrolo[3,2-d]pyrimidin-2-yl)phenyl]methanol;
methyl {2-[3-(hydroxymethyl)phenyl]-4-morpholin-4-yl-5H-pyrrolo[3,2-d]pyrimidin-5-yl}acetate;
{3-[5-methyl-4-morpholin-4-yl-7-(pyrrolidin-1-ylmethyl)-5H-pyrrolo[3,2-d]pyrimidin-2-yl]phenyl}methanol;
4-[2-(3-hydroxyphenyl)-5H-pyrrolo[3,2-d]pyrimidin-4-yl]morpholin-3-one.

25. A compound selected from the group consisting of:
1-[4-(4-morpholin-4-yl-5H-pyrrolo[3,2-d]pyrimidin-2-yl)phenyl]-3-pyridin-4-ylurea;
1-[4-(5-benzyl-4-morpholin-4-yl-5H-pyrrolo[3,2-d]pyrimidin-2-yl)phenyl]-3-pyridin-4-ylurea;
1-[4-(5-methyl-4-morpholin-4-yl-5H-pyrrolo[3,2-d]pyrimidin-2-yl)phenyl]-3-pyridin-4-ylurea;
1-[4-(4-morpholin-4-yl-5H-pyrrolo[3,2-d]pyrimidin-2-yl)phenyl]-3-{4-[(4-methylpiperazin-1-yl)carbonyl]phenyl}urea;
1-[4-(4-morpholin-4-yl-5H-pyrrolo[3,2-d]pyrimidin-2-yl)phenyl]-3-{4-[(4-ethylpiperazin-1-yl)carbonyl]phenyl}urea;
1-[4-(4-morpholin-4-yl-5H-pyrrolo[3,2-d]pyrimidin-2-yl)phenyl]-3-{4-[(4-isopropylpiperazin-1-yl)carbonyl]phenyl}urea;
1-[4-(4-morpholin-4-yl-5H-pyrrolo[3,2-d]pyrimidin-2-yl)phenyl]-3-{4-[(piperazin-1-yl)carbonyl]phenyl}urea;
1-[4-(4-morpholin-4-yl-5H-pyrrolo[3,2-d]pyrimidin-2-yl)phenyl]-3-{4-[(4-(dimethylamino)piperidin-1-yl)carbonyl]phenyl}urea;
N-[2-(dimethylamino)ethyl]-4-({[4-(4-morpholin-4-yl-5H-pyrrolo[3,2-d]pyrimidin-2-yl)phenyl]carbamoyl}amino)-N-methylbenzamide;
N-[2-(dimethylamino)ethyl]-4-({[4-(4-morpholin-4-yl-5H-pyrrolo[3,2-d]pyrimidin-2-yl)phenyl]carbamoyl}amino)benzamide-4-({[4-(4-morpholin-4-yl-5H-pyrrolo[3,2-d]pyrimidin-2-yl)phenyl]carbamoyl}amino)-N-(2-pyrrolidin-1-ylethyl)benzamide;
1-[4-(4-morpholin-4-yl-5H-pyrrolo[3,2-d]pyrimidin-2-yl)phenyl]-3-{4-[(4-pyrrolidin-1-ylpiperidin-1-yl)carbonyl]phenyl}urea 1-[4-(5-methyl-4-morpholin-4-yl-5H-pyrrolo[3,2-d]pyrimidin-2-yl)phenyl]-3-{4-[(4-methylpiperazin-1-yl)carbonyl]phenyl}urea;
1-[4-(5-methyl-4-morpholin-4-yl-5H-pyrrolo[3,2-d]pyrimidin-2-yl)phenyl]-3-{4-[(4-ethylpiperazin-1-yl)carbonyl]phenyl}urea;
1-[4-(5-methyl-4-morpholin-4-yl-5H-pyrrolo[3,2-d]pyrimidin-2-yl)phenyl]-3-{4-[(4-isopropylpiperazin-1-yl)carbonyl]phenyl}urea;
1-[4-(5-methyl-4-morpholin-4-yl-5H-pyrrolo[3,2-d]pyrimidin-2-yl)phenyl]-3-{4-[(piperazin-1-yl)carbonyl]phenyl}urea;
1-[4-(5-methyl-4-morpholin-4-yl-5H-pyrrolo[3,2-d]pyrimidin-2-yl)phenyl]-3-{4-[(4-(dimethylamino)piperidin-1-yl)carbonyl]phenyl}urea;
N-[2-(dimethylamino)ethyl]-4-({[4-(5-methyl-4-morpholin-4-yl-5H-pyrrolo[3,2-d]pyrimidin-2-yl)phenyl]carbamoyl}amino)-N-methylbenzamide;
N-[2-(dimethylamino)ethyl]-4-({[4-(5-methyl-4-morpholin-4-yl-5H-pyrrolo[3,2-d]pyrimidin-2-yl)phenyl]carbamoyl}amino)benzamide-4-({[4-(5-methyl-4-morpholin-4-yl-5H-pyrrolo[3,2-d]pyrimidin-2-yl)phenyl]carbamoyl}amino)-N-(2-pyrrolidin-1-ylethyl)benzamide;
1-[4-(5-methyl-4-morpholin-4-yl-5H-pyrrolo[3,2-d]pyrimidin-2-yl)phenyl]-3-{4-[(4-pyrrolidin-1-ylpiperidin-1-yl)carbonyl]phenyl}urea;
1-[4-(5-ethyl-4-morpholin-4-yl-5H-pyrrolo[3,2-d]pyrimidin-2-yl)phenyl]-3-{4-[(4-methylpiperazin-1-yl)carbonyl]phenyl}urea;
1-[4-(5-ethyl-4-morpholin-4-yl-5H-pyrrolo[3,2-d]pyrimidin-2-yl)phenyl]-3-{4-[(4-ethylpiperazin-1-yl)carbonyl]phenyl}urea;
1-[4-(5-ethyl-4-morpholin-4-yl-5H-pyrrolo[3,2-d]pyrimidin-2-yl)phenyl]-3-{4-[(4-isopropylpiperazin-1-yl)carbonyl]phenyl}urea;
1-[4-(5-ethyl-4-morpholin-4-yl-5H-pyrrolo[3,2-d]pyrimidin-2-yl)phenyl]-3-{4-[(piperazin-1-yl)carbonyl]phenyl}urea;
1-[4-(5-ethyl-4-morpholin-4-yl-5H-pyrrolo[3,2-d]pyrimidin-2-yl)phenyl]-3-{4-[(4-(dimethylamino)piperidin-1-yl)carbonyl]phenyl}urea;
N-[2-(dimethylamino)ethyl]-4-({[4-(5-ethyl-4-morpholin-4-yl-5H-pyrrolo[3,2-d]pyrimidin-2-yl)phenyl]carbamoyl}amino)-N-methylbenzamide;
N-[2-(dimethylamino)ethyl]-4-({[4-(5-ethyl-4-morpholin-4-yl-5H-pyrrolo[3,2-d]pyrimidin-2-yl)phenyl]carbamoyl}amino)benzamide-4-({[4-(5-ethyl-4-morpholin-4-yl-5H-pyrrolo[3,2-d]pyrimidin-2-yl)phenyl]carbamoyl}amino)-N-(2-pyrrolidin-1-ylethyl)benzamide; and
1-[4-(5-ethyl-4-morpholin-4-yl-5H-pyrrolo[3,2-d]pyrimidin-2-yl)phenyl]-3-{4-[(4-pyrrolidin-1-ylpiperidin-1-yl)carbonyl]phenyl}urea.

26. A composition comprising a compound of claim 1 and a pharmaceutically acceptable carrier.

27. The composition of claim 26, wherein the pharmaceutically acceptable carrier is suitable for oral administration and the composition comprises an oral dosage form.

28. A composition comprising a compound of claim 1; a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, lavendustin A, hydroxyzine, glatiramer acetate, interferon beta-1a, interferon beta-1b, and natalizumab and lavendustin A; and a pharmaceutically acceptable carrier.

29. The composition of claim 28, wherein the second compound is Avastin.

30. A method of treating a PI3K-related disorder, comprising administering to a mammal in need thereof a compound of claim 1 in an amount effective to treat a PI3K-related disorder.

31. The method of claim 30, wherein the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

32. The method of claim 31, wherein the PI3K-related disorder is cancer.

33. The method of claim 32, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

34. A method of treating an mTOR-related disorder, comprising administering to a mammal in need thereof a compound of claim 1 in an amount effective to treat an mTOR-related disorder.

35. The method of claim 34, wherein the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

36. The method of claim 35, wherein the mTOR-related disorder is cancer.

37. The method of claim 36, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

38. A method of treating advanced renal cell carcinoma, comprising administering to a mammal in need thereof a compound of claim 1 in an amount effective to treat advanced renal cell carcinoma.

39. A method of treating acute lymphoblastic leukemia, comprising administering to a mammal in need thereof a compound of claim 1 in an amount effective to treat acute lymphoblastic leukemia.

40. A method of treating acute malignant melanoma, comprising administering to a mammal in need thereof a compound of claim 1 in an amount effective to treat malignant melanoma.

41. A method of treating soft-tissue or bone sarcoma, comprising administering to a mammal in need thereof a compound of claim 1 in an amount effective to treat soft-tissue or bone sarcoma.

42. A method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof the composition of claim 29 in an amount effective to treat the cancer.

43. A method of inhibiting mTOR in a subject, comprising administering to a subject in need thereof a compound of claim 1 in an amount effective to inhibit mTOR.

44. A method of inhibiting PI3K in a subject, comprising administering to a subject in need thereof a compound of claim 1 in an amount effective to inhibit PI3K.

45. A method of synthesizing compounds of the formula (VIII) comprising:
a) reacting 6-methyl-5-nitro-2,4-dichloropyrimidine of the Formula (II): 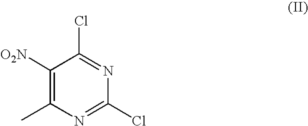
with a morpholine compound of the Formula (III); 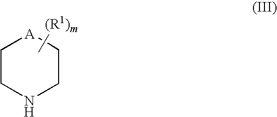
wherein R¹, A, and m are as defined in claim 1 to give the chloropyrimidine intermediate of Formula (IV): 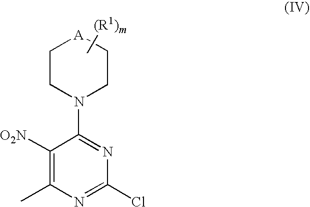
b) reacting the compound of Formula (IV) with a boronic acid of the structure (V): 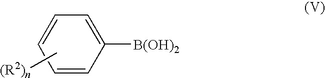
wherein R² and n are as defined in claim (I) thereby providing a compound of the Formula (VI): 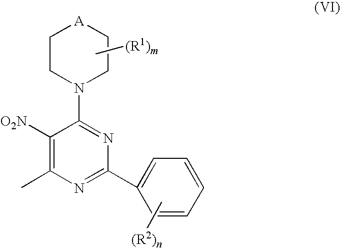
(c) reacting the compound of Formula (VI) with 1,1-dimethoxy-N,N-dimethylmethylamine (DMF-DMA) followed by reductive cyclization thereby providing a compound of the Formula (VIII): 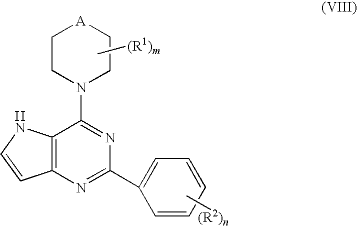
or a pharmaceutically acceptable salt thereof.

46. A method of synthesizing compounds of claim 1 comprising:
a) reacting 6-substituted-5-nitro-2,4-dihalopyrimidine of the Formula (XXV): 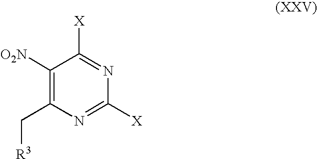
wherein X is a leaving group with a morpholine compound of the Formula (III); 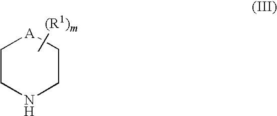
to give the halopyrimidine intermediate of Formula (XXVI): 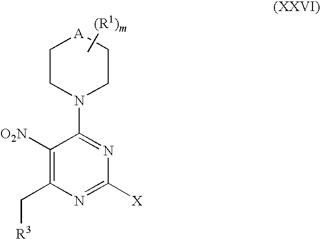
b) reacting the compound of Formula (XXVI) with a boronic acid of the structure (V): 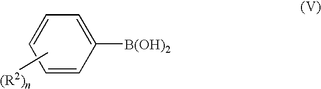
thereby providing a compound of the Formula (XXVII): 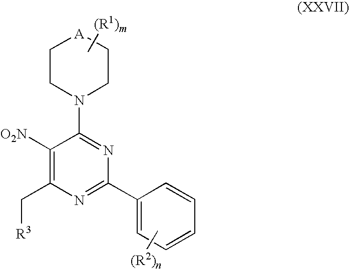
(c) reacting the compound of Formula (XXVII) with a 1,1-dimethoxy-N,N-dimethylalkylamine of the formula R⁴C(OCH₃)₂N(CH₃), followed by reductive cyclization thereby providing a compound of the Formula (XXIX): 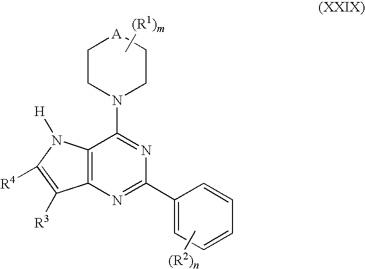
(d) reacting the compound of Formula (XXIX) at the pyrrole nitrogen by treating with sodium hydride and an alkylating agent R⁵X thereby providing a compound of the Formula (I): 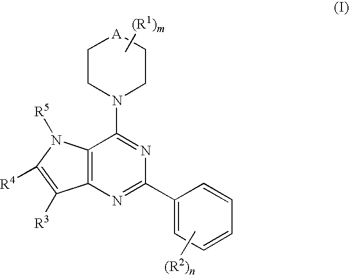
wherein X is a leaving group.
